(19) United States
(12) Patent Application Publication
(10) Pub. No.: US 2022/0389378 A1
(43) Pub. Date: Dec. 8, 2022

(54) SPIN-AGGREGATED NEURAL MICROSPHERES AND THE APPLICATION THEREOF

(71) Applicant: Novo Nordisk A/S, Bagsvaerd (DK)

(72) Inventors: Jonathan Niclis, Koebenhavn (DK); Josefine Raagaard Christiansen, Copenhagen N (DK); Henning Kempf, Vanloese (DK); Christian Le Fevre Honore, Vanloese (DK); Sofia Rebekka Boldt Schmidt, Koebenhavn N (DK)

(21) Appl. No.: 17/776,071

(22) PCT Filed: Nov. 20, 2020
(86) PCT No.: PCT/EP2020/082810

§ 371 Date: May 11, 2022

(30) Foreign Application Priority Data
Nov. 22, 2019 (EP) 19210844.7

(51) Int. Cl. C12N 5/0793

(52) U.S. Cl. CPC C12N 5/0619; C12N 2513/00; C12N 5/0012

(57) ABSTRACT

The present invention relates to a method for obtaining a neural micros-phere, comprising the steps of culturing pluripotent stem cells (PSCs), differentiating the PSCs into neural stem precursor blast cells, aggregating the neural stem precursor blast cells to form a neural microsphere, allowing the neural stem precursor blast cells of the neural microsphere to further mature, and collecting the neural microsphere.

### TECHNICAL FIELD

The present invention relates generally to the field of stem cells, such as human embryonic stem cells. Methods are provided for obtaining stem cell-derived neural cells. Specifically, methods are provided for obtaining stem cell-derived neural microspheres comprising the neural cells.

### BACKGROUND

The prospect of using human pluripotent stem cells in the treatment of various conditions seems very promising. Treatments include cell-replacement therapy of neurological conditions such as Parkinson's disease and stroke. For such treatments to become viable, however, it requires the development of in vitro methods to artificially produce the stem cell-derived products for their delivery to the central nervous system (CNS).

The process of differentiating human pluripotent stem cells (hPSCs) to neuronal lineages has become a highly efficient and robust process in 2D adherent culture formats. For this, hPSCs proceed through several stages that typically recapitulate development, by first differentiating from mitotic hPSCs to mitotic neural stem cells or neural precursor stem cells (NPSCs), then these differentiate (optionally) to a minimally mitotic or post-mitotic intermediate precursor, radial glial or neuroblast cell stage (collectively NB cells) and finally to post-mitotic neurons that are terminally differentiated. Differentiation from neural precursor stem or blast cells (NSPBCs) to neurons in 2D gives rise to cultures high in neuronal purity, and these cultures are characterized by extensive neurite outgrowth and interconnectivity between neurons. These cultures, however, are inherently, by their physical nature, architecturally/physically fragile networks. Some protocols exist that involve three-dimensional suspension culture steps that produce neurons in three-dimensional structures, but these do so at low efficiency by often comprising niches of mitotic neural cells and are typically very large and not suitable for transplantation to the CNS.

Extensive neurite growth, connectivity and fragility makes it impossible to physically manipulate, collect or concentrate two-dimensional adherent neuronal cultures for delivery to the CNS without incurring dramatic levels of cell death. To place additional neurons in a CNS, e.g. for cell therapy purposes, the primary method is to transplant preparations of neural stem precursor cells (NSPCs) and/or neuroblasts and allow for differentiation to proceed in vivo to neurons. NSPCs do not possess the fragile architecture of terminally differentiated neurons and can be disassociated with enzymes and/or chelators and concentrated as single (or 2-10 cell clusters) cell suspensions so that they may be loaded into thin diameter surgical delivery devices required for neurosurgery. Differentiation then proceeds in vivo to neurons and terminally differentiated cell types; yet, this process takes days up to months to complete and is not easily controlled as exogenous factors used to promote and guide this in vitro cannot be easily delivered to the CNS and are absent or minimally present in the adult brain.

The transplantation of NSPBCs to the CNS poses several problems. NSPBC preparations delivered to the CNS are comprised entirely or substantially of mitotic cells and these pose safety risks in terms of excessive growth or potential tumor formation. Furthermore, a primary method of administration for neural cell therapies is via the functional integration of neurons into host circuit networks. Mitotic neural stem cell preparations must undergo further division and differentiation in vivo to neurons before functional integration of transplanted cells is achieved, a process that occurs long after transplantation. NSPCs are also multipotent and have several terminal cell types that they may generate. NSPCs can mature to terminal cell types such as neurons either in vitro or in vivo. Control of the neural stem cell differentiation process is required to ensure highly homogenous terminally differentiated populations of desired subtypes. In vitro this process can be controlled in its entirety from start to finish, with treatment of cells with proteins and small molecules from the starting undifferentiated cells (hPSCs) all the way through to terminal/post-mitotic stage (i.e. neurons) and leads to cultures of high purity. Efficiencies of in vivo differentiation are dramatically lower by comparison. The difficulties in controlling in vivo differentiation is a consequence of the postnatal central nervous system being the transplant site for NSC therapies. Specifically, the brain is closed following transplantation and pro-differentiation molecules cannot be easily delivered to transplanted cells (and not without collateral effects on adjacent adult tissue). The adult brain does not produce developmental signals to guide differentiation, nor does it harbor developmental niches, guidance cues or the chemical gradients that control differentiation.

Neurospheres are a type of culture format that has been widely used since the emergence of neural differentiation protocols for human pluripotent stem cells (hPSCs) and are comprised principally of NSCs and progenitor cells. Neurospheres are importantly large in size, typically comprising many thousands of cells and measuring several hundred micrometers or even millimeters in diameter, and are always cultured in suspension and experience high levels of cell division and size increases over time due their formation from hPSCs themselves in most cases. These size dimensions in particular are not suitable for fitting within surgical devices and delivering to the brain of a patient. To differentiate neurospheres to neurons this presently requires either disassociation and grafting in vivo, or replating whole or disassociated spheres onto two-dimensional extracellular matrix-coated in vitro systems. Due to the large size of neurospheres, plating whole neurospheres results in poor attachment and thus technical challenges in maintaining these cells.

Presently all in vitro methodologies to mature NSCs to neurons (in high purity, >80% neurons) requires contact with extracellular matrices (ECMs). Dissociated monolayer 2D cultures of NSCs or whole re-plated or disassociated neurospheres require the addition of extracellular matrices (ECMs) for neurite formation and neuronal differentiation.

Alternative approaches for the generation of spheres comprising neural lineage cells, such as neurospheres, exists. However, these differ in several ways: Typically, they are generated by spontaneous aggregation, which does not produce spheres of consistent dimensions, produces spheres with heterogenous cell compositions, and results in spheres of larger dimensions. Spontaneously aggregated spheres and spin-aggregated sphere are typically reported to be of approx. 200-1000 μm in diameter and seeded with >1000 and up to 10,000 cells per sphere, sizes too large for delivery into the CNS using standard methodologies of delivery of a volume of cell solution via a cannula with diameters of approx. 70-120 μm (for rodents) or 900-1100 μm (for humans). These larger sizes typically possess necrotic cores in the center of spheres due to their heterogenous cell nature and problems with nutrient diffusion in such large sized cellular structures. Few instances of spin-aggregation methods have been reported for neural purposes and all use hPSCs as starting material. In using hPSCs as a starting material, these spin methods require patterning/differentiation and in vitro culture, that results in large neurospheres. Methods to address the above-mentioned challenges exist. WO2018096278 describes placing neurons in vivo without viability impairment, by performing the entire or part of a differentiation in 3D as small clusters of cells nested within layers of extracellular matrix proteins and solutions by custom encapsulation technologies and specialized bioreactors to culture and differentiate cells to neurons. However, the method according to WO2018096278 requires sophisticated custom bioprinting machinery and provides an end-product that, comprises an extracellular matrix and at least remnants of the encapsulation. This is undesirable from a pharmaceutical perspective when aiming to provide a patient-safe treatment.

It is therefore an object of the present invention to overcome the aforementioned challenges, in particular to provide a simple method for obtaining a pure stem cell-based neuronal product suitable for delivery to a patient.

### SUMMARY

The object as outlined above is achieved by the aspects of the present invention. In addition, the present invention may also solve further problems, which will be apparent from the disclosure of the exemplary embodiments.

A first aspect the present invention is to provide a method for obtaining a neural microsphere, comprising the steps of providing neural stem precursor blast cells, aggregating the neural stem precursor blast cells to form a neural microsphere, and allowing the neural stem precursor blast cells of the neural microsphere to further mature. In an embodiment, providing the neural stem precursor blast cells comprises the steps of culturing pluripotent stem cells (PSCs), and differentiating the PSCs into neural stem precursor blast cells. It follows that an aspect of the present invention relates to a neural microsphere comprising neural cells obtainable according to the aforementioned method. A further aspect of the present invention relates to the neural microsphere for the use as a pharmaceutical product. Specifically, it relates to the use of the neural microsphere for the treatment of a neural condition. In an embodiment of the present invention, the neural cells of the neural microsphere are midbrain neurons, such as dopaminergic progenitor cells, for the use in the treatment of Parkinson's disease.

The present invention provides a method for in vitro formation of neural microspheres in minimal (e.g. without the need for exogenous extracellular matrix components or other biomaterials such as hydrogel or alginate) and static non-adherent conditions via aggregation, such as controlled spin-aggregation, thereby enabling the generation of terminally differentiated progenies (particularly neurons) of controlled and minimal size suitable for direct loading into delivery devices for CNS transplantation. Accordingly, as opposed to traditional two-dimensional culturing methods the present invention does not require culturing in the presence of or being embedded within extracellular matrices and hence is a more pure and simplified method allowing cells to be matured to neural cells or other terminal cell types in minimal conditions. One advantage of the neural microsphere according to the present invention and compared to conventional 2D culture methods, is the smaller size. However, the method of aggregating the neural cells into a neural microsphere provides for conditions under which the neural cells mature in the neural microsphere substantially without an excessive outgrowth of neurites. The neural microsphere thereby comprises neural cells of a maturity which would normally only exist in an expanded, inseparable mesh having low survival rate and low ability to integrate when transplanted into the human brain. However, the mature neural cells of the neural microsphere maintain their properties and ability to form neurites once transferred to a different environment. This allows for very fast functional integration of neurons into host circuit networks, where the formation of neurites after engraftment into the brain will occur soon after. The present invention therefore overcomes the major challenge of providing neural cells, which have matured into their terminal fate for transplantation into the CNS. This significantly improves patient safety as the preparation delivered to the CNS is comprised entirely or substantially of post-mitotic cells, which poses little safety risks in terms of excessive growth or potential tumor formation. Furthermore, the method of the present invention allows for the control of the full differentiation process in vitro/by human operators, which ensures highly homogenous terminally differentiated populations of desired subtypes. It thereby overcomes the difficulties in controlling in vivo differentiation and maturation, which is a consequence of the postnatal central nervous system being the transplant site for NSC therapies. Finally, engraftment of matured neurons immediately provides the patient with the cells intended for therapy, thus eliminating the time it takes for further division and differentiation in vivo of the mitotic neural stem cells to neurons before functional integration of transplanted cells is achieved, a process which would otherwise occur long after transplantation.

The present invention further provides a more general aspect in a method for obtaining a stem cell-based microsphere, comprising the steps of differentiating PSCs to obtain differentiated cells, aggregating the differentiated cells to form a stem cell-based microsphere comprising cells, and allowing the cells of the stem cell-based microsphere to further mature. The present inventors found that this method is applicable to obtaining stem cell-based microspheres of any germ layer. In particular, the present inventors have demonstrated the method by obtaining microspheres comprising cardiomyocytes and pancreatic islet-like cells, respectively. The present inventors found that the smaller size and homogenous nature of the stem cell-based microspheres as obtained according to the methods of the invention result in a cell-based product with an increased viability when cryopreserved and subsequently thawed.

### BRIEF DESCRIPTION OF DRAWINGS

FIGS. 1-13 show characterization of hPSC-derived NSPBCs of the three main CNS lineages. FIG. 1 shows representative immunofluorescence images of dorsal forebrain NSPCs stained for DAPI (A, D), OTX2 (B), PAX6 (C) and SOX2 (E) after 19 days in vitro (DIV) of differentiation. Scale bar: 100 μm. FIG. 2 shows representative dot plots of dorsal forebrain

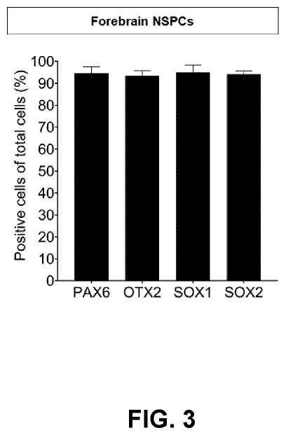

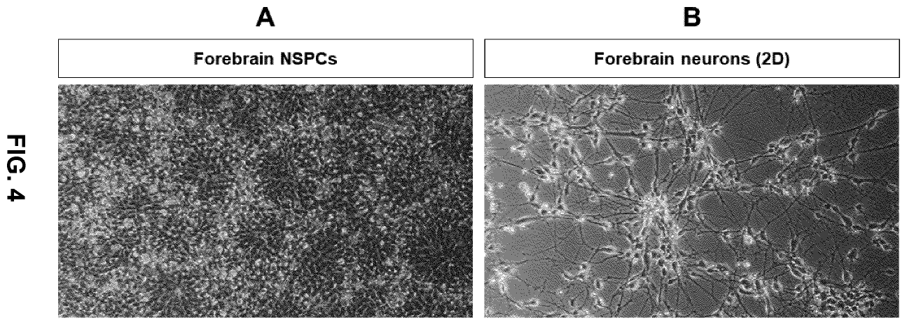

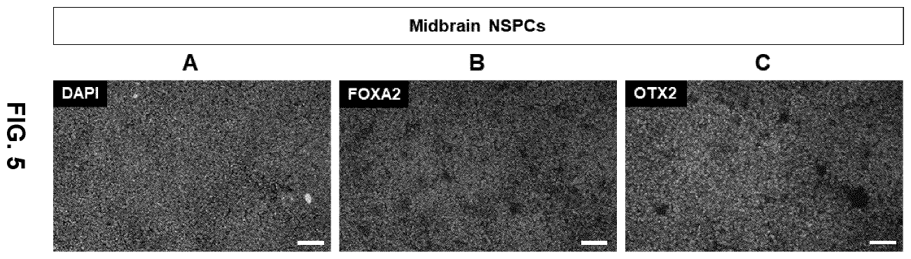

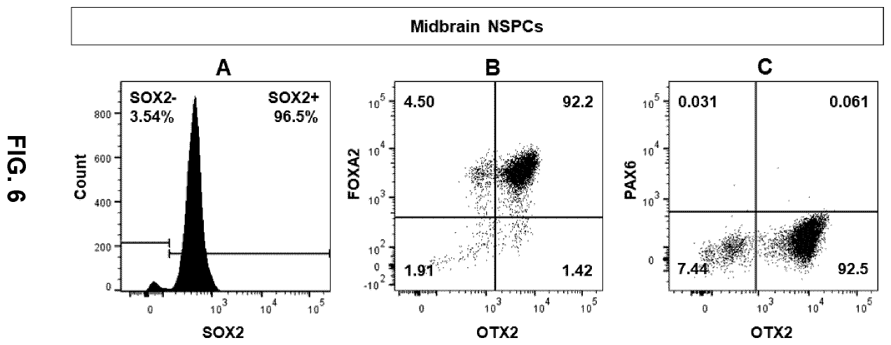

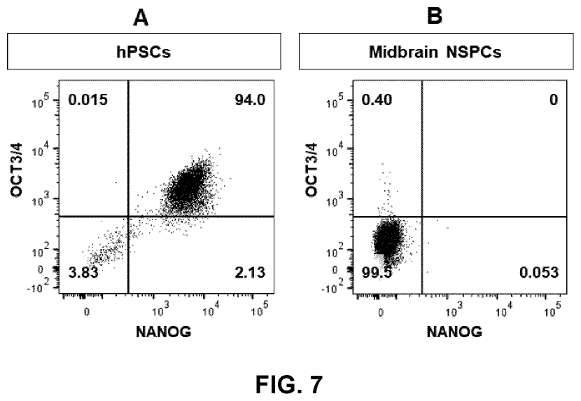

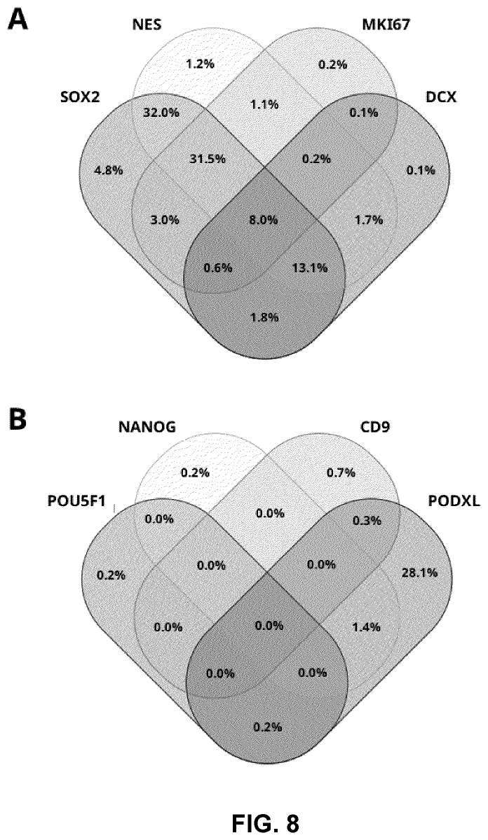

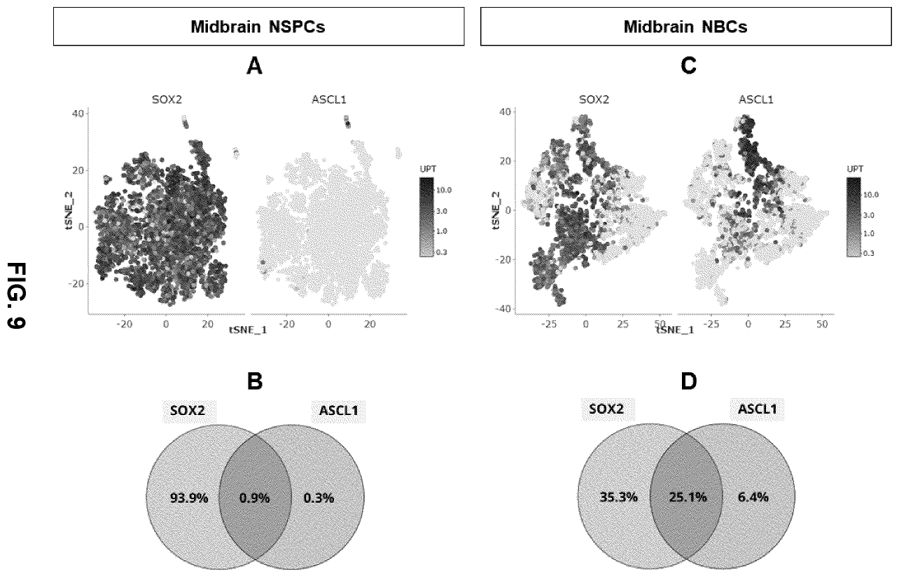

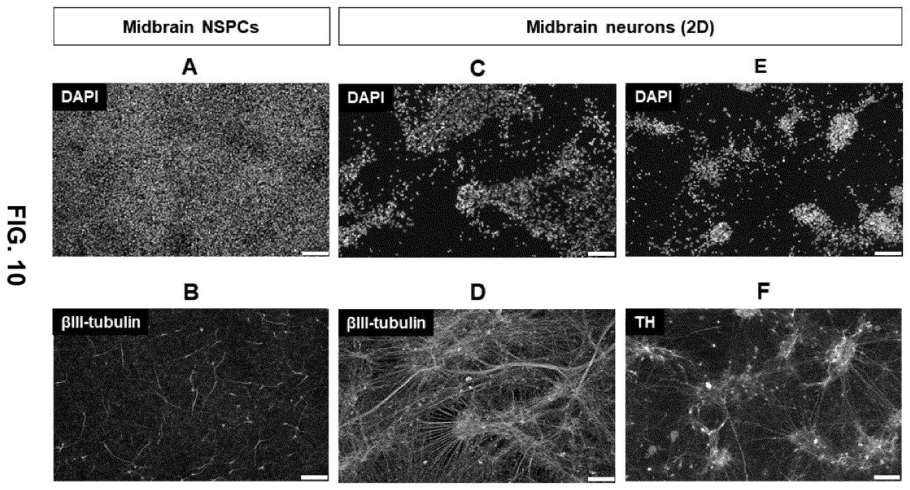

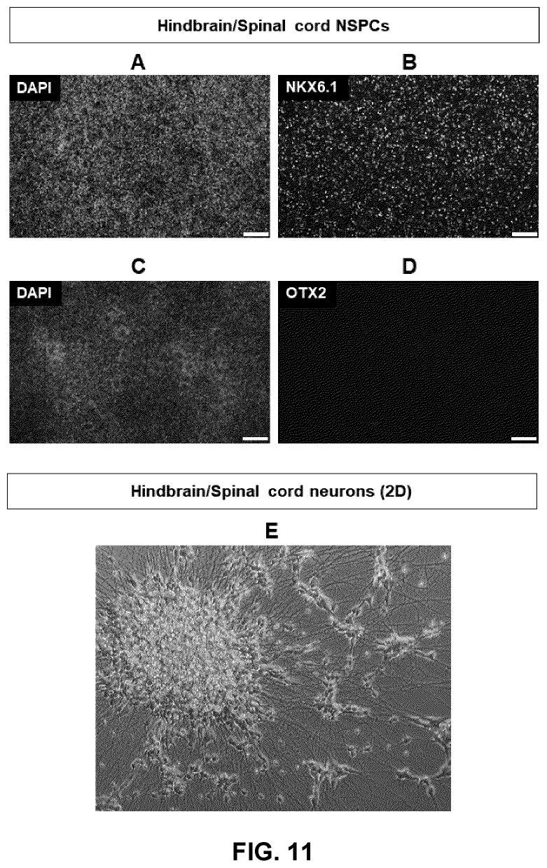

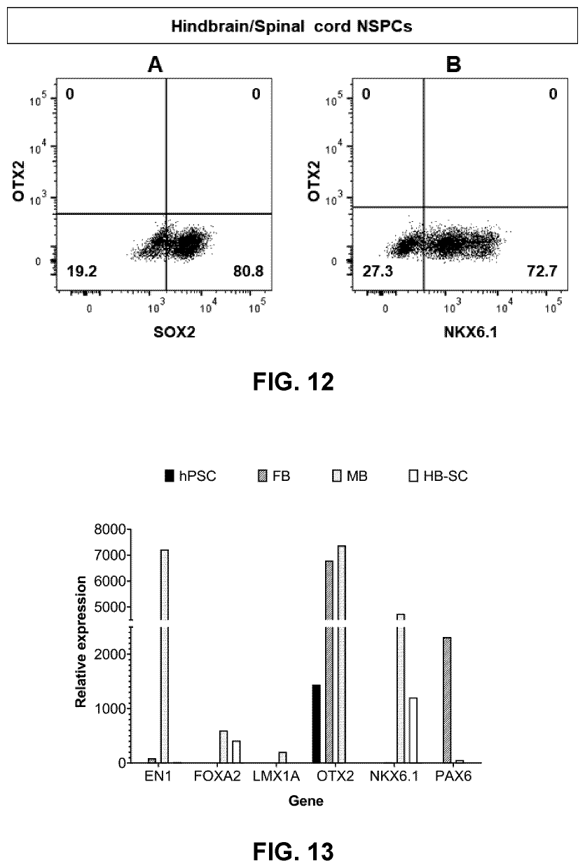

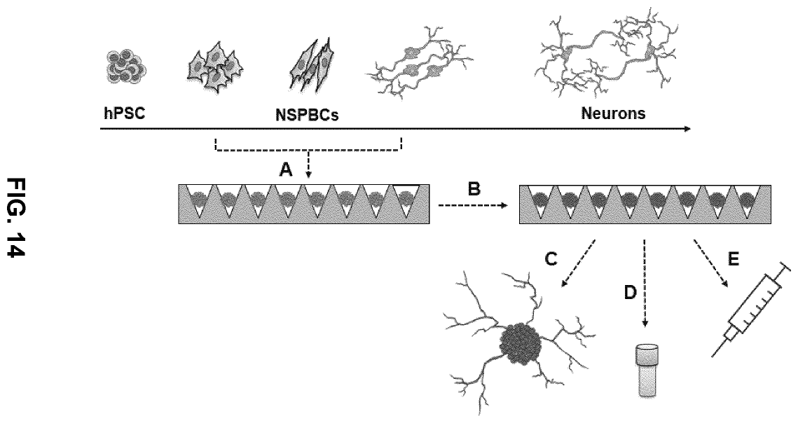

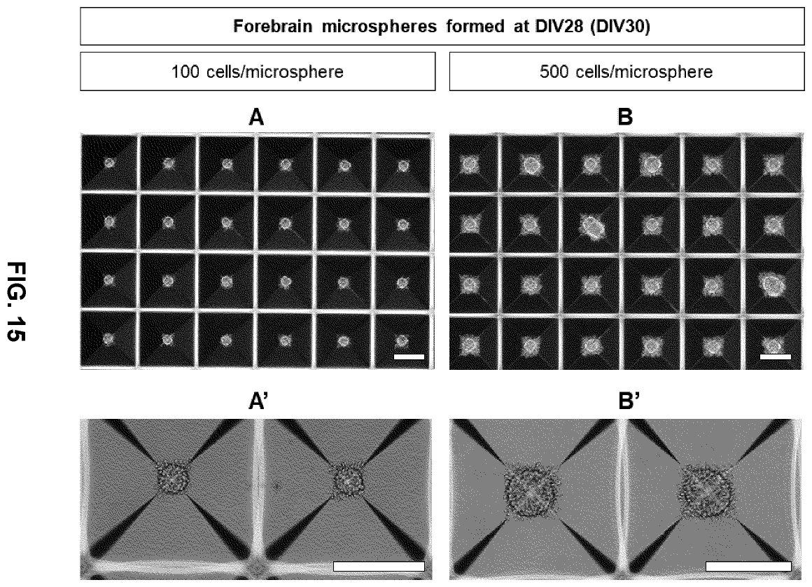

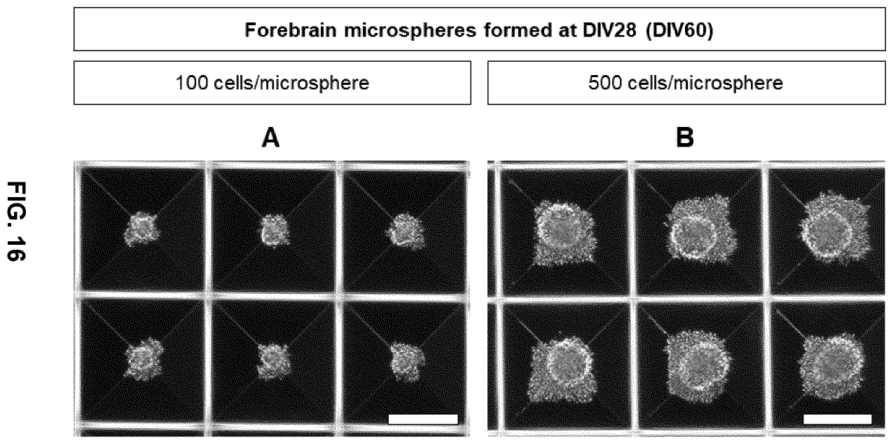

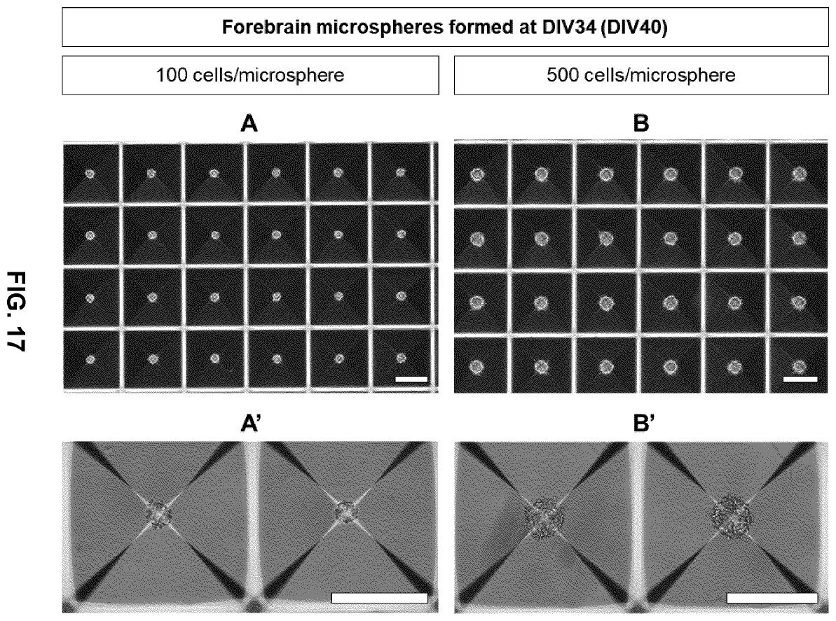

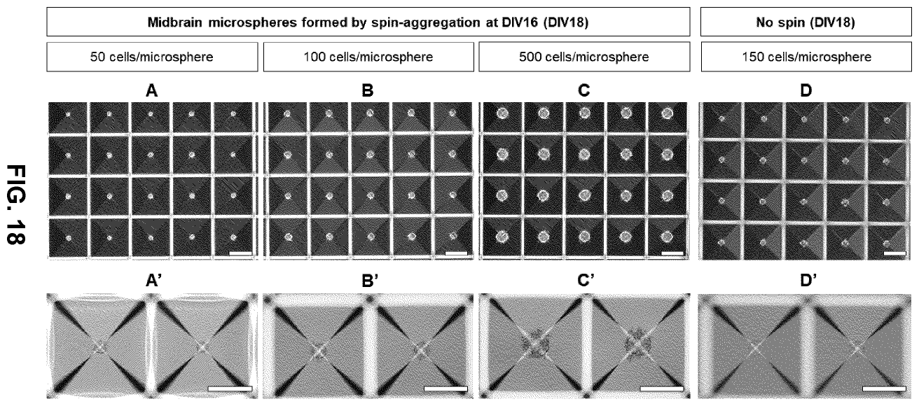

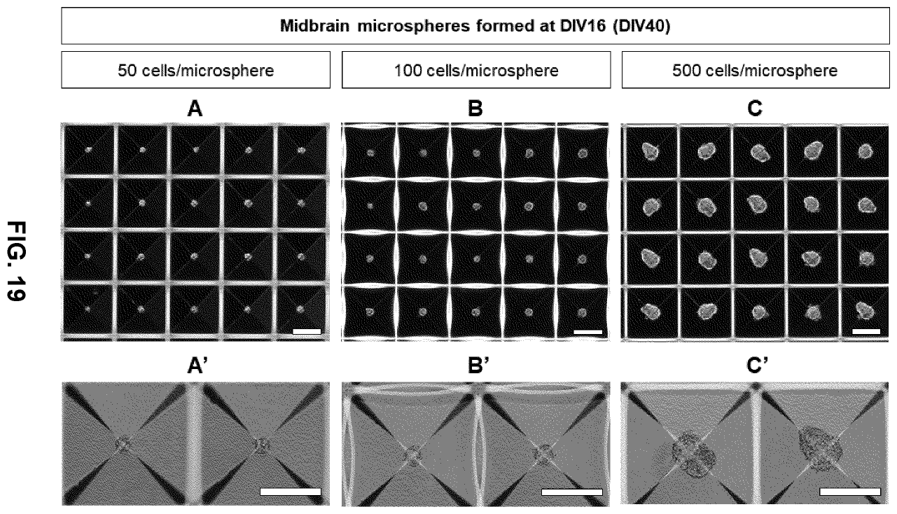

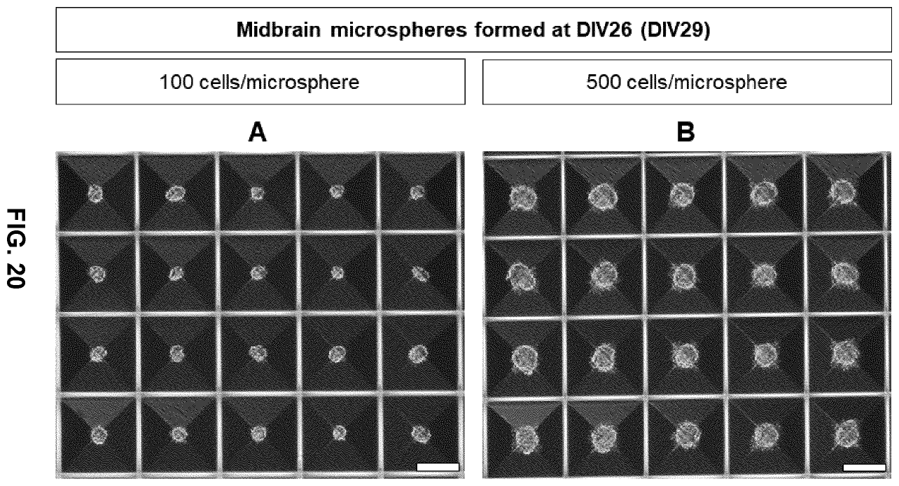

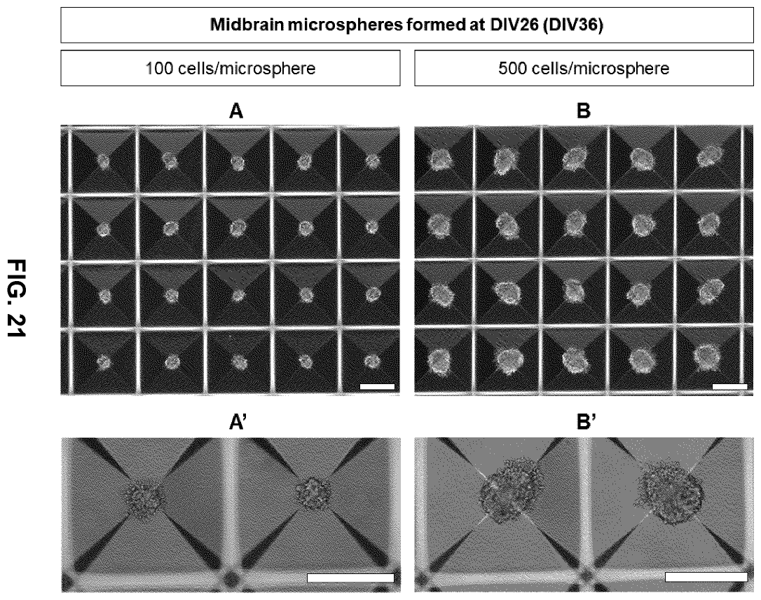

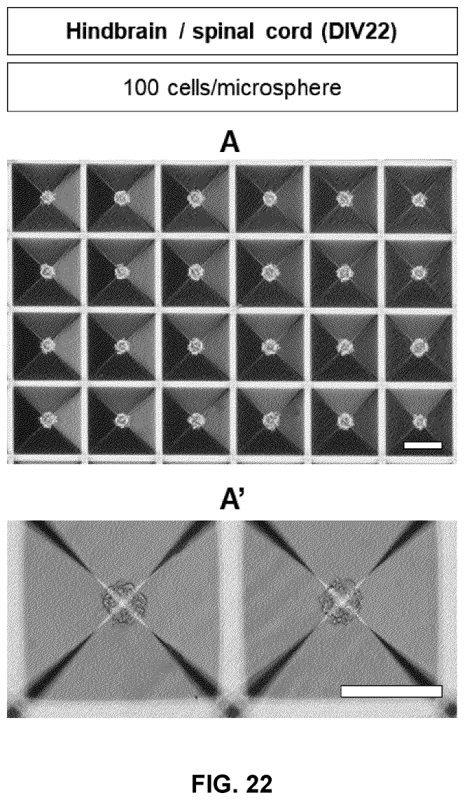

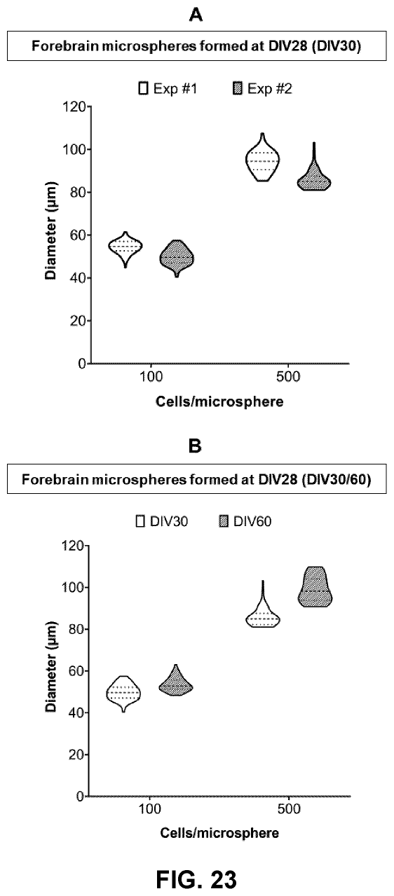

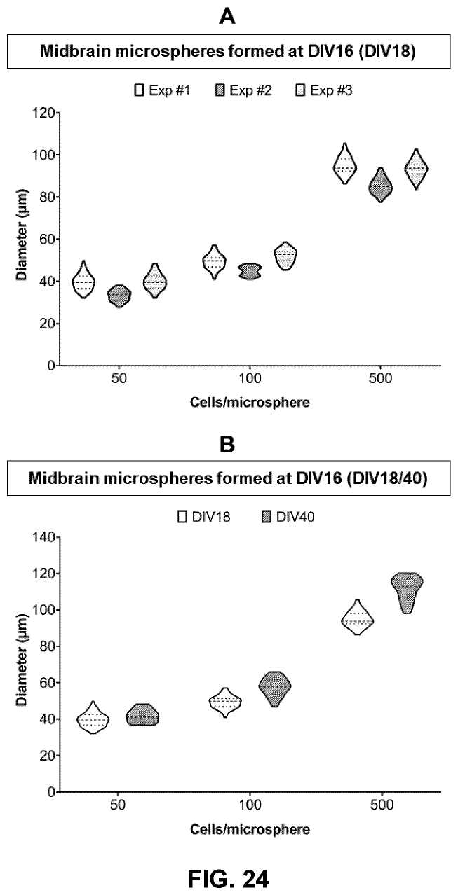

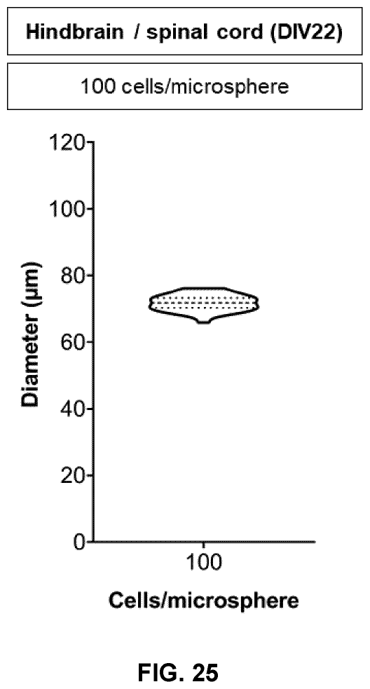

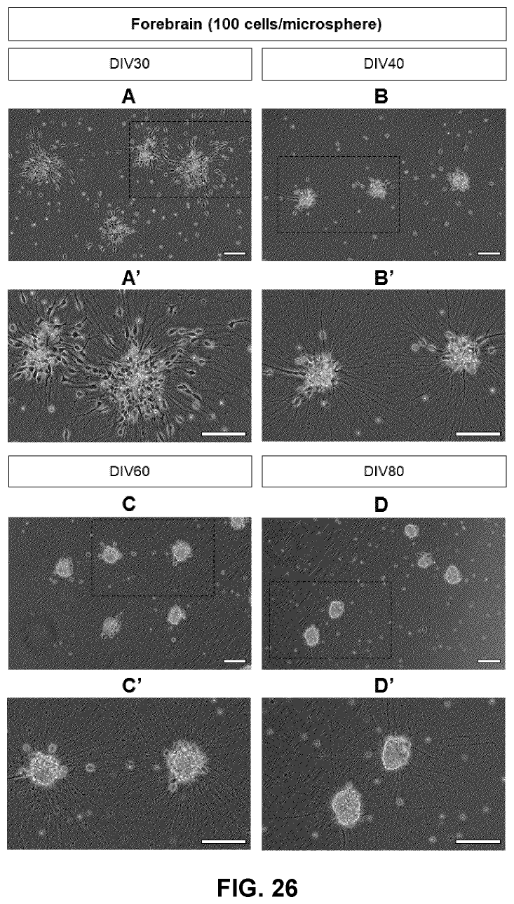

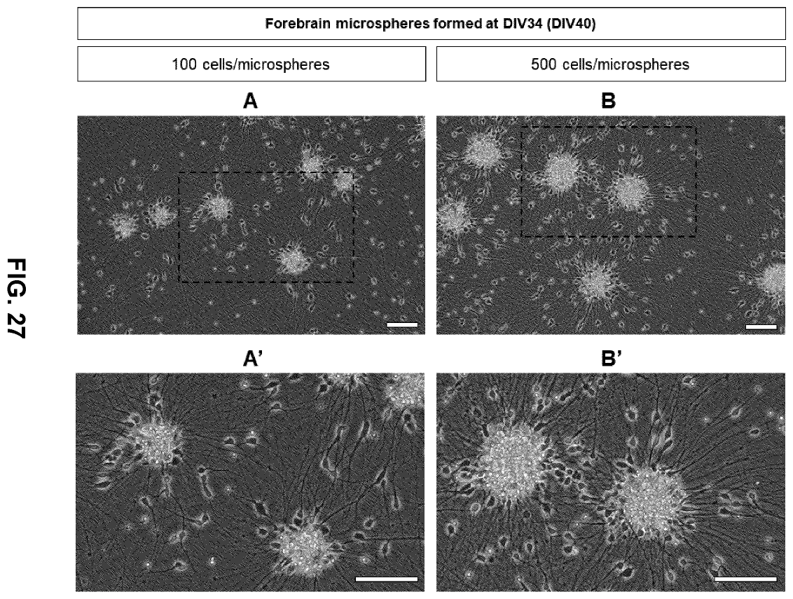

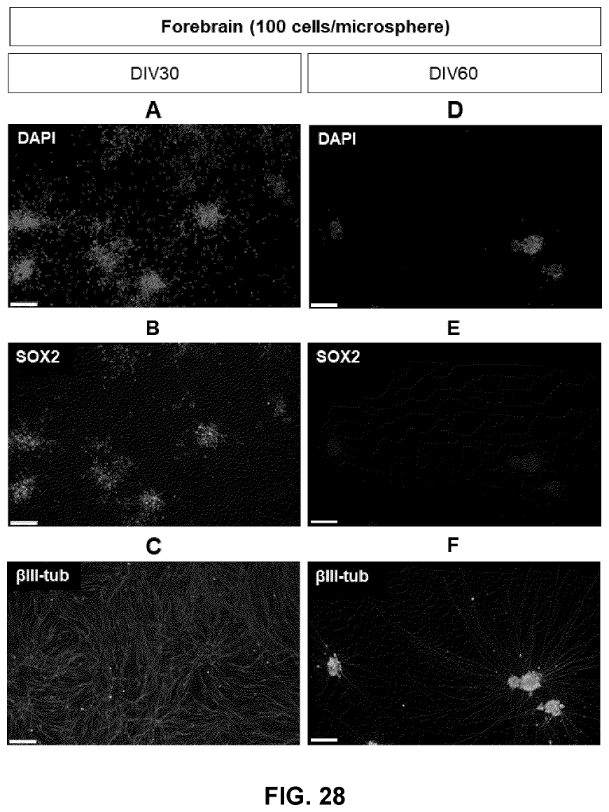

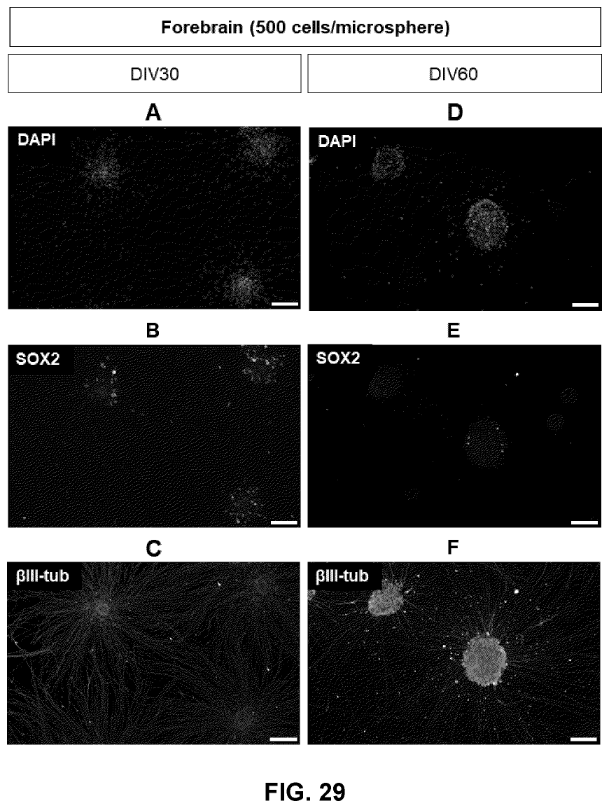

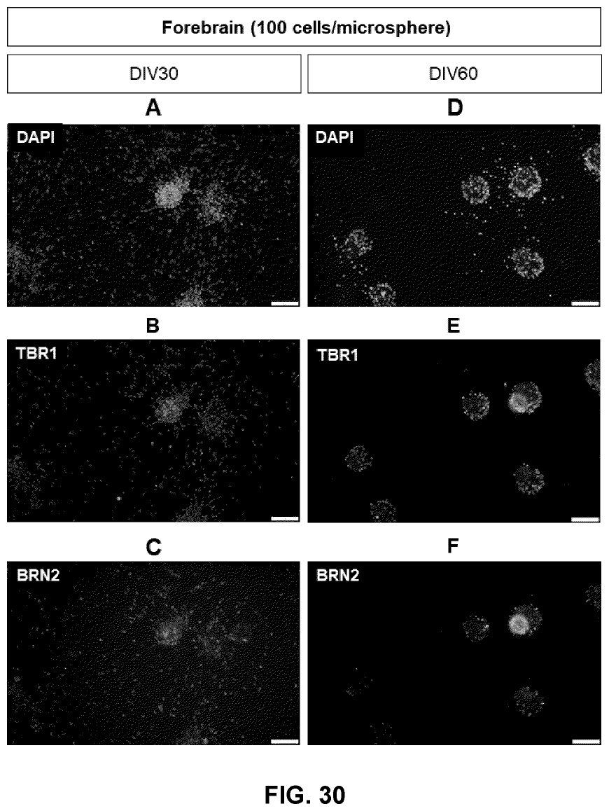

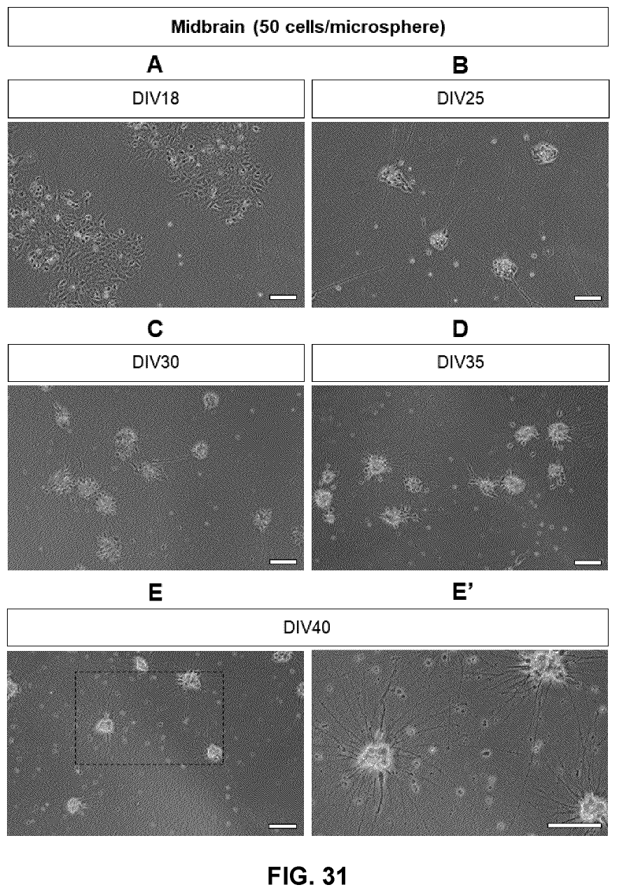

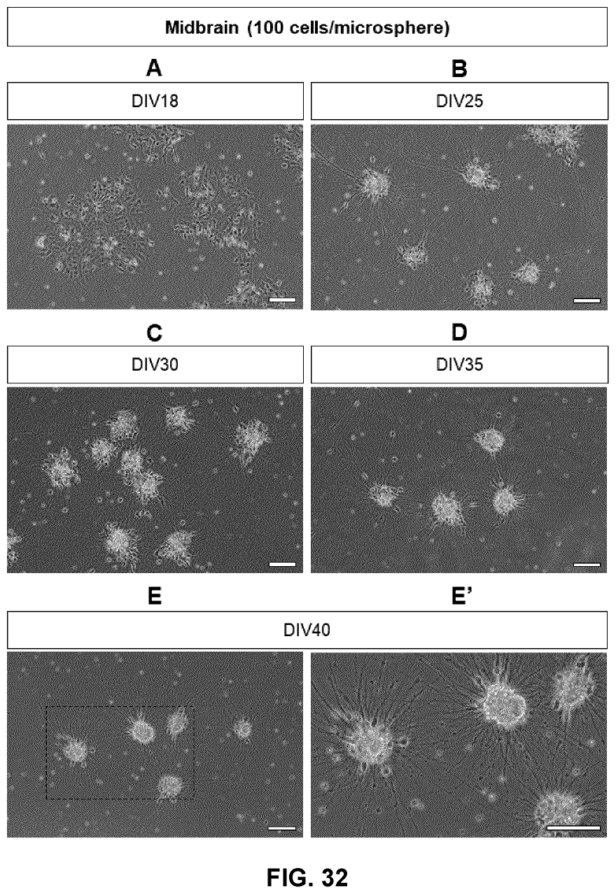

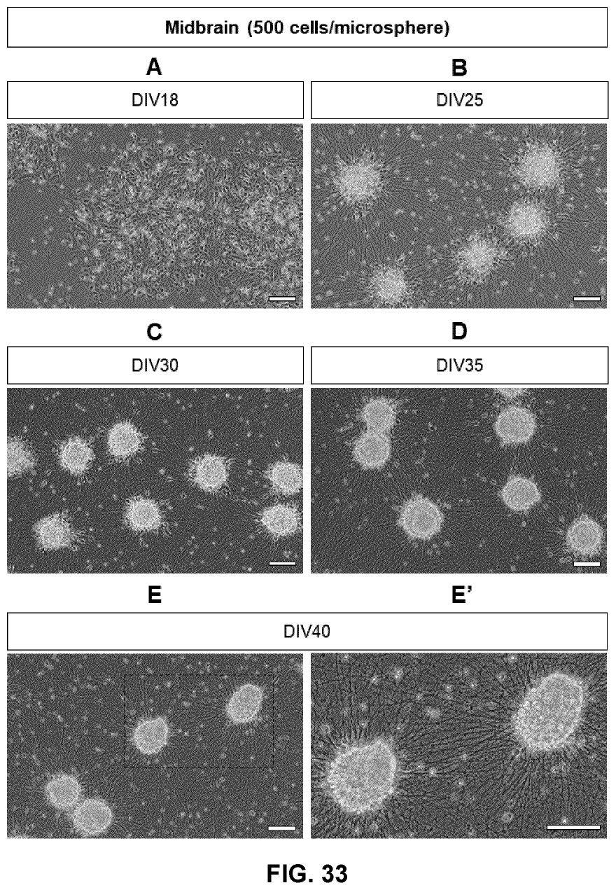

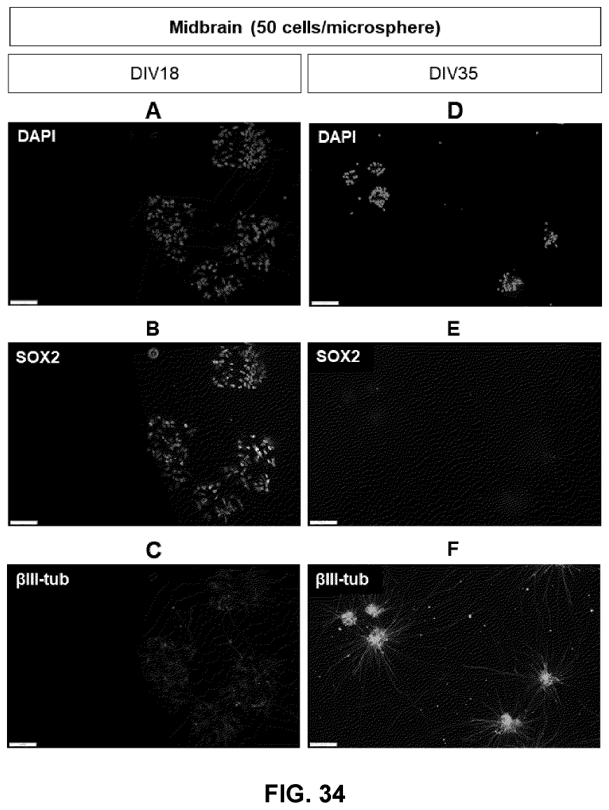

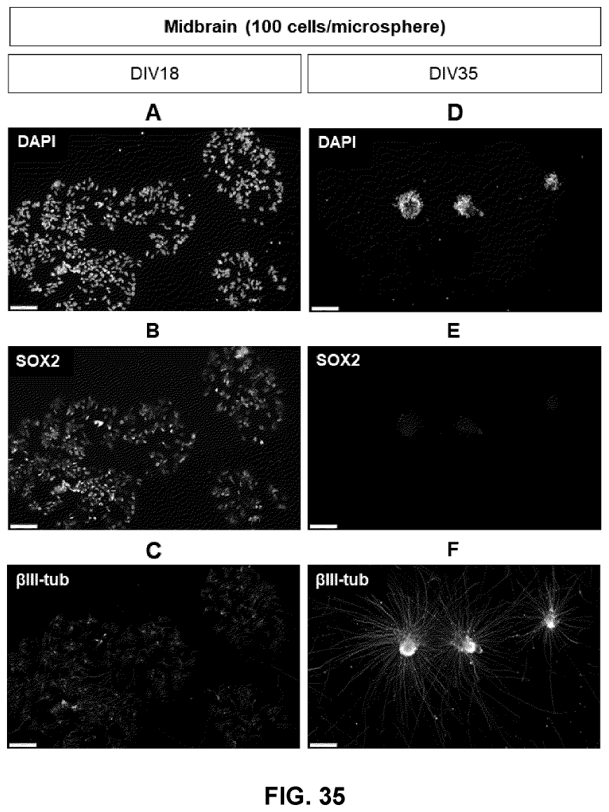

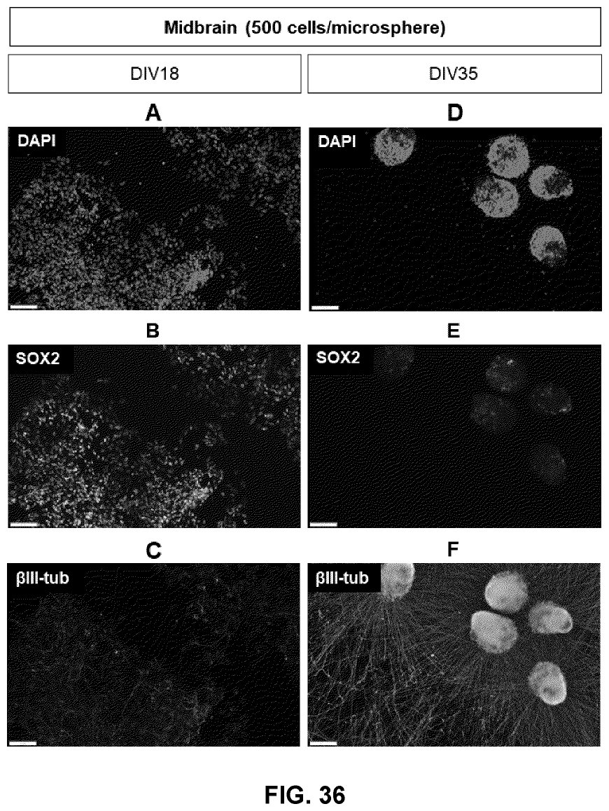

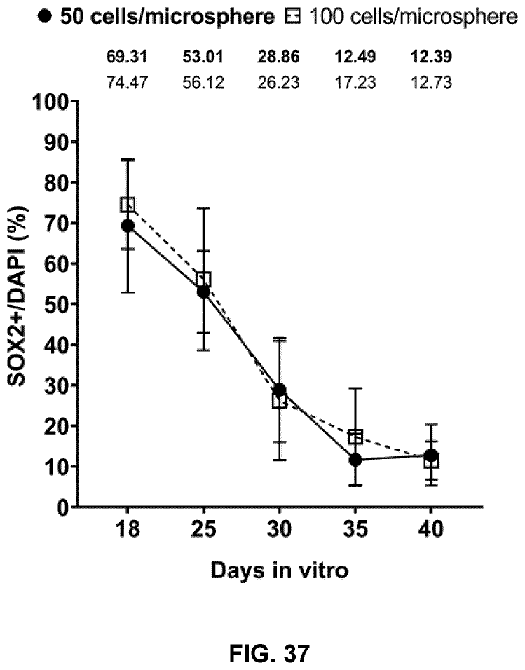

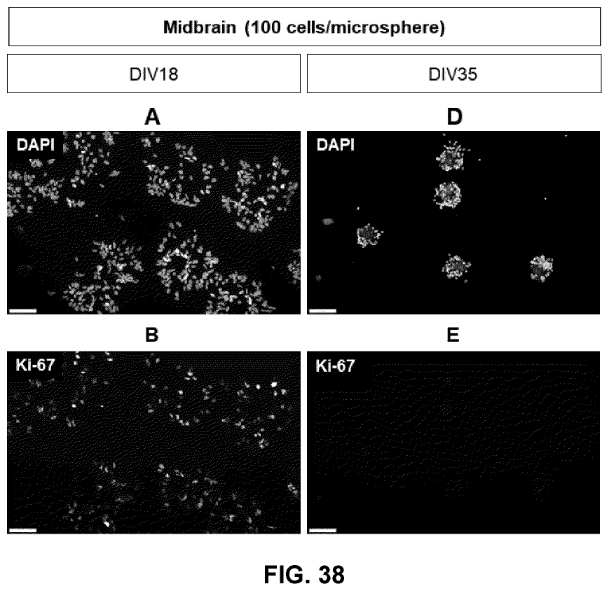

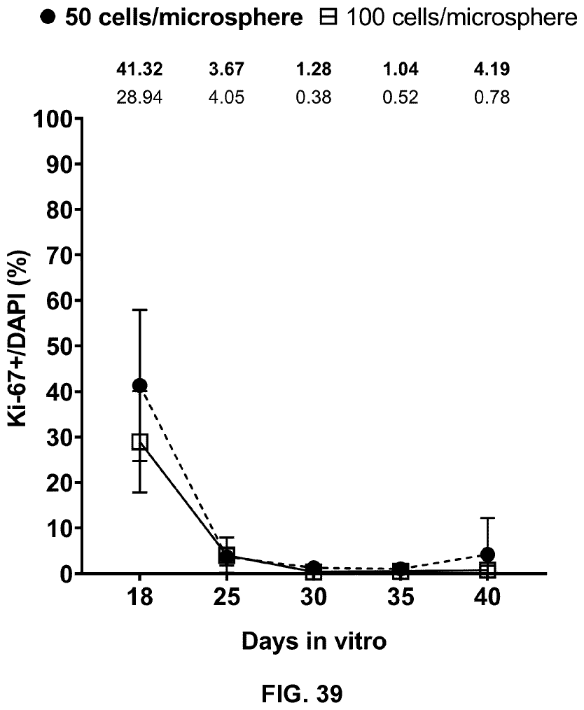

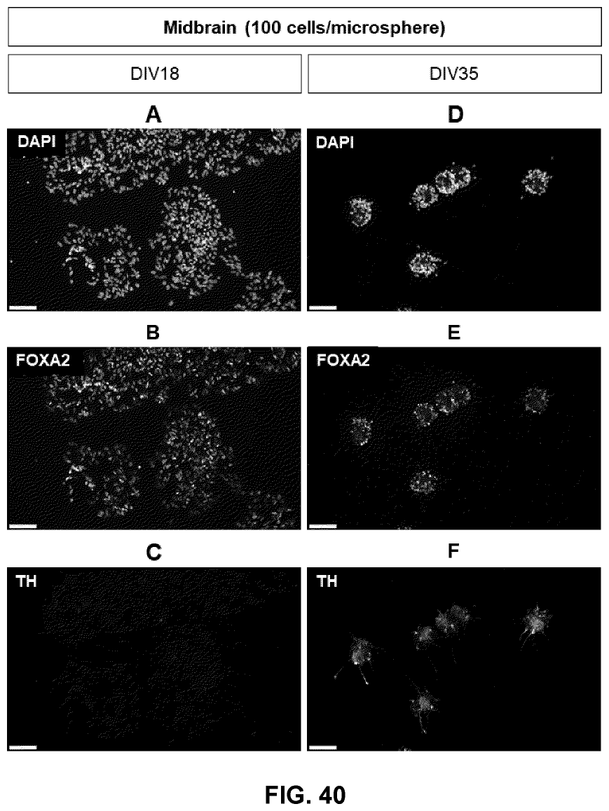

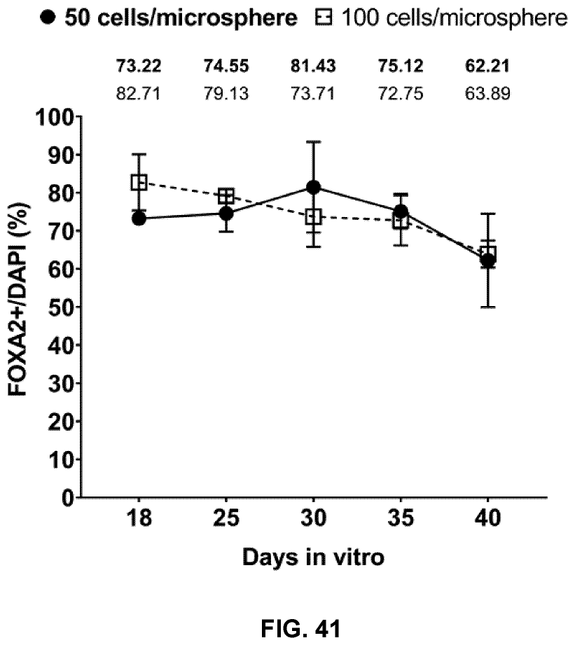

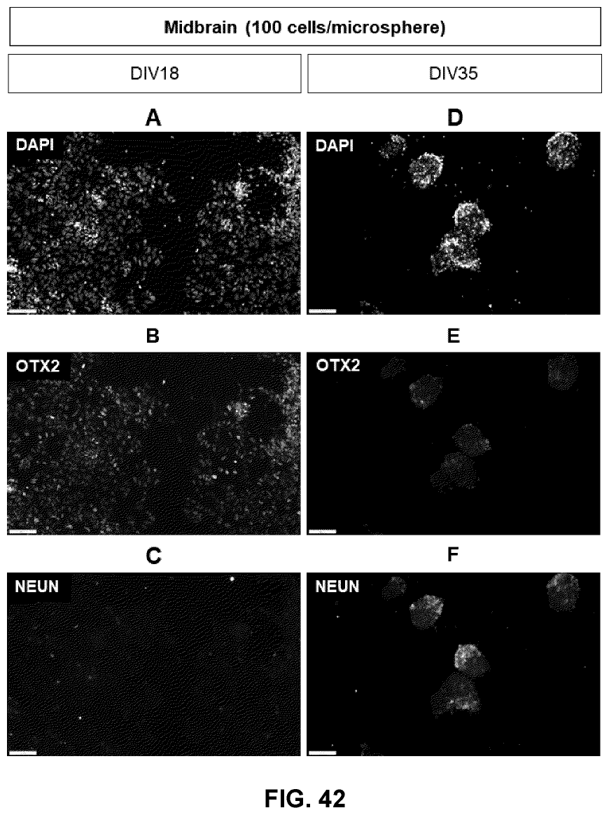

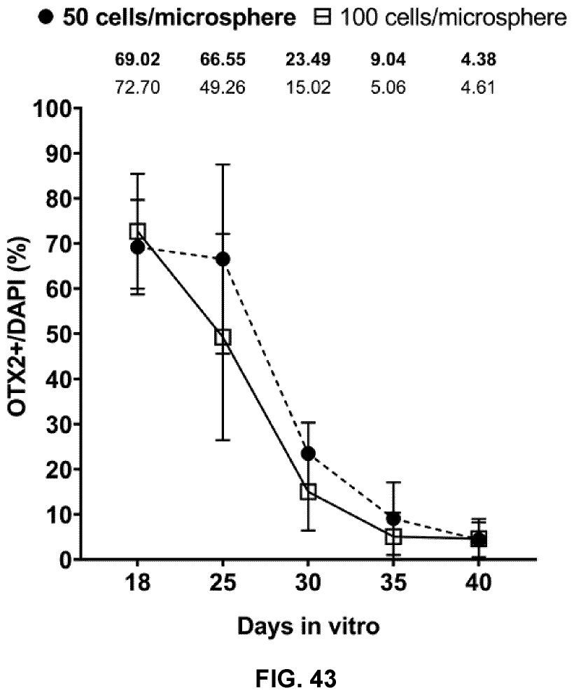

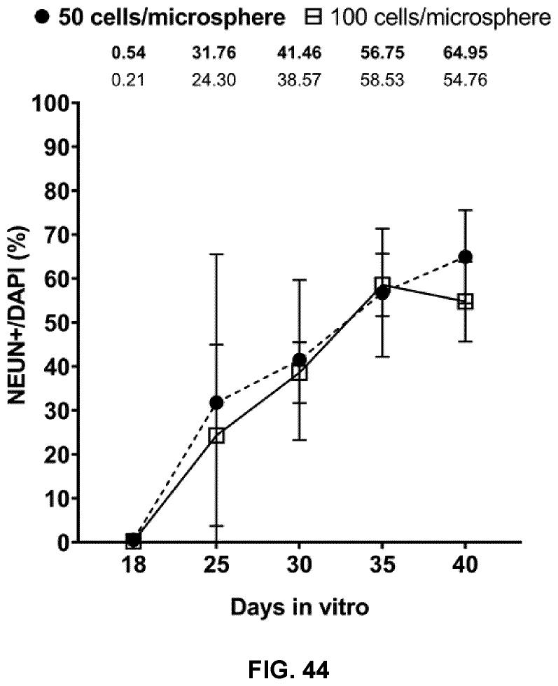

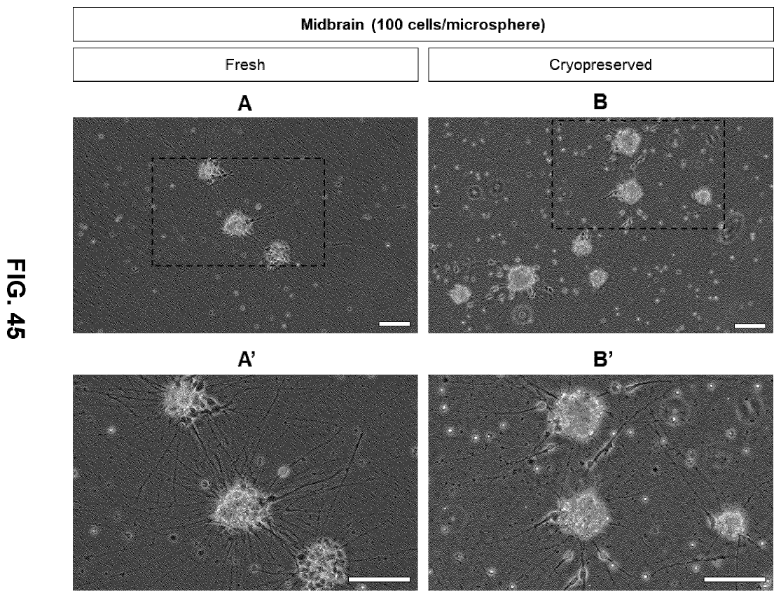

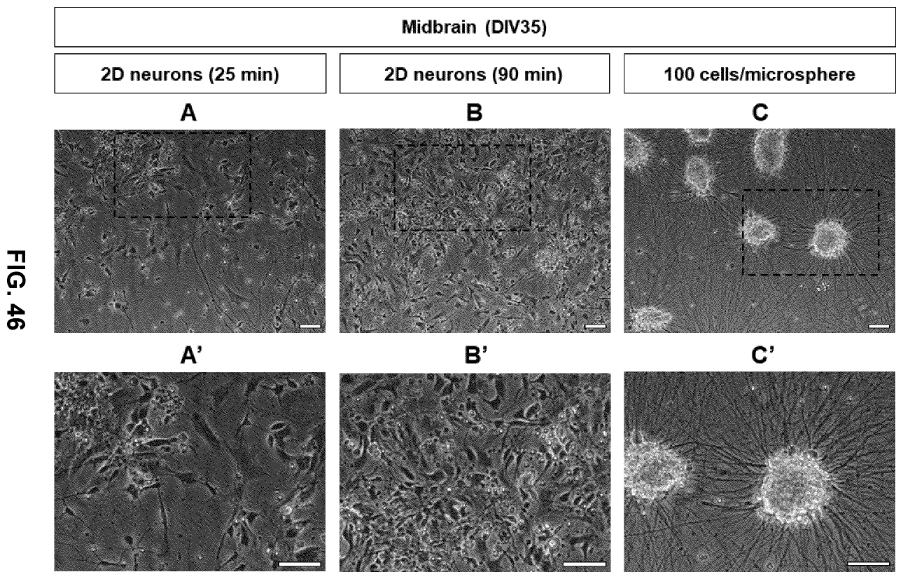

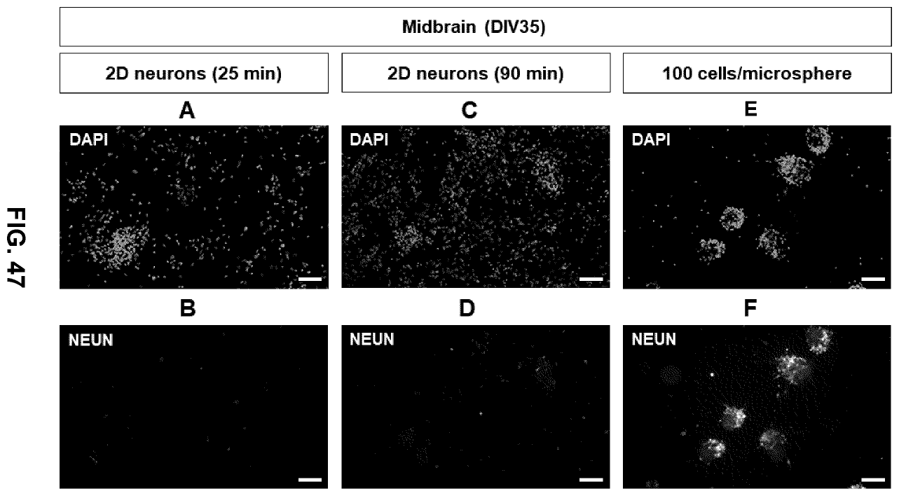

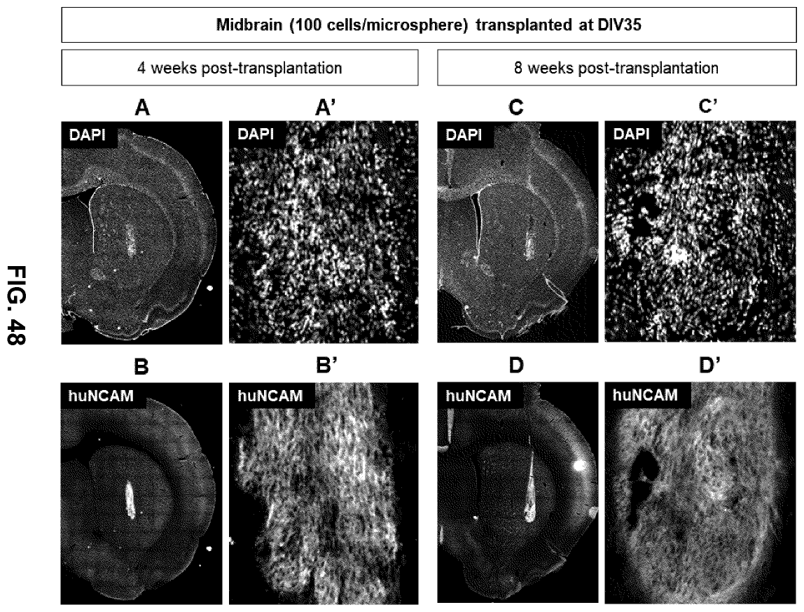

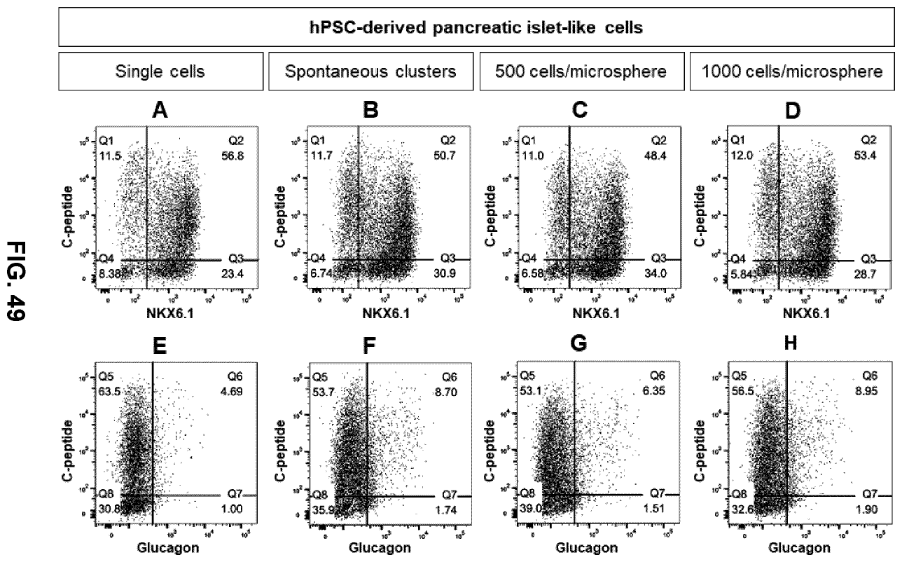

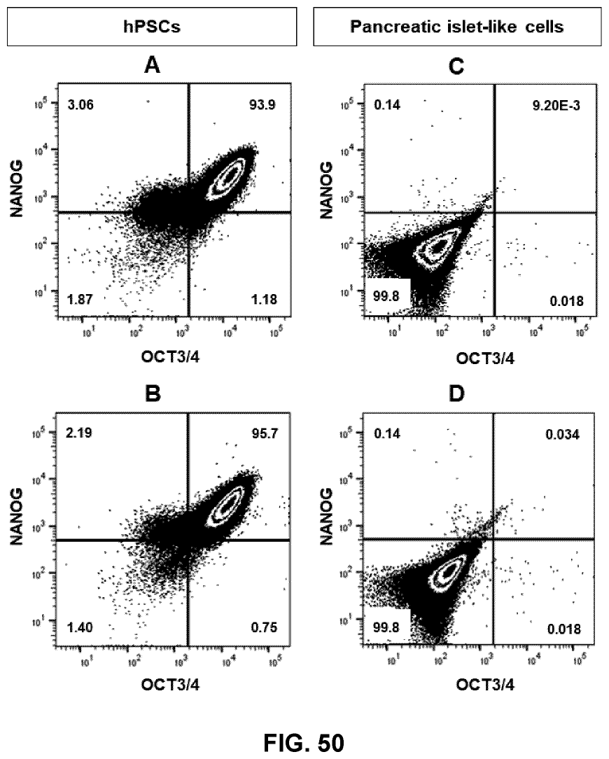

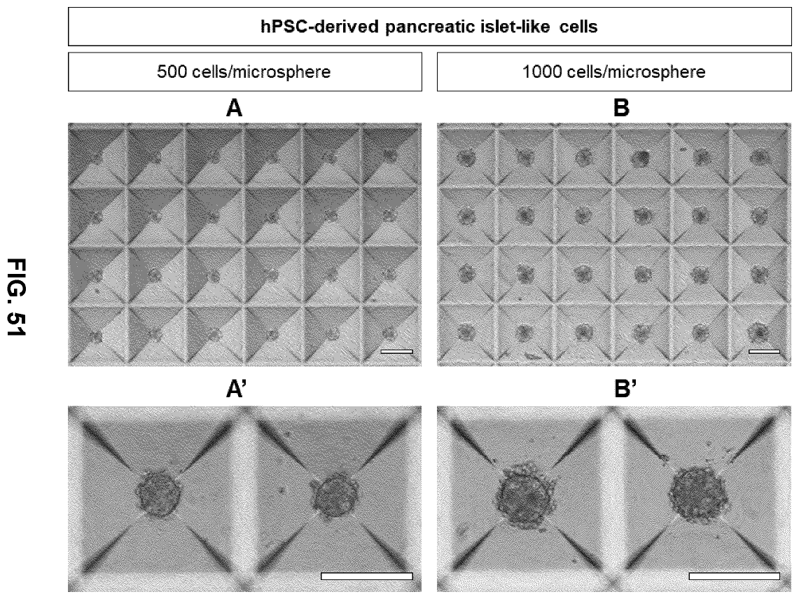

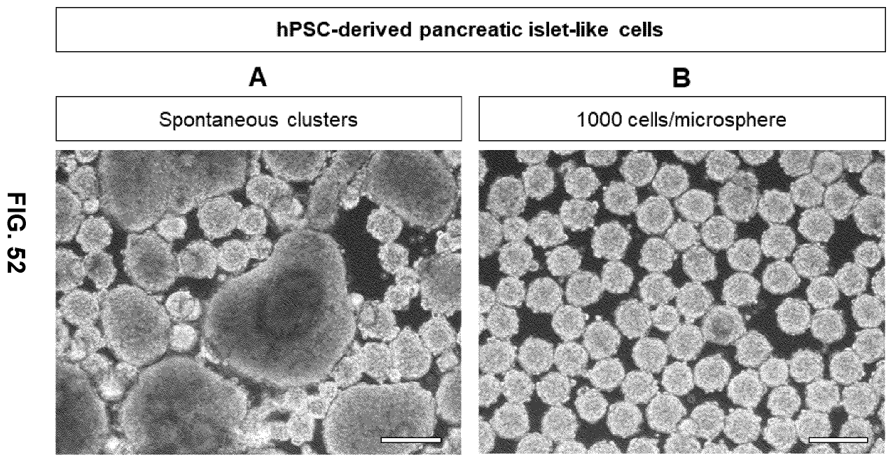

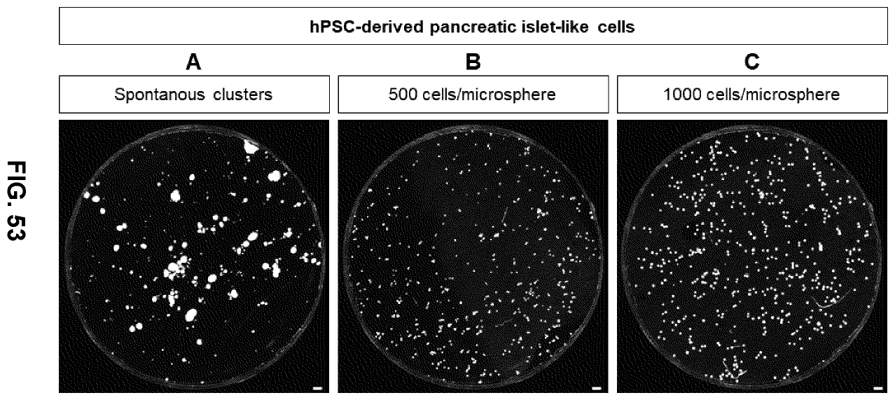

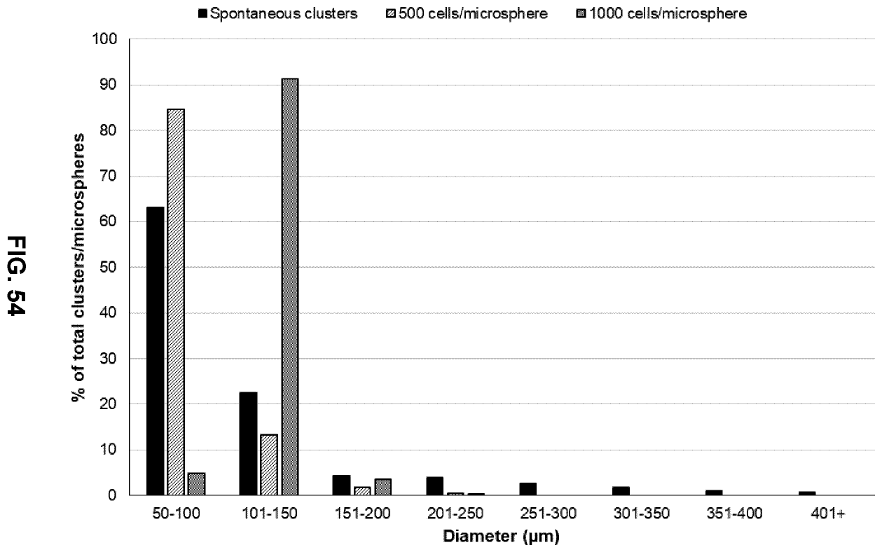

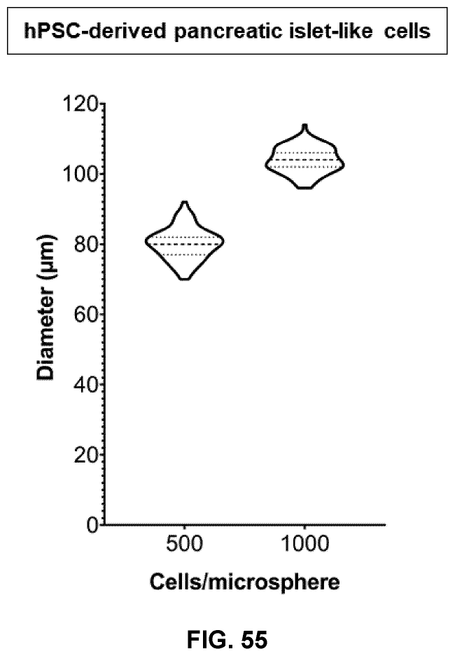

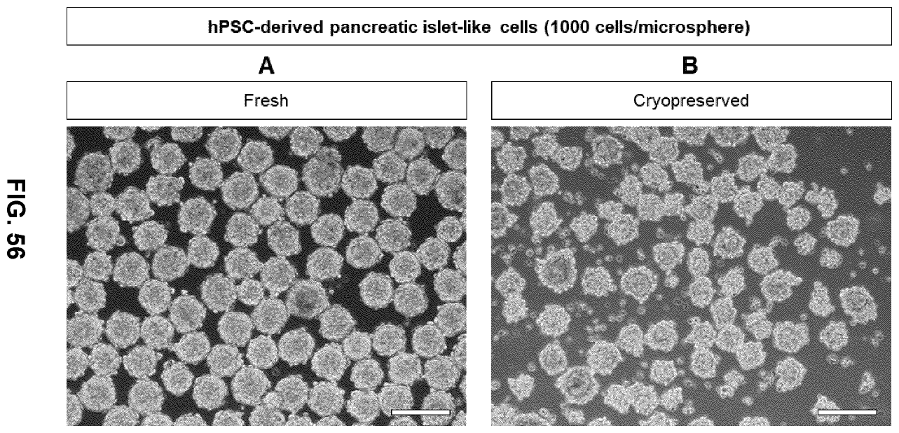

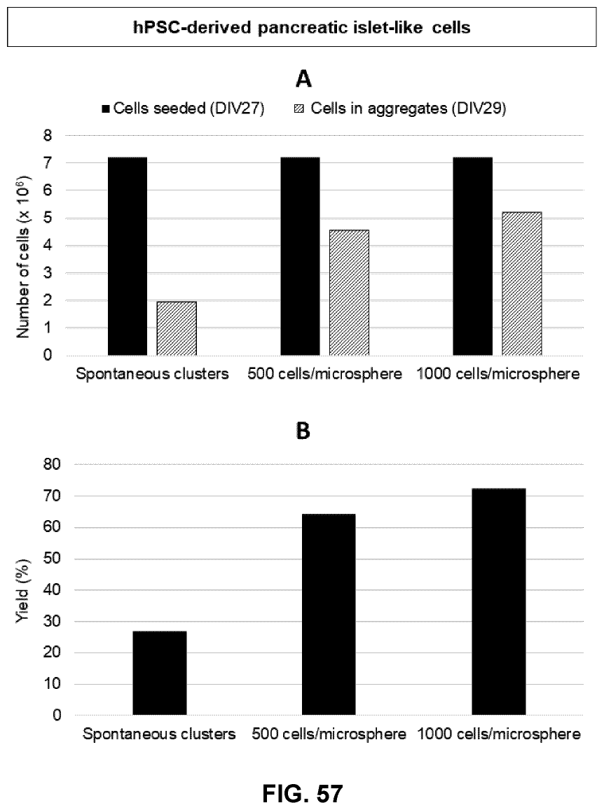

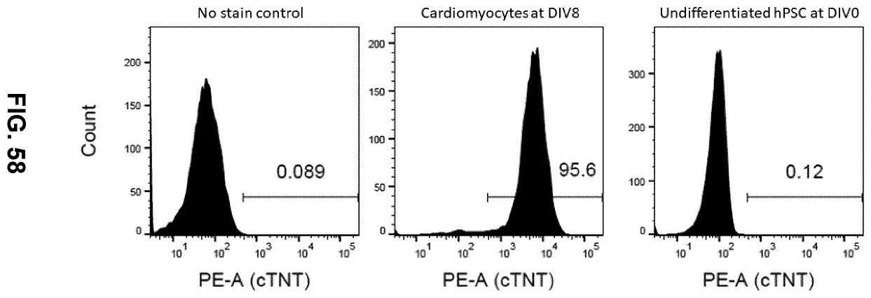

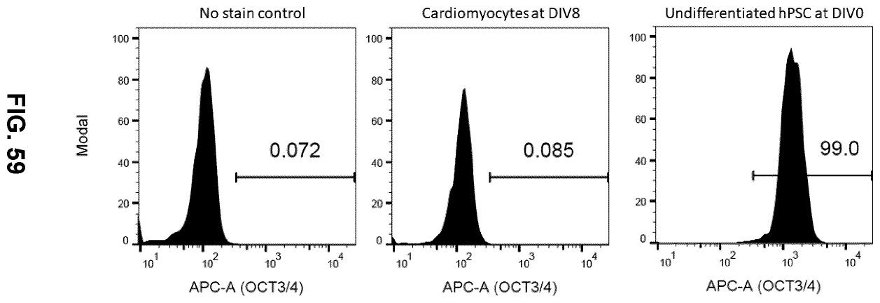

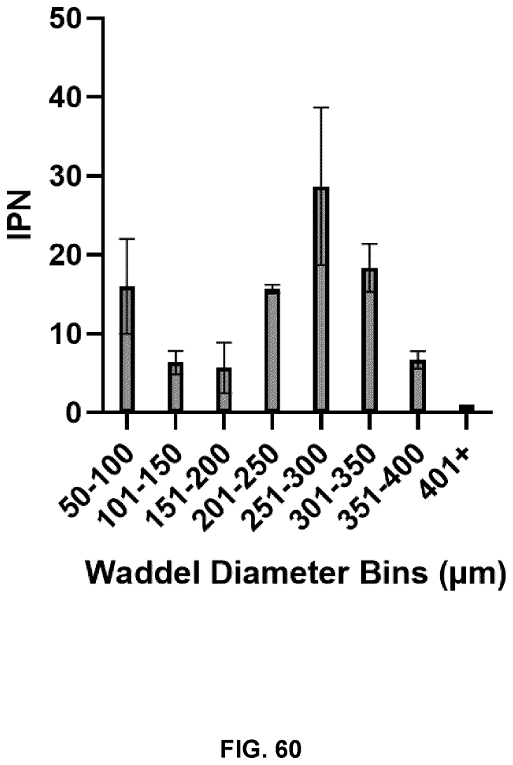

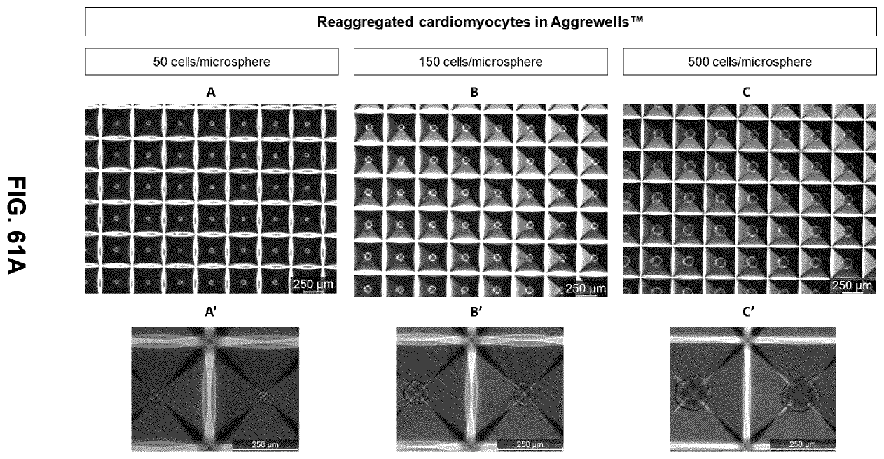

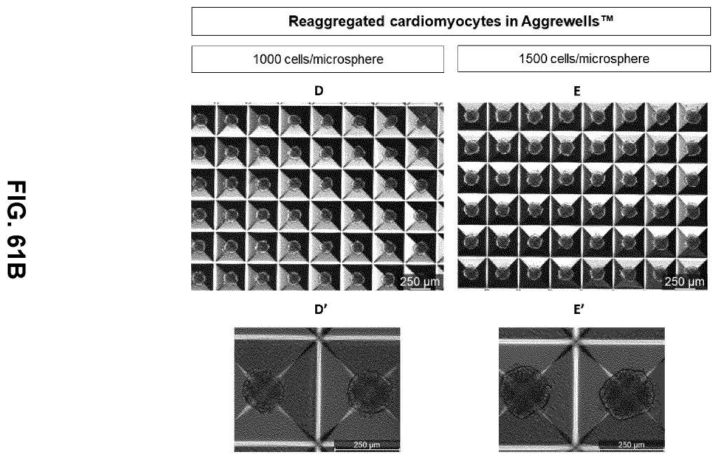

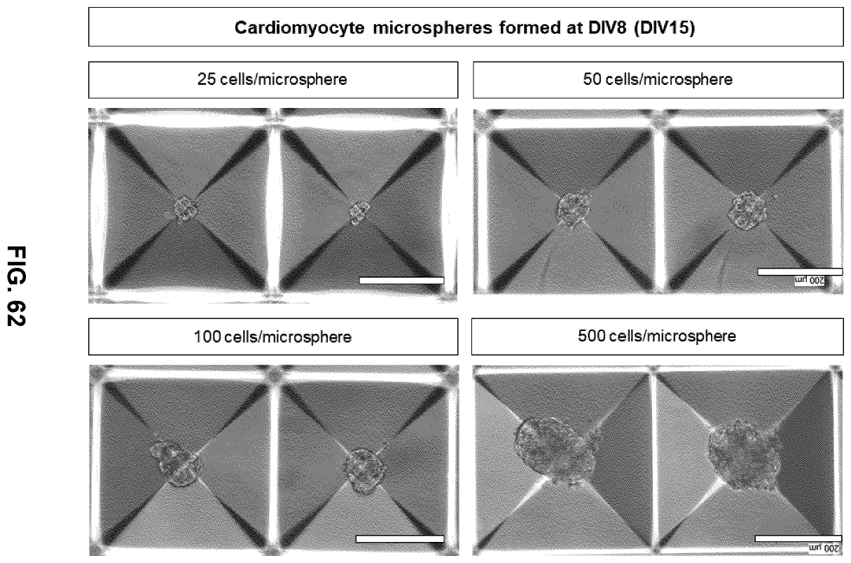

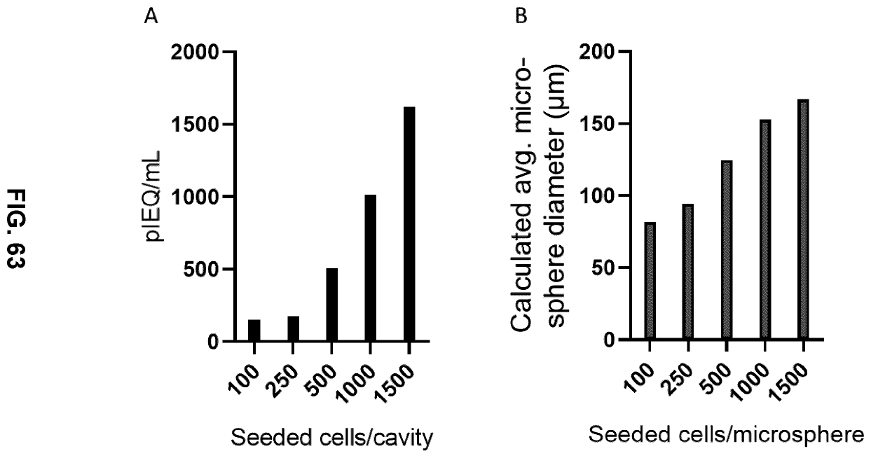

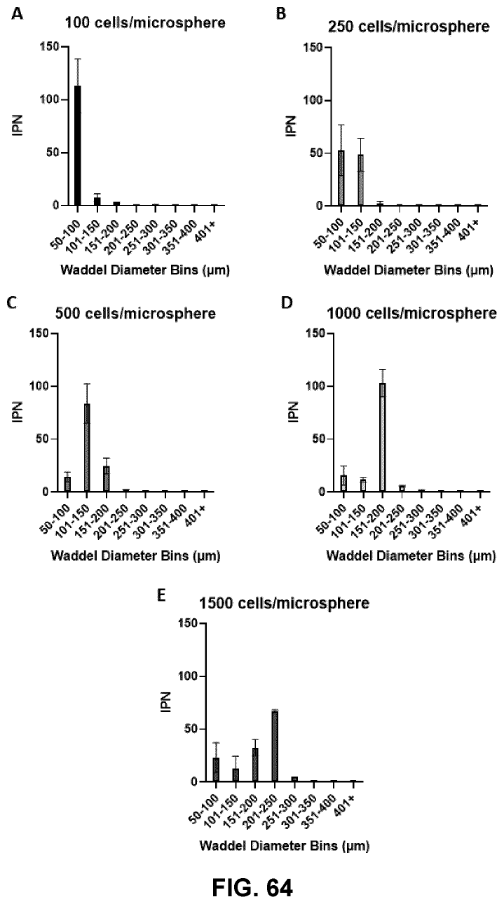

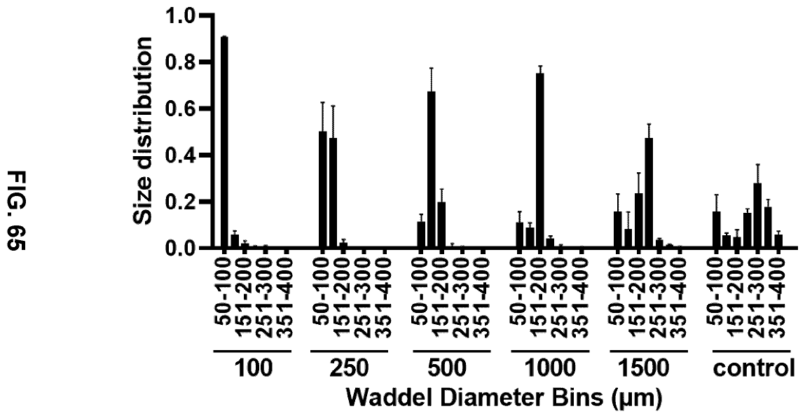

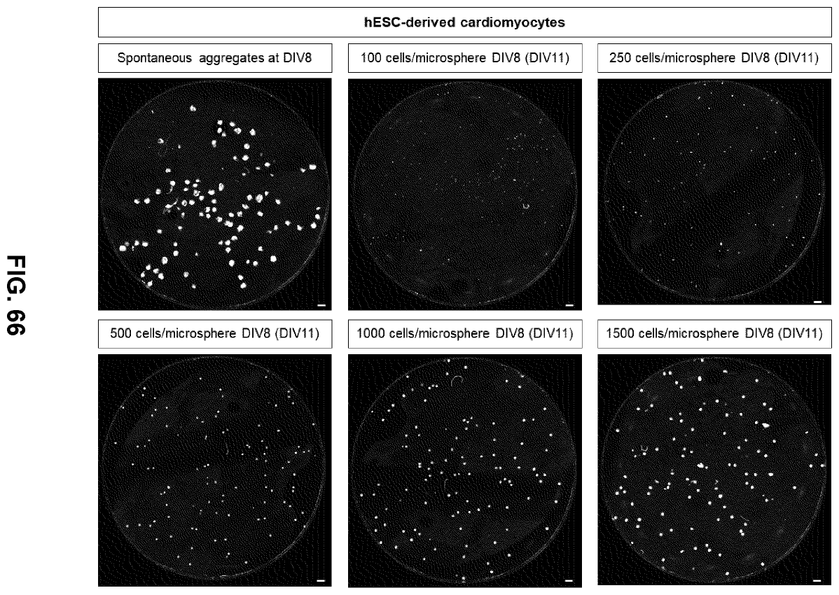

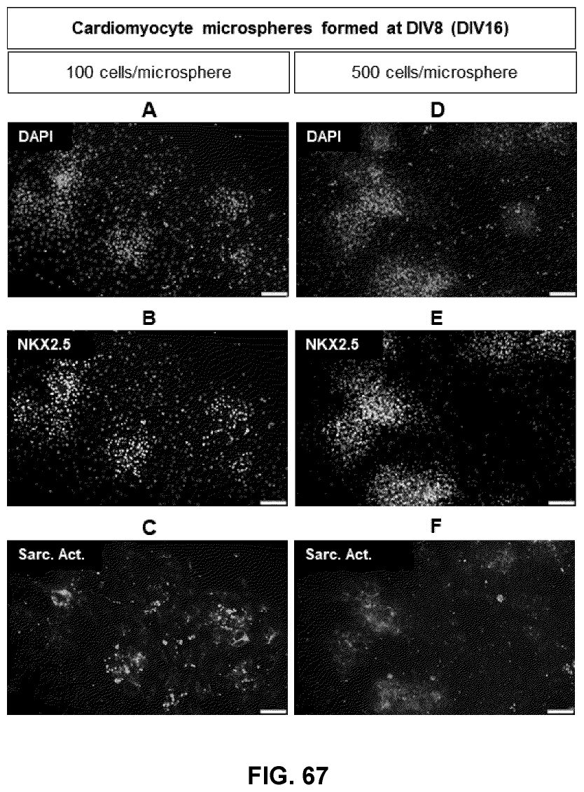

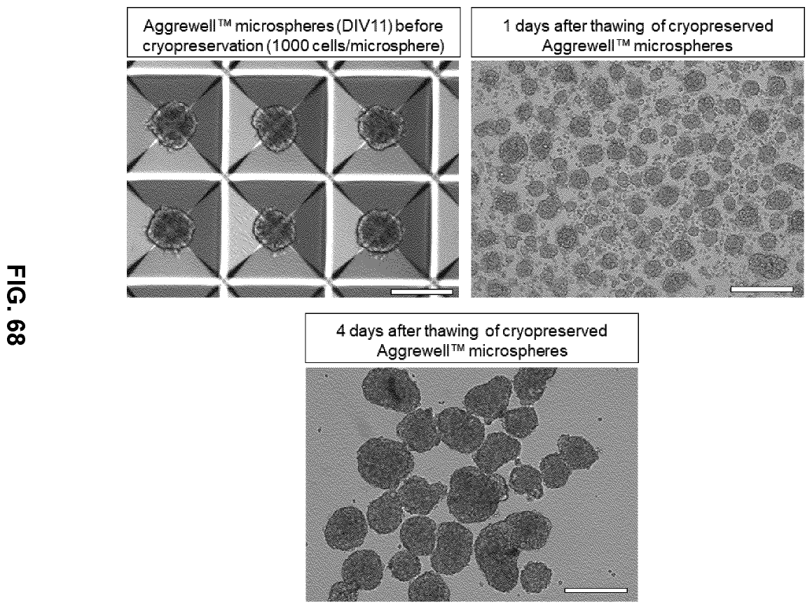

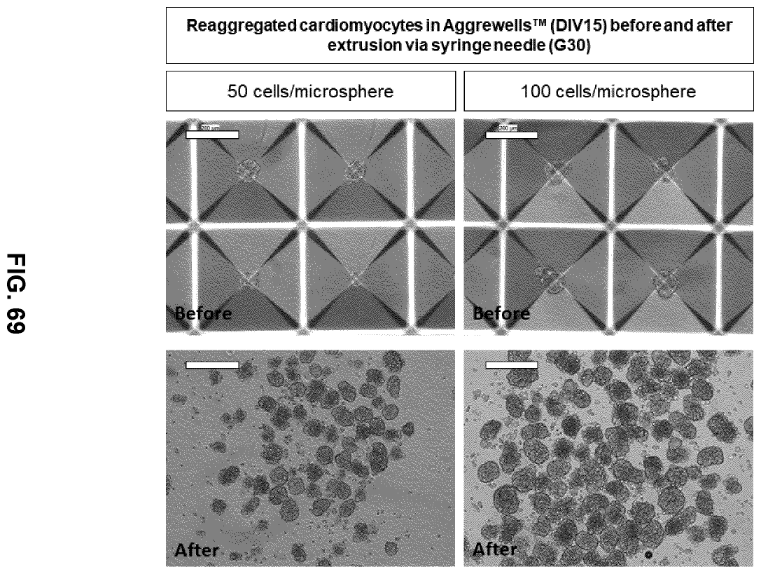

NSPCs analysed at DIV19 by flow cytometry using antibodies against SOX2 (A), OTX2 (A, B), PAX6 (B, C), SOX1 (C), OCT3/4 (D), and Nanog (D). The numbers represent the percentage of events in each quadrant. FIG. 3 shows a bar graph summarising the percentage of cells expressing the markers PAX6, OTX2, SOX1, and SOX2 (mean+SD) as determined by flow cytometry across 2-5 dorsal forebrain differentiations. FIG. 4 shows representative phase-contrast images of dorsal forebrain NSPCs (A) and neurons matured in a standard 2D format (B). FIG. 5 shows representative immunofluorescence images of ventral midbrain NSPCs stained for DAPI (A), FOXA2 (B), and OTX2 (C) at DIV16. Scale bar: 100 μm. FIG. 6 shows representative histogram (A) and dot plots (B, C) of ventral midbrain NSPCs analysed at DIV16 by flow cytometry using antibodies against SOX2 (A), FOXA2 (B), OTX2 (B, C), and PAX6 (C). The numbers in (A) show the percentages of SOX2 negative and positive events. The numbers in (B) and (C) represent the percentage of events in each quadrant. FIG. 7 shows representative dot plots of hPSCs (A) and ventral midbrain NSPCs (B) analysed by flow cytometry using antibodies against the pluripotency markers OCT3/4 and Nanog. The numbers represent the percentage of events in each quadrant. FIG. 8 shows venn diagrams obtained from single cell RNA sequencing (scRNA-seq) analysis of ventral midbrain NSPCs at DIV 16. The venn diagrams show the percentage of cells expressing one or more of the genes SOX2, NES, MK167, and DCX (A) or POU5F1 (i.e. OCT4), Nanog, CD9, and PODXL1 (B). FIG. 9 shows the expression of the genes SOX2 and ASCL1 int-SNE plots (A, C) and venn diagrams (B, D) obtained from scRNA-seq analysis of ventral midbrain NSPCs at DIV 16 and NBCs at DIV26. FIG. 10 shows representative immunofluorescence images of ventral midbrain NSPCs at DIV16 (A, B) and neurons matured in a standard 2D format until DIV40 (C-F) stained for DAPI (A, C, E), beta-III tubulin (B, D), and tyrosine hydroxylase (F). Scale bar: 100 μm. FIG. 11 shows representative immunofluorescence images of ventral hindbrain/spinal cord NSPCs stained for DAPI (A, D), NKX6.1 (B), and OTX2 (D) at DIV14. Scale bar: 100 μm. FIG. 12 shows representative dot plots of ventral hindbrain/spinal cord NSPCs analysed at DIV19 by flow cytometry using antibodies against SOX2 (A), OTX2 (A, B), and NKX6.1 (B). The numbers represent the percentage of events in each quadrant. FIG. 13 shows the relative expression of the CNS lineage-specific genes EN1, FOXA2, LMX1A, OTX2, NKX6.1, and PAX6 across hPSCs (black bars) and hPSC-derived dorsal forebrain (FB, striated bars), ventral midbrain (MB, dotted bars), and ventral hindbrain/spinal cord (HB-SC, white bars) NSPCs as determined by nanostring analysis.

FIGS. 14-22 show examples of neural microsphere formation and non-adherent static culture. FIG. 14 shows a schematic illustration of the neural microsphere formation (A) and static non-adherent culture (B) procedures as well as potential applications including seeding and adherent culture in vitro (C), cryopreservation (D), and transplantation (E). FIG. 15 shows representative low (A, B) and high (A′, B′) magnification phase-contrast images of microspheres inside microwells two days after formation. The microspheres were made from 100 (A) and 500 (B) dorsal forebrain NSPBCs at DIV28. Scale bar: 200 μm. FIG. 16 shows representative phase-contrast images of microspheres inside microwells 32 days after formation. The microspheres were made from 100 (A) and 500 (B) dorsal forebrain NSPBCs at DIV28. Scale bar: 200 μm. FIG. 17 shows representative low (A, B) and high (A′, B′) magnification phase-contrast images of microspheres inside microwells six days after formation. The microspheres were made from 100 (A) and 500 (B) dorsal forebrain NSPBCs at DIV34. Scale bar: 200 μm. FIG. 18 shows representative low (A-D) and high (A′-D′) magnification phase-contrast images of microspheres inside microwells two days after formation. The microspheres were made from 50 (A), 100 (B), 500 (C) and 150 (D) ventral midbrain NSPBCs at DIV16 either by spin-aggregation (A-C) or by letting cells sediment by gravity alone without centrifugation into microwells (D). Scale bar: 200 μm. FIG. 19 shows representative low (A-C) and high (A′-C′) magnification phase-contrast images of microspheres inside microwells 24 days after formation. The microspheres were made from 50 (A), 100 (B), and 500 (C) ventral midbrain NSPBCs at DIV16. Scale bar: 200 μm. FIG. 20 shows representative phase-contrast images of microspheres inside microwells three days after formation. The microspheres were made from 100 (A) and 500 (B) ventral midbrain NSPBCs at DIV26. Scale bar: 200 μm. FIG. 21 shows representative low (A, B) and high (A′, B′) magnification phase-contrast images of microspheres inside microwells ten days after formation. The microspheres were made from 100 (A) and 500 (B) ventral midbrain NSPBCs at DIV26. Scale bar: 200 μm. FIG. 22 shows representative low (A) and high (A′) magnification phase-contrast images of microspheres inside microwells two days after formation. The microspheres were made from 100 ventral hindbrain/spinal cord NSPBCs at DIV20. Scale bar: 200 μm.

FIGS. 23-25 show neural microsphere size measurements. FIG. 23 shows violin plots demonstrating variation in diameter of microspheres made from 100 or 500 forebrain NSPBCs across two different experiments (A) and maturation stages (B). FIG. 24 shows violin plots demonstrating the variation in diameter of microspheres made from 50, 100 , or 500 midbrain NSPBCs across three different experiments (A) and two maturation stages (B). FIG. 25 shows a violin plot demonstrating the variation in diameter of microspheres made from 100 hindbrain/spinal cord NSPBCs.

FIGS. 26-44 show the composition and maturation of neural microspheres at different stages of static non-adherent culture. FIG. 26 shows representative low (A-D) and high (A′-D′) magnification phase-contrast images of microspheres 48 hours after seeding onto a laminin substrate and cultured in neuronal maturation media. The microspheres were made from 100 forebrain NSPBCs at DIV28and seeded at DIV30 (A, A′), DIV40 (B, B′), DIV60 (C, C′), and DIV80 (D, D′). Scale bar: 100 μm. FIG. 27 shows representative low (A, B) and high (A′, B′) magnification phase-contrast images of microspheres 48 hours after seeding onto a laminin substrate and cultured in neuronal maturation media. The microspheres were made from 100 or 500 forebrain NSPBCs at DIV34 and seeded at DIV40. Scale bar: 100 μm. FIG. 28 shows representative immunofluorescence images of microspheres made from 100 forebrain NSPBCs, seeded onto a laminin substrate on DIV30 (A-C) or DIV60 (D-F), and stained for DAPI (A, D), SOX2 (B, E), and beta-III tubulin (C, F). Scale bar: 100 μm. FIG. 29 shows representative immunofluorescence images of microspheres made from 500 forebrain NSPBCs, seeded onto a laminin substrate on DIV30 (A-C) or DIV60 (D-F), and stained for DAPI (A, D), SOX2 (B, E), and beta-III tubulin (C, F). Scale bar: 100 μm. FIG. 30 shows representative immunofluorescence images of microspheres made from 100 forebrain NSPBCs, seeded onto a laminin substrate on DIV30 (A-C) or DIV60 (D-F), and stained for DAPI (A, D), TBR1 (B, E), and BRN2 (C, F). Scale bar: 100 μm. FIG. 31 shows representative low (A-E) and high (E′) magnification phase-contrast images of microspheres 48 hours after seeding onto a laminin substrate and cultured in neuronal maturation media. The microspheres were made from 50 midbrain NSPBCs at DIV16 and seeded at DIV18 (A), DIV25 (B), DIV30 (C), DIV35 (D), and DIV40 (E, E′). Scale bar: 100 μm. FIG. 32 shows representative low (A-E) and high (E′) magnification phase-contrast images of microspheres 48 hours after seeding onto a laminin substrate and cultured in neuronal maturation media. The microspheres were made from 100 midbrain NSPBCs at DIV16 and seeded at DIV18 (A), DIV25 (B), DIV30 (C), DIV35 (D), and DIV40 (E, E′). Scale bar: 100 μm. FIG. 33 shows representative low (A-E) and high (E′) magnification phase-contrast images of microspheres 48 hours after seeding onto a laminin substrate and cultured in neuronal maturation media. The microspheres were made from 500 midbrain NSPBCs at DIV16 and seeded at DIV18 (A), DIV25 (B), DIV30 (C), DIV35 (D), and DIV40 (E, E′). Scale bar: 100 μm. FIG. 34 shows representative immunofluorescence images of microspheres made from 50 midbrain NSPBCs, seeded onto a laminin substrate on

DIV18 (A-C) or DIV35 (D-F), and stained for DAPI (A, D), SOX2 (B, E), and beta-III tubulin (C, F). Scale bar: 100 μm. FIG. 35 shows representative immunofluorescence images of microspheres made from 100 midbrain NSPBCs, seeded onto a laminin substrate on DIV18 (A-C) or DIV35 (D-F), and stained for DAPI (A, D), SOX2 (B, E), and beta-III tubulin (C, F). Scale bar: 100 μm. FIG. 36 shows representative immunofluorescence images of microspheres made from 500 midbrain NSPBCs, seeded onto a laminin substrate on DIV18 (A-C) or DIV35 (D-F), and stained for DAPI (A, D), SOX2 (B, E), and beta-III tubulin (C, F). Scale bar: 100 μm. FIG. 37 shows the percentage of SOX2 positive cells (mean±SD) within microspheres made from either 50 (black circles, bold font) or 100 (white squares, regular font) midbrain NSPBCs seeded at DIV18, DIV25, DIV30, DIV35, or DIV40 and cultured for 48 hours. The numbers listed above the graph represent the mean values. FIG. 38 shows representative immunofluorescence images of microspheres made from 100 midbrain NSPBCs, seeded onto a laminin substrate on DIV18 (A, B) or DIV35 (D, E), and stained for DAPI (A, D) and Ki-67 (B, E). Scale bar: 100 μm. FIG. 39 shows the percentage of Ki-67 positive cells (mean±SD) within microspheres made from either 50 (black circles, bold font) or 100 (white squares, regular font) midbrain NSPBCs seeded at DIV18, DIV25, DIV30, DIV35, or DIV40 and cultured for 48 hours. The numbers listed above the graph represent the mean values. FIG. 40 shows representative immunofluorescence images of microspheres made from 100 midbrain NSPBCs, seeded onto a laminin substrate on DIV18 (A, B) or DIV35 (D, E), and stained for DAPI (A, D), FOXA2 (B, E), and tyrosine hydroxylase (C, F). Scale bar: 100 μm. FIG. 41 shows the percentage of FOXA2 positive cells (mean±SD) within microspheres made from either 50 (black circles, bold font) or 100 (white squares, regular font) midbrain NSPBCs seeded at DIV18, DIV25, DIV30, DIV35, or DIV40 and cultured for 48 hours. The numbers listed above the graph represent the mean values. FIG. 42 shows representative immunofluorescence images of microspheres made from 100 midbrain NSPBCs, seeded onto a laminin substrate on DIV18 (A, B) or DIV35

(D, E), and stained for DAPI (A, D), OTX2 (B, E), and NEUN (C, F). Scale bar: 100 μm. FIG. 43 shows the percentage of OTX2 positive cells (mean±SD) within microspheres made from either 50 (black circles, bold font) or 100 (white squares, regular font) midbrain NSPBCs seeded at DIV18, DIV25, DIV30, DIV35, or DIV40 and cultured for 48 hours. The numbers listed above the graph represent the mean values. FIG. 44 shows the percentage of NEUN positive cells (mean±SD) within microspheres made from either 50 (black circles, bold font) or 100 (white squares, regular font) midbrain NSPBCs seeded at DIV18, DIV25, DIV30, DIV35, or DIV40 and cultured for 48 hours. The numbers listed above the graph represent the mean values.

FIG. 45 shows representative low (A, B) and high (A′, B′) magnification phase-contrast images of midbrain microspheres before (A, A′) and after (B, B′) cryopreservation. The microspheres were either seeded fresh at DIV35 or cryopreserved at DIV35, seeded upon thaw, and cultured for 48 hours. Scale bar: 100 μm.

FIGS. 46-48 show preparation and transplantation of neural microspheres. FIG. 46 shows representative low (A-C) and high (A′-C′) magnification phase-contrast images of midbrain cells passed through thinly-pulled glass capillaries at DIV35 mimicking the transplantation procedure, seeded onto a laminin substrate, and cultured for 48 hours. The cells were either obtained from standard 2D neuronal cultures dissociated with accutase for 25 min. (A, A′) or 90 min. (B, B′) in order to obtain a single cell suspension mimicking the standard procedure for transplantation of NSPBCs, or intact microspheres made from 100 midbrain NSPBCs. Scale bar: 100 μm. FIG. 47 shows representative immunofluorescence images of midbrain cells passed through thinly-pulled glass capillaries at DIV35 mimicking the transplantation procedure, seeded onto a laminin substrate, cultured for 48 hours, and stained for DAPI (A, C, E) and NEUN (B, D, F). The cells were either obtained from standard 2D neuronal cultures dissociated with accutase for 25 min. (A, A′) or 90 min. (B, B′) in order to obtain a single cell suspension mimicking the standard procedure for transplantation of NSPBCs, or intact microspheres made from 100 midbrain NSPBCs. Scale bar: 100 μm. FIG. 48 shows images of rat brain coronal sections obtained 4 weeks (A, A′, B, B′) and 8 weeks (C, C′, D, D′) after intra-striatal transplantation of hPSC-derived microspheres made from 100 midbrain NSPBCs. The sections were stained by immunohistochemistry for DAPI (A, A′, C, C′) and human-specific NCAM (B, B′, D, D′). Low magnification images (A-D) show the ispilateral hemisphere and high magnification images (A′-D′) show the grafted area.

FIGS. 49-57 show examples of microspheres comprised of hPSC-derived pancreatic islet-like cells. FIG. 49 shows representative dot plots of hPSC-derived pancreatic islet-like cells analysed by flow cytometry using antibodies against NKX6.1 (A-D), glucagon (E-H), and c-peptide (A-H). The numbers represent the percentage of events in each quadrant. The cells were analysed as a single cell suspension prior to aggregate formation on DIV29 (A, E) and after formation of spontaneous clusters (B, F) and microspheres (C, D, G, H) FIG. 50 shows representative dot plots of hPSCs (A) and hPSC-derived pancreatic islet-like cells (B) analysed by flow cytometry at DIV25 (C) and DIV29 (D) using antibodies against the pluripotency markers OCT3/4 and Nanog. The numbers represent the percentage of events in each quadrant. FIG. 51 shows representative low (A, B) and high (A′, B′) magnification phase-contrast images of microspheres inside microwells two days after formation. The microspheres were made from 500 (A) and 1000 (B) hPSC-derived pancreatic islet-like cells. Scale bar: 200 pm. FIG. 52 shows representative phase-contrast images of clusters formed by spontaneous aggregation of hPSC-derived pancreatic islet-like cells in suspension culture (A) and microspheres formed by controlled spin-aggregation of 1000 hPSC-derived pancreatic islet-like cells (B). Scale bar: 200 μm. FIG. 53 shows mask images from biorep analysis of clusters formed by spontaneous aggregation of hPSC-derived pancreatic islet-like cells in suspension culture (A) and microspheres formed by controlled spin-aggregation of 500 (B) or 1000 (C) hPSC-derived pancreatic islet-like cells (B). Scale bar: 500 μm. FIG. 54 shows the size distribution determined by biorep analysis of spontaneously aggregated clusters (black bars) and microspheres made from either 500 (striated bars) or 1000 (dotted bars) hPSC-derived pancreatic islet-like cells. FIG. 55 shows violin plots demonstrating variation in diameter of microspheres made from 500 or 1000 hPSC-derived pancreatic islet-like cells 48 hours after microsphere formation. FIG. 56 shows representative phase-contrast images of microspheres before (A) and after (B) cryopreservation. The microspheres were made from 1000 hPSC-derived pancreatic islet-like cells. Scale bar: 200 μm. FIG. 57 shows bar graphs representing the number of seeded cells integrated into aggregates either as controlled microspheres or spontaneously formed clusters in total numbers (A) and percent yield (B).

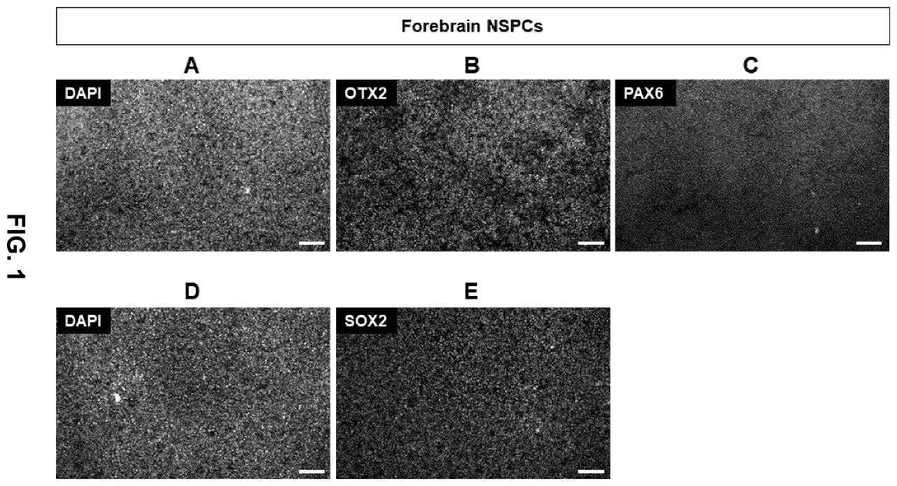

FIGS. 58-69 show examples of microspheres comprised of hPSC-derived cardiomyocyte-like cells. FIG. 58 shows representative histograms of hPSC-derived cardiomyocyte-like cells and undifferentiated hPSCs analysed by flow cytometry using antibodies against cardiac Troponin T. The cardiomyocyte-like cells were obtained from 3D aggregates on day 8 of differentiation (DIV8) and analysed as single cell suspension prior to microsphere formation. FIG. 59 shows representative histograms of hPSC-derived cardiomyocyte-like cells and undifferentiated hPSCs analysed by flow cytometry using antibodies against Oct3/4. The cells were analysed as a single cell suspension obtained from 3D aggregates on day 8 (DIV8) prior to microsphere formation and day 0 of differentiation (DIV0), respectively. FIG. 60 shows representative size distribution of cardiomyocyte-like cells obtained on day 8 of the differentiation (DIV8) by automated cluster analysis. FIGS. 61-1 and 61-2 shows representative low (A, B, C, D, E) and high (A′, B′, C′, D′, E′) magnification phase-contrast images of microspheres inside microwells two days after formation (DIV10). The microspheres were made from cryopreserved hPSC-derived cardiomyocytes cells (DIV8) with the following cell numbers per cluster: 50 (A, A′), 150 (B, B′), 500 (C, C′), 1000 (D, D′) or 1500 (E, E′). Scale bar: 250 μm. FIG. 62 shows representative magnification phase-contrast images of microspheres inside microwells 7 days after formation (DIV15). The microspheres were made from 25, 50, 100 and 500 hPSC-derived cardiomyocyte-like cells. Scale bar: 200 μm. FIG. 63 shows the cell mass displayed as plEQ/mL (A) and the average sphere diameter (B) determined by automated biorep analysis from microspheres formed by controlled spin-aggregation of either 100, 250, 500, 1000 or 1500 hPSC-derived cardiomyocyte-like cells. FIG. 64 shows the absolute particle count for indicated diameter bins based on 200 μl sample volume determined by automated biorep analysis for microspheres made from either 100 (A), 250 (B), 500 (C), 1000 (D) or 1500 (E) hPSC-derived cardiomyocyte-like cells two days after aggregation (DIV10). FIG. 65 shows the relative size distribution of microspheres formed from each measurement 100, 250, 500, 1000 or 1500 hPSC-derived cardiomyocyte-like cells two days after aggregation (DIV10). FIG. 66 shows mask images from biorep analysis of spontaneously formed aggregates of hPSC-derived cardiomyocyte-like cells from the 3D suspension culture process (DIV8) and microspheres formed by controlled spin-aggregation of 100, 250, 500, 1000 or 1500 hPSC-derived cardiomyocyte-like cells. Scale bar: 500 μm. FIG.67 shows representative microspheres of 100 (A, B, C) and 500 (D, E, F) cells plated on Laminin 521 for 24 h and stained against NKX2.5 (B, E) and Sarcomeric Actinin (C, F). The nuclei were counterstained using DAPI (A, D). Scale bar: 200 μm. FIG. 68 shows phase-contrast images of microspheres comprising pluripotent stem-cell derived cardiomyocyte before and after cryopreservation. Microspheres were formed from 1000 cells per cavity from cryopreserved single cells (DIV8) and cryopreserved after three days (DIV11). Scale bar: 250 82 m. FIG. 69 shows phase-contrast images of microspheres before and after extrusion via a syringe needle (G30). Microspheres were formed from 50 or 100 cells per cavity on day 8 (DIV8) and harvested as well extruded 7 days after microsphere formation (DIV15). Scale bar: 200 μm.

### DESCRIPTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed.

### General Definitions

Throughout this application the terms “method” and “protocol” when referring to processes for differentiating cells may be used interchangeably. As used herein, “a” or “an” or “the” can mean one or more than one. Unless otherwise indicated in the specification, terms presented in singular form also include the plural situation. As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

As used herein, the term “day” and similarly day in vitro (DIV) in reference to the protocols refers to a specific time for carrying out certain steps.

In general and unless otherwise stated “day 0” refers to the initiation of the protocol, this be by for example but not limited to plating the stem cells or transferring the stem cells to an incubator or contacting the stem cells in their current cell culture medium with a compound prior to transfer of the stem cells. Typically, the initiation of the protocol will be by transferring undifferentiated stem cells to a different cell culture medium and/or container such as but not limited to by plating or incubating, and/or with the first contacting of the undifferentiated stem cells with a compound that affects the undifferentiated stem cells in such a way that a differentiation process is initiated.

When referring to “day X”, such as day 1, day 2 etc., it is relative to the initiation of the protocol at day 0. One of ordinary skill in the art will recognize that unless otherwise specified the exact time of the day for carrying out the step may vary. Accordingly, “day X” is meant to encompass a time span such as of +/−10 hours, +/−8 hours, +/−6 hours, +/−4 hours, +/−2 hours, or +/−1 hours.

As used herein, the phrase “from at about day X to at about day Y” refers to a day at which an event starts from. The phrase provides an interval of days on which the event may start from. For example, if “cells are contacted with a differentiating factor from at about day 3 to at about day 5” then this is to be construed as encompassing all the options: “the cells are contacted with a differentiating factor from about day 3”, “the cells are contacted with a differentiating factor from about day 4”, and “the cells are contacted with a differentiating factor from about day 5”. Accordingly, this phrase should not be construed as the event only occurring in the interval from day 3 to day 5. This applies mutatis mutandis to the phrase “to at about day X to at about day Y”.

Hereinafter, the methods according to the present invention are described in more detail by non-limiting embodiments and examples. A method is provided for obtaining neural microspheres comprising stem cell-derived neural cells. Accordingly, the method take offset in the use of stem cells.

By “stem cell” is to be understood an undifferentiated cell having differentiation potency and proliferative capacity (particularly self-renewal competence) but maintaining differentiation potency. The stem cell includes subpopulations such as pluripotent stem cell, multipotent stem cell, unipotent stem cell and the like according to the differentiation potency.

As used herein, the term “pluripotent stem cell” (PSC) refers to a stem cell capable of being cultured in vitro and having a potency to differentiate into any cell lineage belonging to three germ layers (ectoderm, mesoderm, endoderm) and/or extraembryonic tissue (pluripotency). As used herein, the term “multipotent stem cell” means a stem cell having a potency to differentiate into plural types of tissues or cells, though not all kinds. As used herein, the term “unipotent stem cell” means a stem cell having a potency to differentiate into a particular tissue or cell. A pluripotent stem cell can be induced from fertilized egg, clone embryo, germ stem cell, stem cell in a tissue, somatic cell and the like. Examples of the pluripotent stem cell (PSC) include embryonic stem cell (ESC), EG cell (embryonic germ cell), induced pluripotent stem cell (iPSC) and the like. Muse cell (Multi-lineage differentiating stress enduring cell) obtained from mesenchymal stem cell (MSC), and GS cell produced from reproductive cell (e.g., testis) are also encompassed in the pluripotent stem cell. As used herein, the term “induced pluripotent stem cell” (also known as iPS cells or iPSCs) means a type of pluripotent stem cell that can be generated directly from adult cells. By the introduction of products of specific sets of pluripotency-associated genes adult cells can be converted into pluripotent stem cells.

Embryonic stem cells may also be derived from parthenotes as described in e.g. WO 2003/046141. Additionally, embryonic stem cells can be produced from a single blastomere or by culturing an inner cell mass obtained without the destruction of the embryo. Embryonic stem cells are available from given organizations and are also commercially available. Preferably, the methods and products of the present invention are based on hPSCs, i.e. stem cells derived from either induced pluripotent stem cells or embryonic stem cells, including parthenotes. As used herein, the term “stem cell” is also meant to embrace cells obtained by direct conversion (also termed transdifferentiation) or forward programming where any cell type is converted to neural stem progenitor cells (NSPCs) via overexpression of identity and master regulator genes or delivery of specific small molecules that switch on identity and master regulator genes. As used herein, the term “compact and moveable formats” typically refers to cultures disassociated to a single cell suspension or small clusters of few cells, a procedure only applied to NSPBCs, or microspheres themselves.

Protocol for Obtaining Neural Microspheres Derived from Stem Cells

In a general aspect of the present invention is provided a method for obtaining a neural microsphere, comprising the steps of providing neural stem precursor blast cells, aggregating the neural stem precursor blast cells to form a neural microsphere, and allowing the neural stem precursor blast cells of the neural microsphere to further mature. In an embodiment, the step of providing neural stem precursor blast cells comprises the step of differentiating PSCs into neural stem precursor blast cells. In an embodiment, the step of providing neural stem precursor blast cells further comprises the initial step of culturing PSCs.

According to the present invention a “neural microsphere” is defined as a cell organization forming a three-dimensional cluster comprising neural cells. By the term “microsphere” is meant a sphere-like structure formed by cells substantially having a defined boundary. A person skilled in the art will readily recognize that a cluster of cells inherently will never form a perfectly round geometrical shape in the three-dimensional space. The cluster may for example be elongated in spheroid-like structure or protrude or depress in certain areas. The terms “cluster” and “aggregate” may be used interchangeably and refer to multiple cells having grouped together so that adjacent cells are in close proximity and/or direct contact with each other, and wherein the adjacent cells have an affinity towards each other so as to maintain the three-dimensional structure of the cluster. By the term “microsphere” is further meant that the cluster comprises a number of cells forming a sphere-like structure with a diameter measured in micrometer, such as less than 350 μm.

As used herein, the term “neural” refers to the nervous system. As used herein, the term “neural cell” refers to cells mimicking a cell type, which are naturally part of the ectoderm germ layer, more specifically the neuroectoderm and is meant to encompass cells at any stage of development within this germ layer, such as neural stem cells all the way through to neurons, i.e. cell stages such as neural stem progenitor cell stage and neural blast cell stage. Accordingly, specific embodiments referring to neural cells may apply equally to embodiments comprising e.g. only neural stem progenitor cells or only neural blast cells or a mixture thereof. Neural cells may be derived from embryonic stem cells and induced pluripotent stem cells, and other pluripotent cells (i.e. parthenogenic stem cells) and via direct conversion (also referred to transdifferentiation) methodologies.

As used herein, the term “neural stem progenitor cell” (NSPC) refers to cells having the capacity to self-renew, proliferate and/or differentiate into one or more than one cell type. Neural progenitor cells can therefore be unipotent, bipotent or multipotent. Neural stem progenitor cells (NSPCs) are therefore mitotic and typically differentiate terminally into neurons and glial cells as well as other cell types that reside in the CNS including meningeal cells.

As used herein, the term “neural blast cell” (NBC) refers to neural cells that are further differentiated than neural stem progenitor cells (NSCPs), typically have the capacity to further differentiate (i.e. to neurons) and do not self-renew and are postmitotic.

The term “neural stem precursor blast cell” (NSPBC) is here within used to collectively describe a pure population of either NSCs, neural precursor cells or neural progenitor cells (collectively referred to as NPCs) with NSC and NPCs typically expressing transcription factors such as SOX2, NES, PAX6, SOX1, OTX2, OTX1, NKX6.1, FOXA2 or LMX1A, or neural blast cells (NBCs) that typically express transcription factors such as TBR2 or SOX4 or ASCL1, or a mixed population of any of these cell types.

As used herein, the terms “neuron” and “nerve cell” may be used interchangeably referring to neural cells which are post-mitotic have fully developed/terminally differentiated, typically from a pluripotent stem cell into a specialized cell which can transmit nerve impulses. When referring to neural cell as “mitotic” is meant proliferative cells that are in the process of dividing/proliferating or capable of doing so. It follows that when referring to neural cells as “post-mitotic” is meant cells that cannot divide/proliferate. The neural cells according to the present invention may have a specific regional identity, such as cells specific to the forebrain, midbrain, hindbrain, etc. As used herein, the term “forebrain” refers to the rostral region of the neural tube and CNS that gives rise to structures including the cortex and striatum. As used herein, the term “midbrain” refers to the medial region of the neural tube and CNS (on the rostro-caudal axis) that gives rise to structures including the substantia nigra, As used herein, the terms “hindbrain” and “spinal cord” refer to the caudal region of the neural tube that is caudal to the isthmus organiser.

The method according to the present invention is typically defined by a series of method steps. As used herein, the term “step” in relation to the method is to be understood as a stage, where something is undertaking and/or an action is performed. It will be understood by one of ordinary skill in the art when the steps to be performed and/or the steps undertaking are concurrent and/or successive and/or continuous.

Differentiation of Stem Cells into Neural Cells

In the step of culturing PSCs, the cells may be obtained from any suitable source as referred to in the above. By the term “culturing” is meant that the PSCs are cultured in a cell culture medium, which is suitable for viability in their current state of development. Culturing the stem cells typically implies a transfer of the stem cells into a different environment such as by seeding onto a new substrate or suspending in an incubator. One of ordinary skill in the art will readily recognize that stem cells are fragile to such a transfer and the procedure requires diligence and that maintaining the stem cells in the origin cell culture medium may facilitate a more sustainable transfer of the cells before replacing the cell culture medium with another cell culture medium more suitable for the differentiation process.

The step of differentiating the pluripotent stem cells into neural stem precursor blast cells may be by any suitable method for directing the pluripotent stem cells to develop into cells of the ectoderm germ layer. A person skilled in the art will recognize suitable methods available for such differentiation process. Neural stem precursor blast cells can presently be obtained by two major methodologies, firstly via traditional differentiation of pluripotent stem cells into neural stem progenitor cells (NSPCs) via contacting the pluripotent stem cells with additional of exogenous compounds or via direct conversion (also termed transdifferentiation) or forward programming where any cell type is converted to neural stem progenitor cells (NSPCs) via overexpression of identity and master regulator genes or delivery of specific small molecules that switch on identity and master regulator genes, examples of these methods are shown in Zhang et al., Sudhof “Rapid Single-Step Induction of Functional Neurons from Human Pluripotent Stem Cells” 2013, and Vierbuchen et al., Wernig “Direct conversion of fibroblasts to functional neurons by defined factors” 2010.

As used herein, the term “differentiation” refers broadly to the process wherein cells progress from an undifferentiated state or a state different from the intended differentiated state to a specific differentiated state, e.g. from an immature state to a less immature state or from an immature state to a mature state, which may occur continuously as the method is performed. The term “differentiation” in respect to pluripotent stem cells refers to the process wherein cells progress from an undifferentiated state to a specific differentiated state, i.e. from an immature state to a less immature state or to a mature state. Changes in cell interaction and maturation occur as cells lose markers of undifferentiated cells or gain markers of differentiated cells. Loss or gain of a single marker can indicate that a cell has “matured or fully differentiated”. Examples of cell types where efficient two-dimensional protocols are available span the four major domains of the CNS including (a) forebrain cortical glutamatergic neurons (Shi, Kirwan et al. 2012), (b) midbrain dopaminergic neurons (Niclis, Gantner et al. 2017), (c) hindbrain serotonergic neurons (Lu, Zhong et al. 2016), and (d) spinal cord motor neurons (Amoroso, Croft et al. 2013). The time required for the step of differentiating the PSCs into neural stem precursor blast cells depends on the protocol used. A person skilled in the art will be able to determine the progress of the differentiation and what stage the neural cells have developed into. Determining the progress of differentiation may be by analysis of certain expression markers or at certain stages this may be assessed visually.

In an embodiment, the PSCs are differentiated in a two-dimensional culture. In a further embodiment, the PSCs are initially plated on a substrate. In an embodiment, the substrate comprises an extracellular matrix. In a further embodiment, the substrate comprises Poly-L-Lysine, Poly-D-Lysine, Poly-Ornithine, laminin, fibronectin, and/or collagen, and/or fragments thereof. In a more specific embodiment, the laminin or fragment thereof is selected from the group comprising of laminin-111, laminin-521, and laminin-511. In another embodiment, the PSCs are differentiated in a suspension culture.

In an embodiment, at least 10%, 20%, 30%, 40%, 50%, 60%, 70%, 80%, 90%, 95%, or 99% of the cells are neural stem precursor blast cells prior to the step of aggregating the neural stem precursor blast cells to form a neural microsphere.

In an embodiment, the PSCs are differentiated into neural stem precursor blast cells at a stage ranging from early neural progenitor cells to young neurons. Specifically, in an embodiment, the PSCs are differentiated into neural stem precursor blast cells at a stage selected from the group comprising early neural progenitor cells, late neural progenitor cells, neuroblasts, and young neurons. In an embodiment, the PSCs are differentiated for about 3 days to about 40 days into neural stem precursor blast cells, preferably for about 5 days to 30 days. In an embodiment for differentiating the neural stem precursor blast cells into ventral midbrain neural stem precursor blast cells, the pluripotent stem cells are differentiated for about 8 days to about 22 days, more preferably for about 10 days to about 20 days, more preferably for about 12 days to about 18 days, even more preferably for about 14 days to about 17 days, even more preferably for about 16days. In another embodiment for differentiating into cortical neural stem precursor blast cells the cells are differentiated for about 7 days to about 35 days, preferably for 15 days to about 30 days, more preferably for about 20 days to about 30 days, such as for about 25 days to about 28 days. In an embodiment, the neural stem precursor blast cells are differentiated for a period of time so that they express one or more markers selected from the group comprising SOX2, NES, K167, and DCX. Neural stem precursor blast cells of specific regional identities typically express one or more markers. Accordingly, in an embodiment, the neural stem precursor blast cells express one or more markers selected from the group comprising PAX6, OTX2, SOX1, NKX6.1, NKX2.1, LMX1, ISL1, EBF1, OLIG2, FOXA2, EOMES, and PDGFRa.

As the pluripotent stem cells have differentiated into neural stem precursor blast cells they are subjected to a step of aggregation to form a neural microsphere. In a preferred embodiment, at least 10%, 20%, 30%, 40%, 50%, 60%, 70%, 80%, 90%, 95%, or 99% of the neural stem precursor blast cells are along the ectoderm lineage, preferably the neuroectodermal, and not pluripotent and not terminally differentiated at the step of aggregating neural stem precursor blast cells to form a neural microsphere. In an embodiment the PSCs are differentiated for a period of time whereby at least 50%, 60%, 70%, 80%, 90%, 95%, 99%, or 100% of the neural stem precursor blast cells are no longer pluripotent, prior to the step of aggregation. In an embodiment, the PSCs are differentiated for a period of time whereby at least 50%, 60%, 70%, 80%, 90%, 95%, 99%, or 100% of the neural stem precursor blast cells do not express one or more of the markers OCT-3/4, NANOG, SOX2, CD9, SSEA3, SSEA4, TRA160, and TRA180, prior to the step of aggregation. By “prior to the step of aggregation” is meant that the cells develop to a stage thereby having the given properties at the time of aggregation.

### Dissociation Into Single Cell Suspension

In a further embodiment, the method further comprises a step of dissociating the neural stem precursor blast cells to a single cell suspension prior to the step of aggregating the neural stem precursor blast cells. A person skilled in the art will recognize suitable techniques for dissociating the neural stem precursor blast cells in order to ensure viability. In an embodiment, the neural stem precursor blast cells are dissociated enzymatically or by chelating. In an embodiment the neural stem precursor blast cells are dissociated by contacting the neural stem precursor blast cells with a dissociating agent. Non-limiting examples of dissociating agents include accutase, trypsin, trypleSelect, collagenase, disapse, versene, EDTA, and/or ReLeSR. It is well recognized that dissociation of cells may cause stress, and in an embodiment the neural stem precursor blast cells are therefore contacted with a ROCK inhibitor prior to the step of dissociating the neural stem precursor blast cells. In an embodiment, the neural stem precursor blast cells are contacted with a ROCK inhibitor after the step of dissociating the neural stem precursor blast cells, such as for about 12 hours to about 72 hours, preferably for about 24 hours to about 48 hours. The use of a ROCK inhibitor has been shown to suppress dissociation-induced apoptosis. Accordingly, in a particular embodiment a ROCK inhibitor is added during dissociation and/or aggregation steps. In an embodiment the concentration of ROCK inhibitor is from about 0.1 μM to about 30 μM, preferably from about 1 μM to about 10 μM. In an embodiment the ROCK inhibitor is Y-27632. As used herein, “Y-27632” refers to trans-4-(1-Aminoethyl)-N-(4-Pyridyl)-cyclohexane-carboxamide dihydrochloride with CAS no. 129830-38-2.

In an embodiment, the method comprises the additional step of seeding the neural stem precursor blast cells in a well suitable for maintaining a neural microsphere in a static non-adherent culture, prior to the step of aggregating the neural stem precursor blast cells. By the term “seeding” is meant transferring cells such as in single cell suspension to a container suitable for cell aggregation, such as a microwell.

In an embodiment, less than about 1000, 900, 800, 700, 600, 500, 400, 300, 200, or 150 neural stem precursor blast cells are seeded in each microwell, preferably less than about 500 neural stem precursor blast cells per microwell are seeded in the microwell, more preferred less than about 250 neural stem precursor blast cells are seeded in the microwell.

In a further embodiment, more than about 2, 5, 10, 15, 20, 25, 30, 35, 40, 45, 50, 75, or 100 neural stem precursor blast cells are seeded in the microwell, preferably more than about 50 neural stem precursor blast cells are seeded in the microwell, even more preferably more than about 100 neural stem precursor blast cells are seeded in the microwell.

In a further embodiment, from about 10 to about 1000 neural stem precursor blast cells, preferably from about 50 to about 500 neural stem precursor blast cells, more preferably from about 50 to about 250 neural stem precursor blast cells, even more preferably from about 100 to about 250 neural stem precursor blast cells, are seeded in the microwell.

### Cell Aggregation and Microsphere Formation

The step of aggregating the neural stem precursor blast cells to form neural microspheres may be carried out passively or actively as long as the cells form clusters. Accordingly, in an embodiment, the neural stem precursor blast cells are aggregated by gravitational settling of the neural stem precursor blast cells in the single cell suspension. In another embodiment, the neural stem precursor blast cells are aggregated by spin-aggregation of the neural stem precursor blast cells in the single cell suspension. In such an embodiment, the spin-aggregation forms the neural stem precursor blast cells into a neural microsphere. In an embodiment, the spin-aggregation is by centrifugation. In a more specific embodiment, the centrifugal force is from about 5 gs to about 800 gs, preferably from about 100 gs to about 400 gs. In an embodiment, the neural stem precursor blast cells are added as a cell suspension and left under stationary conditions, thereby allowing the cells in suspensions to settle by the force of gravity into microwells, which subsequently form aggregates.

Throughout the method according to the present invention, the cells may be maintained in any suitable cell culture vessel, which supports either a two-dimensional or three-dimensional culturing of the cells depending on the differentiation protocol applied. As used herein, the term “cell culture vessel” is defined as a container specifically designed to support the growth and propagation of cells in culture. The container may vary in size, shape, coating, and the presence or absence of a lid. Non-limiting examples of cell culture vessels include cell culture dishes, tubes, wells, and flasks.

For the step of aggregating the neural stem precursor blast cells the cell culture vessel must accommodate the formation of the neural microsphere and the cells may be transferred to such. In an embodiment, the neural stem precursor blast cells are seeded in a cell culture vessel accommodating the formation of neural microspheres subsequent to dissociation. In an embodiment, the method comprises the additional step of transferring the neural stem precursor blast cells to a cell culture well suitable for maintaining a neural microsphere prior to the step of aggregating the neural stem precursor blast cells. In a further embodiment, the cell culture well is suitable for maintaining the neural microsphere in a static non-adherent culture. As used herein, by the term “well” is meant a space suitable for a single microsphere to be maintained in a static non-adherent culture. By the term “static non-adherent culture” is meant that cells are placed in a condition where they are not adhered to a surface and suspended in a media solution but one which does not employ motion, with forces of gravity being the only means to hold cells in position. Cell culture vessels may comprise a well, which functions as a parent housing, wherein one or more additional smaller wells, optionally referred to as microwells, are comprised. Accordingly, in an embodiment, the cell culture well is a microwell.

As used herein, the term “microwell” refers to wells as described in WO2008106771. Throughout the present application the cell culture vessel may be referred to as a “microwell”. This is not to be construed as limiting. Embodiments as presented herein which are referring to the microwell may equally apply to another suitable cell culture vessel mutatis mutandis. In an embodiment, the cell culture well suitable for maintaining the neural microsphere in a static non-adherent culture has a surface with low cell attachment properties. By “low cell attachment properties” is meant that a material has a low affinity for adhesion with cells and prevents their direct binding and growth on said material. Further to this, in an embodiment the surface with low cell attachment properties is low-adherent plastic and/or plastic treated with a low-adherent agent. Microwells with the aforementioned properties are readily available. Suitable microwells are described in EP2230014 and WO2008106771.

In an embodiment, multiple neural microspheres are obtained simultaneously, such as by initially differentiating and proliferating neural stem precursor blast cells in suspension culture and transferring to a multiwell allowing for the formation of multiple neural microspheres in individual microwells.

In an embodiment, the method comprises the additional steps of collecting the neural stem precursor blast cells and transferring the neural stem precursor blast cells to a well suitable for maintaining a neural microsphere in a static non-adherent culture, prior to the step of aggregating the neural stem precursor blast cells.

In a preferred embodiment, the cell culture vessel such as the microwell features low-cell attachment properties. Such properties are typical for non-treated plastics that have not been exposed to plasma gasses, which would typically add oxygen-containing functional groups such as hydroxyl and carboxyl to modify the hydrophobic plastic surface, thereby making the surface more hydrophilic. The present inventors believe that the low-attachment properties of the cell culture vessel facilitate the maturation process as it prevents microsphere attachment and neurite growth to the plastic, which would otherwise allow cells to migrate. In a preferred embodiment, only one neural microsphere is cultured in each well.

Furthermore, in an embodiment, the cell culture vessel is not coated with an extracellular matrix component. It follows that in a preferred embodiment, the surface of the cell culture vessel is free of an extracellular matrix.

In an embodiment, the step of aggregating the neural stem precursor blast cells to form neural microspheres comprises aggregating from about 5 to about 1000 neural stem precursor blast cells. In an embodiment, less than about 1000, 900, 800, 700, 600, 500, 400, 300, 250, or 150 neural stem precursor blast cells are aggregated, preferably less than about 500 neural stem precursor blast cells are aggregated. In an embodiment, more than about 5, 10, 15, 20, 25, 30, 35, 40, 45, 50, 75, or 100 neural stem precursor blast cells are aggregated, preferably more than about 50 neural stem precursor blast cells are aggregated, even more preferably more than about 100 neural stem precursor blast cells are aggregated. In a further embodiment, from about 10 to about 1000 neural stem precursor blast cells, preferably from about 50 to about 500 neural stem precursor blast cells, more preferably from about 100 to about 500 neural stem precursor blast cells, are aggregated forming a neural microsphere.

### Maturation of the Neural Microspheres

In a preferred embodiment the neural stem precursor blast cells of the neural microsphere are allowed to further mature prior to an optional step of collecting the neural microsphere. As used herein, by the term “maturing” is meant a further development of stem cells that have already undergone an initial differentiation towards a specific germ layer. The terms “maturation” and “allowing to mature” may also be considered a further differentiation of the cells. Maturation will typically be the further development of a progenitor or precursor cell towards a definitive cell type. Likely depending on the cell types maturation may simply require maintenance of the cells, such as by replacing the cell culture medium and ensuring viable conditions. Maturation of the cells may also require exposing the cells to further compounds or factors in order to facilitate the further development towards the definitive cell type. As used herein, the cells development of the cells from a pluripotent stage to a definitive cell type is referred to as “differentiation” prior to the formation of the microsphere and to as “maturation” following the formation of the microsphere. Depending on the timing of the formation of the microsphere the development of the cells towards the definitive cell type may occur before or after the formation of the microsphere.

In a preferred embodiment, the neural stem precursor blast cells are further matured in a static non-adherent culture. Accordingly, the neural microsphere is maintained in a static non-adherent culture for the further maturation of the neural stem precursor blast cells.

In an embodiment, the neural stem precursor blast cells are allowed to further mature into neurons. In an embodiment, the neural stem precursor blast cells of the neural microsphere are allowed to further mature for at least about 2.5 days to about 200 days, preferably for about 3 days to about 180 days, more preferably for about 5 days to about 150 days, more preferably for about 10 days to about 120 days, more preferably for about 10 days to about 100 days, more preferably for about 10 days to about 60 days, more preferably for about 15 days to about 50 days, more preferably for about 15 days to about 40 days. In an embodiment, the neural stem precursor blast cells of the neural microsphere are allowed to further mature for no longer than about 200, 190, 180, 170, 160, 150, 140, 130, 120, 110, 100 days. In an embodiment, the neural stem precursor blast cells of the neural microsphere are allowed to further mature for at least about 2.5, 3, 5, 8, 10, or 15 days, preferably for at least about 15 days. A person skilled in the art will acknowledge that different neural cell types require different time for maturing, especially depending on the protocol applied. In a particular embodiment for obtaining ventral midbrain neural stem precursor blast cells, the cells are allowed to further mature for about 10 days to about 30 days into neurons, preferably for about 16 days to about 19 days. In another embodiment for obtaining cortical neural stem precursor blast cells, the cells are allowed to further mature for about 25 days to about 40 days into neurons, preferably for about 25 days to about 35 days, more preferably for about 32 days. In an embodiment, the neurons express one or more markers selected from the group comprising DCX, NEUN, INA, Beta Tubulins, Microtubule Associated Proteins, TH, GABA, vGLUTs, and ChAT. In an embodiment, the neurons do not express one or more of the markers selected from the group comprising SOX2, Ki67, and NES.

Generation of NSPBCs via differentiation methods can produce highly homogenous populations of these cells, and with specific regional identities.

Examples of differentiation to ventral midbrain NSPBCs is found in the publications by Nolbrant et al., Kirkeby “ Generation of high-purity human ventral midbrain dopaminergic progenitors for in vitro maturation and intracerebral transplantation” 2017, Kriks et al., Studer “Dopamine neurons derived from human ES cells efficiently engraft in animal models of Parkinson's disease” 2011 and Doi et al., Takahashi “Isolation of Human Induced Pluripotent Stem Cell-Derived Dopaminergic Progenitors by Cell Sorting for Successful Transplantation” 2014.

Examples of differentiation to dorsal forebrain/pallial NSPBCs is found in the publications by Shi et al., Livesey “Human cerebral cortex development from pluripotent stem cells to functional excitatory synapses” 2011 and Espuny-Camacho et al., Vanderhaegen

“Pyramidal Neurons Derived from Human Pluripotent Stem Cells Integrate Efficiently into Mouse Brain Circuits In Vivo” 2012.

Examples of differentiation to hindbrain/spinal cord NSPBCs is found in the publications by Du et al., Zhang “Generation and expansion of highly pure motor neuron progenitors from human pluripotent stem cells” 2014; Amaroso et al., Wichterle “Accelerated High-Yield Generation of Limb-Innervating Motor Neurons from Human Stem Cells” 2013; Butts et al., McDevitt “V2a interneuron differentiation from mouse and human pluripotent stem cells” 2019.

In an embodiment, the inhibitor of Small Mothers Against Decapentaplegic (SMAD) protein signaling comprises more than one compound, such as but not limited to a combination of the aforementioned inhibitors of Small Mothers Against Decapentaplegic (SMAD) protein signaling. A person skilled in the art will recognize that the concentration of the individual inhibitors of Small Mothers Against Decapentaplegic (SMAD) protein signaling may need to be adjusted accordingly to obtain similar effect as one would with the individual inhibitors. In an embodiment for ventral midbrain dopamine neural stem precursor cell specification, the PSCs exposed to SMAD inhibitor(s) are contacted with an inhibitor of Small Mothers Against Decapentaplegic (SMAD) protein signaling pathway.

In an embodiment, the PSCs are contacted with one or more of the compounds selected from the group consisting of Sonic Hedgehog (SHH) agonist such as SHH or SAG, CHIR99021, FGF8, FGF2, retinoic acid, BDNF, GDNF, dcAMP, DAPT, and ascorbic acid (AA).

In an embodiment, PSCs are contacted with a Sonic Hedgehog (SHH) agonist, such as SHH or SAG. In an embodiment, PSCs are contacted with a WNT activator or GSK3 inhibitor, such as CHIR99021. In an embodiment, PSCs are contacted with fibroblast growth factor (FGF) 8.

In an embodiment for forebrain pallial/cortical neural stem precursor blast cell specification, the PSCs are contacted with an inhibitor of (SMAD) protein signaling pathway for about 0-10 days and then contacted with FGF2 for about 7-9 days and cultured in basal neural media for a further 9-12 days.

### Filtering, Collection and Cryopreservation

In an embodiment, the neural microsphere is collected following the step of aggregation. Specifically, the neural microsphere is collected after the step of allowing the neural stem precursor blast cells of the neural microsphere to further mature. By “collecting” is meant that the neural microsphere is moved or recovered to a container for suitable for storage or subsequent use. This could be for cryopreservation or administration.

In an embodiment, the method comprises the additional step of cryopreserving the collected neural microsphere. Several cryoprotectants are commercially available and any suitable cryoprotectant may be used.

In one embodiment, the method further comprises the additional step of transferring the obtained neural microsphere to an in vitro two-dimensional culture. In a further embodiment, the method further comprises the additional step of allowing neurite outgrowth of the neural microsphere.

In an embodiment, the method further comprises the additional step of transplanting the obtained neural microsphere to a patient. In another embodiment, the method comprises the additional step of transplanting the obtained neural microsphere to an animal, such as a rodent.

In an embodiment, the method further comprises the additional step of filtering the single cell suspension prior to aggregation. The addition of a filtration step prior to e.g. centrifugation of a cell suspension ensures only single cells or aggregates of a desired dimension are further matured. In an embodiment, the method further comprises an additional step of filtering the neural microspheres. This step is carried out after formation of the neural microsphere to remove any sub-optimal neural microsphere that is larger than the desired size.

In an embodiment of the present invention, the method for obtaining neural microspheres comprises the steps of culturing PSCs, differentiating the PSCs into neural stem precursor blast cells, optionally, filtering the neural stem precursor blast cells, aggregating the neural stem precursor blast cells to form a neural microsphere, allowing the neural stem precursor blast cells of the neural microsphere to further mature, optionally, filtering the neural microsphere, collecting the neural microsphere, and optionally, cryopreserving the neural microsphere.

### Characteristics of the Neural Microsphere

Another aspect of the present invention relates to a neural microsphere. Specifically, a neural microsphere obtainable according to the methods as described herein. More specifically, a neural microsphere obtained according to any of the methods as described herein. In an embodiment, the neural microsphere is in vitro. By the term “in vitro” is meant that the neural microsphere is provided and maintained outside of the human or animal body. In an embodiment, wherein the neural cells are non-native. By the term “non-native” is meant that the neural microsphere although derived from pluripotent stem cells, which may have human origin, is an artificial construct, that does not exist in nature. In general, it is an object within the field of stem cell therapy to provide cells, which resemble the cells of the human body as much as possible. However, it may never become possible to mimic the development which the pluripotent stem cells undergo during the embryonic and fetal stage to such an extent that the mature cells are indistinguishable from native cells of the human body. Inherently, in an embodiment of the present invention, the neural cells of the neural microsphere are artificial. As used herein, the term “artificial” may comprise material naturally occurring in nature but modified to a construct not naturally occurring. This includes human stem cells, which are differentiated into non-naturally occurring cells mimicking the cells of the human body. Preferably, the neural cells of the neural microsphere are stem cell-derived. More preferably, the neural cells are stem cell-derived from pluripotent stem cells. In a further embodiment, the neural cells are stem cell-derived from human embryonic stem cells (hESCs) and/or human induced pluripotent stem cells (hiPSCs).

In the following, the characteristics of the neural microsphere are described in more details. The neural microsphere as such is an artificial construct forming a non-native structure of stem cell-derived neural cells. In a general embodiment of the present invention, the neural microsphere comprises cells of the ectoderm lineage. In a further embodiment, the neural microsphere comprises post-mitotic cells of the ectoderm lineage. In a more specific embodiment, the ectoderm lineage is the neuroectoderm lineage.

Without being bound by any particular theory it is believed that the neural cells have a natural affinity to one another and form a tight mesh-like structure, which in a static environment with low adherence allow the neural cells to form the sphere-like geometry.

In an embodiment, the diameter of the neural microsphere is less than about 300, 250, 200, 150, 100, 90, 80, 70, 60, 50, 40, or 30 μm, preferably less than 250 μm. In an embodiment, the diameter of the neural microsphere is larger than about 1, 5, 10, 15, 20, 25, 30, 35, 40, or 45 μm, preferably larger than 10 μm. Additionally, in an embodiment, the diameter of the neural microsphere is from about 10 μm to about 300 μm, preferably from about 10 μm to about 250 μm, preferably from about 10 μm to about 150 μm, more preferably from about 10 pm to about 100 μm, more preferably from about 10 μm to about 55 μm, more preferably from about 10 μm to about 50 μm, more preferably from about 20 μm to about 50 μm, more preferably from about 30 μm to about 50 μm. A person skilled in the art will acknowledge that the neural microsphere does not form a perfect sphere. However, the nature of the microsphere obtainable according to the aforementioned methods naturally forms a sphere-like structure for which a diameter can readily be established using a microscope. In an embodiment, the microsphere is spherical in shape. A person skilled in the art will recognize that a microsphere composed of living cells will not form an ideal sphere shape, but the aggregate of cells will be spherical in appearance. Accordingly, the microsphere may be considered substantially spherical in shape. As that the neural microsphere substantially forms a sphere, the diameter and volume may be calculated by determining the polar axis as an average of the major and minor axes, which essentially minimizes the irregularity of each aggregate by assuming the roundest shape possible.

With the aforementioned methods the present inventors aim at providing a neural microsphere, wherein the entire volume of microsphere consists of neural cells. A person skilled in the art will recognize that the microsphere according to the present invention comprises living cells and that this inherently introduces variability into the product. In particular, the cells may vary in size by growing or shrinking during different stages, the number of cells in the microsphere may not be constant due to proliferation or apoptosis. Dead cells may be broken down and cell membranes destroyed, thus rupturing the cell and releasing cellular contents. The cells may also secrete cellular material during different stages of development. Accordingly, it is to be understood that a microsphere comprising neural cells or consisting of neural cells may also comprise cellular material originating from the neural cells.

Therefore, in an embodiment, is provided a neural microsphere, wherein the entire volume of microsphere consists of neural cells and optionally cellular material originating from the neural cells. As used herein, the term “cellular material originating from the neural cells” means secreted proteins or other molecules and includes cellular debris originating from dead cells. Due to the aforementioned nature of the microsphere comprising living cells the microsphere product may also be defined as a neural microsphere, wherein the entire volume of microsphere substantially consists of neural cells. Further to this, an embodiment relates to a neural microsphere, wherein the entire volume of microsphere substantially consists of neural cells and optionally cellular material originating from the neural cells. In an embodiment, at least 50%, 60%, 70%, 80%, 90%, 95%, or 100% of the volume of the neural microsphere comprises neural cells, preferably at least 90%, more preferably at least 95%. In an embodiment, at least 50%, 60%, 70%, 80%, 90%, 95%, or 100% of the volume of the neural microsphere comprises neural cells and optionally cellular material originating from the neural cells, preferably at least 90% of the volume of the neural microsphere comprises neural cells and optionally cellular material originating from the neural cells, more preferably at least 95% of the volume of the neural microsphere comprises neural cells and optionally cellular material originating from the neural cells. In a preferred embodiment, the volume of the neural microsphere substantially consists of neural cells. In an embodiment, at least 50%, 60%, 70%, 80%, 90%, 95%, or 100% of the neural microsphere consists of neural cells, preferably at least 90%, more preferably at least 95%. It follows that in an embodiment, the volume of the neural microsphere substantially consists of neural cells and optionally cellular material originating from the neural cells. In an embodiment, at least 50%, 60%, 70%, 80%, 90%, 95%, or 100% of the neural microsphere consists of neural cells and optionally cellular material originating from the neural cells, preferably at least 90% of the neural microsphere consists of neural cells and optionally cellular material originating from the neural cells, more preferably at least 95% of the neural microsphere consists of neural cells and optionally cellular material originating from the neural cells.

In an embodiment, the distribution of the neural cells throughout the neural microsphere is homogenous. In an embodiment, the distribution of the neural cells throughout the neural microsphere is even. Again, a person skilled in the art will recognize the inherent variability of a product comprising living cells. Accordingly, the distribution of the neural cells throughout the neural microsphere may also be referred to as substantially homogenous, or in an embodiment, the distribution of the neural cells throughout the neural microsphere may be referred to as substantially even, wherein the term “substantially” when used in connection with cellular material, such as when referring to distribution of cells in a microsphere, is to be understood with a certain degree of variability inherent to the product. In a preferred embodiment, the neural microsphere does not comprise a lumen. By the term “lumen” is meant a hollow space inside the neural microsphere, which is not occupied by neural cells and optionally cellular material originating from the neural cells. Such lumen could be aqueous, containing a liquid such as a culture medium. As used herein, the term “lumen” is defined as a hollow space having the size of three cells or more.

In an embodiment, the neural microsphere is at least 50%, 60%, 70%, 80%, 90%, 95%, or 99% saturated with neural cells. In another embodiment, the neural microsphere is at least 50%, 60%, 70%, 80%, 90%, 95%, or 99% saturated with neural cells and optionally cellular material originating from the neural cells.

In an embodiment, the neural microsphere comprises from about 5 to about 1000 neural cells, preferably from about 30 to about 500 neural cells, more preferably from about 50 to about 250 neural cells. In an embodiment, the neural microsphere comprises less than about 1000, 900, 800, 700, 600, 500, 400, 300, 200, 150, or 100 neural cells, preferably less than about 500 neural cells, more preferably less than about 250 neural cells. In an embodiment, more than about 5, 10, 15, 20, 25, 30, 35, 40, 45, or 50 neural cells, preferably more than about 50 neural cells.

In an embodiment, at least 50%, 60%, 70%, 80%, 90%, 95% or 100% of the neural cells are viable, preferably at least 50% of the neural cells are viable, preferably at least 70% of the neural cells are viable, preferably at least 90% of the neural cells are viable. As used herein, by the term “viable” in reference to cells is meant that the cells have not undergone and/or are not undergoing cell death, such as apoptosis. A person skilled in the art will recognize that death of cells occurs as a normal and controlled part of growth or development. Accordingly, part of the neural cells of the neural microsphere may be undergoing cell death. Characteristic of the microsphere of the present invention is the smaller size, which prevents substantial necrosis of cells at the core of the sphere, which is typically pronounced in spheres having a larger diameter, such as about 1,000 microns.

In a preferred embodiment, the neural microsphere comprises less than about 50%, 40%, 30%, 20%, 10%, 9%, 8%, 7%, 6%, 5%, 4%, 3%, 2%, or 1% of cells other than neural cells, preferably the neural microsphere comprises less than about 10% of cells other than neural cells, more preferably less than 5% of cells other than neural cells, even more preferably less than 1% of cells other than neural cells. In an even more preferred embodiment, the neural microsphere is devoid of cells other than neural cells. Accordingly, in a preferred embodiment, the neural microsphere consists of neural cells.

In an embodiment, at least 10% of the neural cells are neurons. In an embodiment, the neural cells are neurons, wherein at least 60%, 70%, 80%, 90%, or 95% of the neural cells express the neuronal marker NEUN, preferably at least 90%, more preferably at least 95%.

In an embodiment, at least 50%, 60%, 70%, 80%, 90%, 95%, 100% of the neural cells are post-mitotic neural lineage cells, preferably at least 70% of the neural lineage cells are post-mitotic neural lineage cells, more preferably 80% of the neural lineage cells are post-mitotic, even more preferably 90% of the neural lineage cells are post-mitotic. In an embodiment, the post-mitotic neural lineage cells are neurons.

Typically, according the present invention a neural microsphere comprising a majority of mitotic neural cells will be an intermediate product. The present inventors anticipate that the therapeutic applicability of neural microspheres primarily comprising post-mitotic neural lineage cells is preferred over products having cells which are capable of dividing and are further from becoming terminally matured cells. In an embodiment, the neurons express markers selected from the group comprising of DCX, NEUN, INA, Beta Tubulins, Microtubule Associated Proteins, TH, GABA, vGLUTs, ChAT. Specifically, in an embodiment, the neural cells express TH. In an embodiment, the neurons do not express one or more markers selected from the group comprising SOX2, Ki67 or NES. In an embodiment, the neurons are hindbrain-spinal cord neurons. In another embodiment, the neurons are forebrain neurons. In another embodiment, the neurons are midbrain neurons.

In an embodiment, the neural cells express NEUN and do not express SOX2. In an embodiment, wherein the neural cells co-express FOXA2 and NEUN. In an embodiment, at least 50%, 60%, 70%, 80%, 90%, or 95% of the neural cells co-express the markers FOXA2 and LMX1A, preferably at least 80%, more preferably at least 90%. In a further embodiment, at least 50% of the neural cells co-expressing the markers FOXA2 and LMX1A further co-express the marker PITX3. Such cells are considered suitable for the treatment of Parkinson's disease. In one embodiment, the neural cells are dopaminergic progenitor cells. In an embodiment, the neural cells co-express BRN2 and TBR1. In a preferred embodiment, the neural microsphere is substantially free of exogenous extracellular matrix. In a more preferred embodiment, the neural microsphere is free of exogenous extracellular matrix. As used herein, the term “exogenous” means any matter that has been added to the neural microsphere, i.e. not produced by the cells themselves. Neural cells may naturally produce e.g. extracellular matrix, which may then form part of the neural microsphere. However, in a preferred embodiment, the neural cells of the neural microsphere are not brought into contact with exogenous extracellular matrix and exogenous extracellular matrix does not form part of the neural microsphere. Similarly, in a preferred embodiment, the neural microsphere is substantially free of exogenous hydrogel. In a more preferred embodiment, the neural microsphere is free of exogenous hydrogel. As used herein, the term “hydrogel” refers to natural polymers, which may include proteins such as collagen and gelatin and polysaccharides such as starch, alginate, and agarose. Accordingly, in an embodiment, the hydrogel is selected from the group consisting of collagen, gelatin, starch, alginate, and agarose. In an embodiment, the neural microsphere is substantially free of exogenous alginate. In a preferred embodiment, the neural microsphere is free of exogenous alginate.

In an embodiment, the neural microsphere is not enclosed. In an embodiment, the neural cells are not enclosed. As used herein, by the term “enclosed” is meant that the neural cells and/or neural microsphere are not encapsulated by a layer of exogenous material, such as an extracellular matrix or hydrogel. In a preferred embodiment, the neural cells are directly exposed at the surface of the neural microsphere. By “directly exposed” is meant that the most outer layer of the neural cells of the neural microsphere may come in direct contact with anything that comes in the vicinity of the neural microsphere. This is to be understood as different from an enclosed neural microsphere, where an outer encapsulation would prevent a direct contact with other cells or large molecules that would come in the vicinity of the neural microsphere. Accordingly, in an embodiment the surface of the neural microsphere comprises neural cells. In an embodiment, the surface of the neural microsphere comprises at least about 10%, 20%, 30%, 40%, 50%, 60%, 70%, 80%, 90%, 95%, or 99% neural cells, preferably at least about 50% neural cells, more preferably at least about 80% neural cells. Accordingly, in an embodiment the neural microsphere comprises an outer layer of neural cells. In an embodiment, the outer layer comprises at least about 10%, 20%, 30%, 40%, 50%, 60%, 70%, 80%, 90%, 95%, or 100% neural cells, preferably at least about 50% neural cells, more preferably at least about 80% neural cells. As used herein, the terms “outer layer” and “surface” in reference to the neural microsphere may be used interchangeably and refer to the neural cells forming the surface of the neural microsphere, i.e. the neural cells that are not fully surrounded by other neural cells but are partly exposed to the surroundings. In a preferred embodiment, the outer layer of the neural microsphere consists of neural cells.

In an embodiment, less than 80%, 90%, or 95%, or none of the neural cells have formed neurites extending more than one neural cell diameter radially from the neural microsphere. This may be determined by microscopically observing either the static microspheres inside the microwell or upon removal and transfer to another container. As used herein, the term “neurite” means any projection from the body of a nerve cell (neuron), such as but not limited to an axon or a dendrite. By the term “extending radially” is meant that the neurite is projected outwards from the surface of the neural microsphere, e.g. extending into a surrounding medium. It is believed that the neural cells of the neural microsphere form neurites in a mesh inside the neural microsphere. Without being bound by any particular theory it is believed that maintaining the neural microsphere in a static condition in a cell culture vessel with low adherent surface will limit the formation of neurites extending radially from the neural microsphere. This also restrains neurites within the microsphere and adhesion to the surface does not occur, allowing for the easy transport and movement of the microsphere to a new condition, i.e. re-plating in vitro or delivery through surgical devices to the CNS. The present inventors have shown, however, that the neural cells are still capable of neurite growth and extension. In an embodiment of the invention, at least 50% of the neurons form neurites extending radially from the neural microsphere within around 120 minutes after seeding in vitro in a two-dimensional culture system made to support neurite attachment and growth; see FIGS. 3 and 4.

Neural microspheres can be tested for their neurite attachment and growth properties by transferring microspheres to plastic plates or flasks that are coated with human or mouse laminin, Matrigel or other like extracellular matrices that support neurite attachment and growth. Microspheres must be cultured in appropriate media to support cell viability and growth of neural fibers for this procedure.

### Composition and Administration

Another aspect of the present invention relates to a composition comprising neural microspheres.

In an embodiment, the composition is for the use as a medicament. In a further embodiment, the composition is for the treatment of a neural condition selected from the group comprising of Parkinson's disease, stroke, traumatic brain injury, spinal cord injury, Huntington's disease, dementia, Alzheimer's disease, and other neurological conditions wherein neurons are lost or dysfunctional. In one particular embodiment, the composition comprises neural microspheres, wherein the neural cells of the neural microsphere are neurons, specifically dopaminergic cells, for the treatment of Parkinson's disease. In one embodiment, the composition to be administered for the treatment of the neurological condition comprises from about 1000 microspheres to about 100,000 microspheres per treatment per patient. In an embodiment for the treatment of Parkinson's disease the composition comprises from about 1000 microspheres to about 50,000 microspheres, wherein each microsphere comprises from about 50 cells to about 500 cells.

In an embodiment of the composition, the average diameter of the neural microspheres is less than 300, 250, 200, 150, 130, 100, 80, 70, 65, 60, or 50 μm, preferably less than 250 μm. In an embodiment of the composition, the diameter of the neural microspheres is less than 300, 250, 200, 150, 130, 100, 80, 70, 65, 60, or 50 μm, preferably less than 250 μm. In an embodiment, the average diameter of the neural microspheres is between 50 μm to 250 μm.

Another aspect relates to a container comprising a composition according to the previous aspect of the present invention.

In another aspect, the present invention provides a cryopreserved composition comprising neural microspheres according to any of the aforementioned embodiments.

Another aspect relates to a method for the treatment of a neurological condition comprising the administration of a neural microsphere or composition thereof. In a particular embodiment the method is for the treatment of Parkinson's disease, stroke, traumatic brain injury, spinal cord injury, Huntington's disease, dementia, Alzheimer's disease, and other neurological conditions wherein neurons are lost or dysfunctional. In an embodiment, the treatment of a neurological condition comprises the administration of a neural microsphere. In a further embodiment, the administration is by transplantation of the neural microsphere into the CNS. In an even further embodiment, the administration into the CNS is carried out using a delivery device comprising means for injecting the neural microsphere. In an embodiment, the means for injecting the neural microsphere is a needle, wherein the diameter of the needle is from about 0.1 mm to about 2 mm, preferably from about 0.5 mm to about 1 mm.

Another aspect relates to a method for pretreatment of a needle for administration of a neural microsphere, wherein the needle is pre-coated with an anti-adherence solution. An example of an anti-adherence solution is Anti-adherence Rinsing Solution provided by STEMCELL Technologies (https://www.stemcell.com/aggrewell-rinsing-solution.html#section-overview). In an embodiment, the needle is filled with an anti-adherence solution. In a further embodiment, the needle is washed with HBSS without Ca²⁺ and Mg²⁺ after being emptied for the anti-adherence solution.

### Generic Application of the Microsphere Protocol

The methods for providing stem cell-based microspheres according to the present invention are also applicable to cells of the other germ layers. The present inventors have successfully obtained stem cell-based microspheres comprising cardiomyocytes and pancreatic islet-like cells, respectively.

Accordingly, an aspect of the present invention relates to the generic application of the methods described herein, specifically a method for obtaining a stem cell-based microsphere, comprising the steps of differentiating PSCs to obtain differentiated cells, aggregating the differentiated cells to form a stem cell-based microsphere comprising cells, and allowing the cells of the stem cell-based microsphere to further mature.

By the term “stem cell-based microsphere” is to be understood a microsphere as previously defined, comprising cells which have been derived from stem cells. By the term “differentiated cells” is meant cells, which have undergone or are undergoing a process wherein the cells progress from an undifferentiated state to a specific differentiated state, i.e.

from an immature state to a less immature state. Typically, “differentiated cells” have not matured fully into their terminal fate, thus, allowing the differentiated cells to undergo a further step of maturation in order to further mature into such terminal fate. The cells may be differentiated into any type of cells, including the three different germ layers: endoderm, ectoderm and mesoderm. Definitions and embodiments as described hereinbefore may equally apply to this generalizing aspect mutatis mutandis. This includes aforementioned details on single cell suspension, aggregation, etc. In an embodiment, the method comprises the further step of dissociating the differentiated cells into single cell suspension, prior to the step of aggregation. If follows, in an embodiment the differentiated cells are aggregated by spin-aggregation. In a preferred embodiment, the cells are maintained in a static non-adherent culture prior to the step of aggregation. In a preferred embodiment, the cells are maintained in a well comprising a surface with low cell attachment properties prior to the step of aggregation.

In an embodiment, from 10 to 1000 differentiated cells are aggregated to form the cell-based microsphere. In an embodiment, the cells of the cell-based microsphere are of the ectoderm lineage, and the diameter of the cell-based microsphere is from 50 to 250 micrometers. In an embodiment, the cells of the cell-based microsphere are of the endoderm lineage, and the diameter of the cell-based microsphere is from 30 to 350 micrometers.

In an embodiment of the method, the PSCs are differentiated for a period of time whereby at least 50%, 60%, 70%, 80%, 90%, 95%, 99%, or 100% of the differentiated cells are no longer pluripotent, prior to the step of aggregation. In an embodiment, the PSCs are differentiated for a period of time whereby at least 50%, 60%, 70%, 80%, 90%, 95%, 99%, or 100% of the differentiated cells do not express one or more of the markers OCT-3/4, NANOG, SOX2, CD9, SSEA3, SSEA4, TRA160, and TRA180, prior to the step of aggregation. In an embodiment, the PSCs are differentiated for at least 2, 3, 4, 5, 6, 7, 8, 9, or 10 days prior to aggregating the differentiated cells, preferably at least 2 days, more preferably at least 5 days, and even more preferably at least 10 days.

### Microspheres Comprising Neural Cells

In a particular embodiment, the cells of the stem cell-based microsphere are ectodermal cells, and the PSCs are differentiated for 10 to 35 days prior to aggregating the differentiated cells. The stem cell-based microsphere comprising ectodermal cells include the neural microsphere as described hereinbefore. In a further embodiment, the cells of the stem cell-based microsphere are dorsal forebrain pallial neural cells, and wherein the PSCs are differentiated for 20 to 35 days prior to aggregating the differentiated cells. In another embodiment, the cells of the stem cell-based microsphere are ventral midbrain cells, and wherein the PSCs are differentiated for 10 to 25 days prior to aggregating the differentiated cells. In another embodiment, the cells of the stem cell-based microsphere are ventral hindbrain and/or spinal cord cells, and wherein the PSCs are differentiated for 5 to 25 days prior to aggregating the differentiated cells.

In an embodiment, the cells of the cell-based microsphere are ectodermal, and wherein from 10 to 1000 differentiated cells are aggregated to form the cell-based microsphere, preferably from 100 to 500 differentiated cells, more preferably from 100 to 250 differentiated cells.

One intended application of the neural microspheres is for the treatment of Parkinson's disease. It is widely recognized that such treatment requires the administration of dopaminergic cells into the region of the patient's brain referred to as the striatum for heterotopic transplantation or substantia nigra for homotopic transplantation. The neural cells of that region is characterized by the co-expression of the markers FOXA2, LMX1A, and PITX3. Accordingly, for the treatment of Parkinson's disease a microsphere with such cells is anticipated. Therefore, an embodiment of the present invention relates to neural microsphere, wherein at least 50%, 60%, 70%, 80%, 90%, or 95% of the neural cells co-express the markers FOXA2 and LMX1A, preferably at least 80%, more preferably at least 90%. In a further embodiment, at least 50% of the neural cells co-expressing the markers FOXA2 and LMX1A further co-express the marker PITX3.

A particular embodiment relates to a neural microsphere for the treatment of Parkinson's disease, wherein the neural microsphere comprises from about 100 to about 500 neural cells, and wherein at least 80% of the neural cells co-express FOXA2, LMX1A, and TH, and wherein the diameter of the neural microsphere ranges from about 50 to about 250 μm, and wherein at least 90% of the volume of the neural microsphere consists of neural cells, and wherein the distribution of the neural cells inside the neural microsphere is even and wherein the neural microsphere is free of exogenous extracellular matrix and free of exogenous hydrogel.

### Microspheres Comprising Cardiomyocytes

In a particular embodiment, the cells of the stem cell-based microsphere are mesodermal cells, and wherein the PSCs are differentiated for 5 to 10 days prior to aggregating the differentiated cells. In a further embodiment, the cells of the stem cell-based microsphere are cardiomyocytes, and wherein the PSCs are differentiated for 5 to 10 days prior to aggregating the differentiated cells. By “cardiomyocytes” is to be understood cells of the mesodermal lineage that are a major component of the heart organ, wherein the cells express markers such as NKX2.5.

In an embodiment, the cells of the cell-based microsphere are mesodermal, and wherein from 50 to 3000 differentiated cells are aggregated to form the cell-based microsphere, preferably from 500 to 1500 differentiated cells are aggregated.

In an embodiment, the cell-based microsphere is less than 350 μm in diameter.

### Microspheres Comprising Pancreatic Islet-Like Cells

In a particular embodiment, the cells of the stem cell-based microsphere are endodermal cells, and wherein the PSCs are differentiated for 10 to 20 days prior to aggregating the differentiated cells. In a further embodiment, the cells of the stem cell-based microsphere are pancreatic islet-like cells, and wherein the PSCs are differentiated for 10 to 20 days prior to aggregating the differentiated cells. By “pancreatic islet-like cells” is to be understood cells of the endodermal lineage that are a component of pancreatic islets, typically expressing markers such as C-Peptide, NKX6.1 and Glucagon.

In an embodiment, the cells of the cell-based microsphere are endodermal, and wherein 50 to 3000 differentiated cells are aggregated to form the cell-based microsphere, preferably from 500 to 1500 differentiated cells are aggregated.

In an embodiment, the cell-based microsphere is less than 350 μm in diameter.

### Particular Embodiments

The aspects of the present invention are now further described by the following non-limiting embodiments:
1. A method for obtaining a neural microsphere, comprising the steps of:

providing neural stem precursor blast cells,

aggregating the neural stem precursor blast cells to form a neural microsphere, and

allowing the neural stem precursor blast cells of the neural microsphere to further mature.
2. The method according to the preceding embodiment, wherein the neural microsphere is collected after the step of allowing the neural stem precursor blast cells of the neural microsphere to further mature.
3. The method according to any one of the preceding embodiment, wherein providing the neural stem precursor blast cells comprises the step of:

differentiating PSCs into neural stem precursor blast cells.
4. The method according to any one of the preceding embodiments, wherein providing the neural stem precursor blast cells comprises the initial step of culturing PSCs.
5. The method according to any one of the preceding embodiments, wherein the neural stem precursor blast cells are allowed to further mature into neurons.
6. The method according to any one of the preceding embodiments, wherein the PSCs are differentiated for about 3 days to about 40 days into neural stem precursor blast cells, preferably for about 5 days to about 30 days.
7. The method according to any one of the preceding embodiments, wherein the PSCs are differentiated into ventral midbrain neural stem precursor blast cells for about 8 days to about 25 days, preferably for about 10 days to about 20 days, more preferably for about 12 days to about 18 days, more preferably for about 14 days to about 17 days, even more preferably for about 16 days.
8. The method according to any one of the preceding embodiments, wherein the PSCs are differentiated into cortical neural stem precursor blast cells for about 20 days to about 35 days, preferably for about 25 days to about 30 days, more preferably for about 28 days.
9. The method according to any one of the preceding embodiments, wherein the PSCs are differentiated for a period of time whereby at least 50%, 60%, 70%, 80%, 90%, 95%, 99%, or 100% of the neural stem precursor blast cells are no longer pluripotent, prior to the step of aggregation.
10. The method according to any one of the preceding embodiments, wherein the PSCs are differentiated for a period of time whereby at least 50%, 60%, 70%, 80%, 90%, 95%,99%, or 100% of the neural stem precursor blast cells do not express one or more of the markers OCT-3/4, NANOG, SOX2, CD9, SSEA3, SSEA4, TRA160, and TRA180, prior to the step of aggregation.
11. The method according to any one of the preceding embodiments, wherein the neural stem precursor blast cells express one or more markers selected from the group comprising SOX2, NES, KI67, ASCL1, TBR2, DCX PAX6, OTX2, SOX1, NKX6.1, NKX2.1, ISL1, EBF1, OLIG2, LMX1, FOXA2, EOMES, and PDGFRa.
12. The method according to any one of the preceding embodiments, wherein at least 10%, 20%, 30%, 40%, 50%, 60%, 70%, 80%, 90%, 95%, or 99% of the neural stem precursor blast cells are along the ectoderm lineage, preferably the neuroectodermal, and not pluripotent and not terminally differentiated at the step of aggregating neural stem precursor blast cells to form a neural microsphere.
13. The method according to any one of the preceding embodiments, wherein the neural stem precursor blast cells of the neural microsphere are allowed to further mature for about 2.5 day to about 200 days, preferably for about 3 days to about 180 days, more preferably for about 5 days to about 150 days, more preferably for about 10 days to about 120 days, more preferably for about 10 days to about 100 days, more preferably for about 10 days to about 60 days, more preferably for about 15 days to about 50 days, more preferably for about 15 days to about 40 days.
14. The method according to any one of the preceding embodiments, wherein the neural stem precursor blast cells of the neural microsphere are allowed to further mature for no longer than about 200, 190, 180, 170, 160, 150, 140, 130, 120, 110, 100 days.
15. The method according to any one of the preceding embodiments, wherein the neural stem precursor blast cells of the neural microsphere are allowed to further mature for at least about 2.5, 3, 5, 8, 10, or 15 5days, preferably for at least about 15 days.
16. The method according to any one of embodiments 1 to 7 and 11 to 15, wherein the neural stem precursor blast cells are differentiated into ventral midbrain neural stem precursor blast cells and allowed to further mature for about 10 days to about 30 days into neurons, preferably for about 16 days to about 19 days.
17. The method according to any one of embodiments 1 to 6 and 8 to 15, wherein the neural stem precursor blast cells are differentiated into cortical neural stem precursor blast cells and allowed to further mature for about 25 days to about 40 days into neurons, preferably for about 25 days to about 35 days, more preferably for about 32 days.
18. The method according to any one of the preceding embodiments, wherein the neurons express one or more markers selected from the group comprising DCX, NEUN, INA, Beta Tubulins, Microtubule Associated Proteins, TH, GABA, vGLUTs, and ChAT.
19. The method according to any one of the preceding embodiments, wherein the neurons do not express one or more markers selected from the group comprising SOX2, Ki67, and NES.
20. The method according to any one of the preceding embodiments, wherein the PSCs are differentiated in a two-dimensional culture.
21. The method according to any one of the preceding embodiments, wherein the PSCs are initially plated on a substrate.
22. The method according to embodiment 21, wherein the substrate comprises an extracellular matrix.
23. The method according to embodiment 22, wherein the substrate comprises Poly-L-Lysine, Poly-D-Lysine, Poly-Ornithine, laminin, fibronectin, and/or collagen, and/or fragments thereof.
24. The method according to embodiment 23, wherein the laminin or fragment thereof is selected from the group comprising of laminin-111, laminin-521, and laminin-511.
25. The method according to any one of the preceding embodiments, wherein the PSCs are differentiated in a suspension culture.
26. The method according to any one of the preceding embodiments, comprising the step of dissociating the neural stem precursor blast cells to a single cell suspension prior to the step of aggregating the neural stem precursor blast cells.
27. The method according to embodiment 26, wherein the neural stem precursor blast cells are dissociated enzymatically or by chelating.
28. The method according to embodiment 27, wherein the neural stem precursor blast cells are dissociated by contacting the neural stem precursor blast cells with a dissociating agent.
29. The method according to embodiment 28, wherein the dissociating agent is selected from the group comprising accutase, trypsin, trypleSelect, collagenase, disapse versene, EDTA, and ReLeSR.
30. The method according to any one of embodiments 26 to 29, wherein the neural stem precursor blast cells are contacted with a ROCK inhibitor after the step of dissociating the neural stem precursor blast cells, such as for about 12 hours to about 72 hours, preferably for about 24 hours to about 48 hours.
31. The method according to any one of embodiments 26 to 30, wherein the neural stem precursor blast cells are contacted with a ROCK inhibitor prior to the step of dissociating the neural stem precursor blast cells.
32. The method according to any one of embodiments 30 and 31, wherein the ROCK inhibitor is Y-27632.
33. The method according to any one of the preceding embodiments, comprising the additional step of filtering the single cell suspension prior to aggregation.
34. The method according to any one of the preceding embodiments, wherein the neural stem precursor blast cells are further matured in a static non-adherent culture.
35. The method according to any one of the preceding embodiments, comprising the additional step of seeding the neural stem precursor blast cells in a well suitable for maintaining a neural microsphere in a static non-adherent culture, prior to the step of aggregating the neural stem precursor blast cells.
36. The method according to embodiment 35, wherein less than about 1000, 900, 800, 700, 600, 500, 400, 300, 200, or 150 neural stem precursor blast cells are seeded in the well, preferably less than about 500 neural stem precursor blast cells per microwell are seeded in the well, even more preferred less than about 250 neural stem precursor blast cells are seeded in the well.
37. The method according to any one of embodiments 35 and 36, wherein more than about 2, 5, 10, 15, 20, 25, 30, 35, 40, 45, 50, 75, or 100 neural stem precursor blast cells are seeded in the well, preferably more than about 50 neural stem precursor blast cells are seeded in the well, even more preferably more than about 100 neural stem precursor blast cells are seeded in the well.
38. The method according to any one of embodiments 35 to 37, wherein from about 10 to about 1000 neural stem precursor blast cells, preferably from about 50 to about 500 neural stem precursor blast cells, more preferably from about 50 to about 250 neural stem precursor blast cells, even more preferably from about 100 to about 250 neural stem precursor blast cells, are seeded in the well.
39. The method according to any one of the embodiments 35 to 38, wherein the well suitable for maintaining the neural microsphere in a static non-adherent culture is a microwell.
40. The method according to any one of embodiments 35 to 39, wherein the well suitable for maintaining the neural microsphere in a static non-adherent culture has a surface with low cell attachment properties.
41. The method according to embodiment 40, wherein the surface with low cell attachment properties is low-adherent plastic and/or plastic treated with a low-adherent agent.
42. The method according to any one of embodiments 35 to 41, wherein the surface of the well is free of an extracellular matrix.
43. The method according to any one of the preceding embodiments, wherein the neural stem precursor blast cells are aggregated by gravitational settling of the neural stem precursor blast cells in the single cell suspension.
44. The method according to any one of the preceding embodiments, wherein the neural stem precursor blast cells are aggregated by spin-aggregation of the neural stem precursor blast cells in the single cell suspension.
45. The method according to embodiment 44, wherein the spin-aggregation forms the neural stem precursor blast cells into a neural microsphere.
46. The method according to any one of embodiments 44 and 45, wherein the spin-aggregation is by centrifugation.
47. The method according to any one of embodiments 44 to 46, wherein the centrifugal force is from about 5 gs to about 800 gs, preferably from about 100 gs to about 400 gs.
48. The method according to any one of the preceding embodiments, wherein from about 10 to about 1000 neural stem precursor blast cells are aggregated.
49. The method according to any one of the preceding embodiments, wherein less than about 1000, 900, 800, 700, 600, 500, 400, 300, 250, or 150 neural stem precursor blast cells are aggregated, preferably less than about 500 neural stem precursor blast cells are aggregated.
50. The method according to any one of the preceding embodiments, wherein more than about 5, 10, 15, 20, 25, 30, 35, 40, 45, 50, 75, or 100 neural stem precursor blast cells are aggregated, preferably more than about 50 neural stem precursor blast cells are aggregated, even more preferably more than about 100 neural stem precursor blast cells are aggregated.
51. The method according to any one of the preceding embodiments, wherein from about 10 to about 1000 neural stem precursor blast cells, preferably from about 50 to about 500 neural stem precursor blast cells, more preferably from about 100 to about 500 neural stem precursor blast cells, are aggregated.
52. The method according to any one of the preceding embodiments, wherein the neural microsphere is maintained in a static non-adherent culture for the further maturation of the neural stem precursor blast cells.
53. The method according to any one of the preceding embodiments, wherein the PSCs are contacted with an inhibitor of Small Mothers Against Decapentaplegic (SMAD) protein signaling.
54. The method according to any one of the preceding embodiments, wherein PSCs are contacted with a Sonic Hedgehog (SHH) agonist, such as SHH or SAG.
55. The method according to any one of the preceding embodiments, wherein PSCs are contacted with a WNT activator and/or GSK3 inhibitor, such as CHIR99021.
56. The method according to any one of the preceding embodiments, wherein PSCs are contacted with fibroblast growth factor (FGF) 8.
57. The method according to any one of the preceding embodiments, wherein PSCs are contacted with ascorbic acid.
58. The method according to any one of the preceding embodiments, wherein PSCs are contacted with BDNF.
59. The method according to any one of the preceding embodiments, wherein neural stem precursor blast cells are further matured by contacting the cells with GDNF.
60. The method according to any one of the preceding embodiments, wherein neural stem precursor blast cells are further matured by contacting the cells with dcAMP.
61. The method according to any one of the preceding embodiments, wherein neural stem precursor blast cells are further matured by contacting the cells with DAPT.
62. The method according to any one of the preceding embodiments, wherein neural stem precursor blast cells are further matured by contacting the cells with FGF2.
63. The method according to any one of the preceding embodiments, wherein neural stem precursor blast cells are further matured by contacting the cells with retinoic acid.
64. The method according to any one of the preceding embodiments comprising the additional step of cryopreserving the neural microsphere.
65. The method according to any one of the preceding embodiments, comprising the additional step of transferring the obtained neural microsphere to an in vitro two-dimensional culture.
66. The method according to embodiment 65, comprising the additional step of allowing neurite outgrowth of the neural microsphere transferred to the in vitro two-dimensional culture.
67. The method according to any one of embodiments 1 to 64, comprising the additional step of transplanting the obtained neural microsphere to a patient.
68. A method for obtaining a neural microsphere, comprising the steps of:

culturing PSCs,

differentiating the PSCs into neural stem precursor blast cells,

optionally, filtering the neural stem precursor blast cells,

aggregating the neural stem precursor blast cells to form a neural microsphere,

allowing the neural stem precursor blast cells of the neural microsphere to further mature,

optionally, filtering the neural microsphere,

collecting the neural microsphere, and

optionally, cryopreserving the neural microsphere.
69. A neural microsphere comprising neural cells.
70. The neural microsphere according to embodiment 69, which neural microsphere is obtainable according to method of any of embodiments 1 to 64, or 68.
71. The neural microsphere according to embodiment 69, which neural microsphere is obtained according to method of any of embodiments 1 to 64, or 68.
72. The neural microsphere according to any one of embodiments 69 to 71, wherein the neural microsphere is in vitro.
73. The neural microsphere according to any one of embodiments 69 to 72, wherein the neural cells are non-native.
74. The neural microsphere according to any one of embodiments 69to 73, wherein the neural cells are artificial.
75. The neural microsphere according to any one of embodiments 69 to 74, wherein the neural cells are stem cell-derived.
76. The neural microsphere according to any one of embodiments 69 to 75, wherein the neural cells are stem cell-derived from pluripotent stem cells.
77. The neural microsphere according to any one of embodiments 69 to 76, wherein the neural cells are stem cell-derived from human embryonic stem cells (hESCs) or human induced pluripotent stem cells (hiPSCs).
78. The neural microsphere according to any one of embodiments 69 to 77, wherein the diameter of the neural microsphere is less than about 300, 250, 200, 150, 100, 90, 80, 70, 60, 50, 40, or 30 μm, preferably less than about 250 μm.
79. The neural microsphere according to any one of embodiments 69 to 78, wherein the diameter of the neural microsphere is larger than about 1, 5, 10, 15, 20, 25 μm, preferably larger than 10 μm.
80. The neural microsphere according to any one of embodiments 69 to 79, wherein the diameter of the neural microsphere is from about 10 μm to about 300 μm, preferably from about 10 μm to about 250 μm, preferably from about 10 μm to about 150 μm, more preferably from about 10 μm to about 100 μm, more preferably from about 10 μm to about 55 μm, more preferably from about 10 μm to about 50 μm, more preferably from about 20 μm to about 50 μm, more preferably from about 30 μm to about 50 μm.
81. The neural microsphere according to any one of embodiments 69 to 80, wherein the neural microsphere is substantially spherical in shape.
82. The neural microsphere according to any one of embodiments 69 to 81, wherein the neural microsphere is at least 50%, 60%, 70%, 80%, 90%, 95%, or 99% saturated with neural cells.
83. The neural microsphere according to any one of embodiments 69 to 81, wherein the neural microsphere is at least 50%, 60%, 70%, 80%, 90%, 95%, or 99% saturated with neural cells and optionally cellular material originating from the neural cells.
84. The neural microsphere according to any one of embodiments 69 to 83, wherein the neural microsphere comprises from about 5 to about 1000 neural cells, preferably from about 30 to about 500 neural cells, more preferably from about 50 to about 250 neural cells.
85. The neural microsphere according to any one of embodiments 69 to 84, wherein the neural microsphere comprises less than about 1000, 900, 800, 700, 600, 500, 400, 300, 200, 150, or 100 neural cells, preferably less than about 500 neural cells, more preferably less than about 250 neural cells.
86. The neural microsphere according to any one of embodiments 69 to 85, wherein the neural microsphere comprises more than about 5, 10, 15, 20, 25, 30, 35, 40, 45, or 50 neural cells, preferably more than about 50 neural cells.
87. The neural microsphere according to any one of embodiments 69 to 86, wherein at least 50%, 60%, 70%, 80%, 90%, 95% or 100% of the neural cells are viable, preferably at least 50% of the neural cells are viable, preferably at least 70% of the neural cells are viable, preferably at least 90% of the neural cells are viable.
88. The neural microsphere according to any one of embodiments 69 to 87, wherein the surface of the neural microsphere comprises neural cells.
89. The neural microsphere according to embodiment 88, wherein the surface of the neural microsphere comprises at least about 10%, 20%, 30%, 40%, 50%, 60%, 70%, 80%, 90%, 95%, or 99% neural cells, preferably at least about 50% neural cells, more preferably at least about 80% neural cells.
90. The neural microsphere according to any one of embodiments 69 to 89, wherein the surface of the neural microsphere consists of neural cells.
91. The neural microsphere according to any one of embodiments 69 to 90, wherein the neural cells are directly exposed at the surface of the neural microsphere.
92. The neural microsphere according to any one of embodiments 69 to 91, wherein the neural microsphere comprises an outer layer of neural cells.
93. The neural microsphere according to any one of embodiments 69 to 92, wherein the outer layer of the neural microsphere comprises neural cells.
94. The neural microsphere according to any one of embodiments 92 and 93, wherein the outer layer comprises at least about 10%, 20%, 30%, 40%, 50%, 60%, 70%, 80%, 90%, 95%, or 99% neural cells, preferably at least about 50% neural cells, more preferably at least about 80% neural cells.
95. The neural microsphere according to any one of embodiments 69 to 94, wherein the outer layer of the neural microsphere consists neural cells.
96. The neural microsphere according to any one of embodiments 69 to 95, wherein the neural cells are not enclosed.
97. The neural microsphere according to any one of embodiments 69 to 96, wherein the neural microsphere is not enclosed.
98. The neural microsphere according to any one of embodiments 69 to 97, wherein at least 50%, 60%, 70%, 80%, 90%, 95%, or 100% of the volume of the neural microsphere comprises neural cells, preferably at least 90% of the volume of the neural microsphere comprises neural cells, more preferably at least 95% of the volume of the neural microsphere comprises neural cells.
99. The neural microsphere according to any one of embodiments 69 to 97, wherein at least 50%, 60%, 70%, 80%, 90%, 95%, or 100% of the volume of the neural microsphere comprises neural cells and optionally cellular material originating from the neural cells, preferably at least 90% of the volume of the neural microsphere comprises neural cells and optionally cellular material originating from the neural cells, more preferably at least 95% of the volume of the neural microsphere comprises neural cells and optionally cellular material originating from the neural cells.
100. The neural microsphere according to any one of embodiments 69 to 99, wherein at least 50%, 60%, 70%, 80%, 90%, 95%, or 100% of the neural microsphere consists of neural cells, preferably at least 90% of the neural microsphere consists of neural cells, more preferably at least 95% of the neural microsphere consists of neural cells.
101. The neural microsphere according to any one of embodiments 69 to 99, wherein at least 50%, 60%, 70%, 80%, 90%, 95%, or 100% of the neural microsphere consists of neural cells and optionally cellular material originating from the neural cells, preferably at least 90% of the neural microsphere consists of neural cells and optionally cellular material originating from the neural cells, more preferably at least 95% of the neural microsphere consists of neural cells and optionally cellular material originating from the neural cells.
102. The neural microsphere according to any one of embodiments 69 to 101, wherein the distribution of the neural cells throughout the neural microsphere is substantially homogenous.
103. The neural microsphere according to any one of embodiments 69 to 102, wherein the volume of the neural microsphere consists of neural cells.
104. The neural microsphere according to any one of embodiments 69 to 102, wherein the volume of the neural microsphere consists of neural cells and optionally cellular material originating from the neural cells.
105. The neural microsphere according to any one of embodiments 69 to 104, wherein the distribution of the neural cells inside the neural microsphere is even.
106. The neural microsphere according to any one of embodiments 69 to 105, wherein the neural microsphere does not comprise a lumen.
107. The neural microsphere according to any one of embodiments 69 to 106, wherein the neural microsphere is free of exogenous extracellular matrix.
108. The neural microsphere according to any one of embodiments 69 to 107, wherein the microsphere is free of exogenous hydrogel.
109. The neural microsphere according to any one of embodiments 69 to 108, wherein the neural microsphere comprises less than about 50%, 40%, 30%, 20%, 10%, 9%, 8%, 7%, 6%, 5%, 4%, 3%, 2%, or 1% of cells other than neural cells, preferably the neural microsphere comprises less than about 10% of cells other than neural cells.
110. The neural microsphere according to any one of embodiments 69 to 109, wherein the neural microsphere consists of neural cells.
111. The neural microsphere according to any one of embodiments 69 to 110, wherein the neural cells express NEUN and do not express SOX2.
112. The neural microsphere according to any one of embodiments 69 to 111, wherein the neural cells co-express FOXA2 and NEUN.
113. The neural microsphere according to any one of embodiments 69 to 112, wherein the neural cells express TH.
114. The neural microsphere according to any one of embodiments 69 to 113, wherein the neural cells express BRN2 or TBR1.
115. The neural microsphere according to any one of the embodiments 69 to 114, wherein at least 20%, 30%, 40% 50%, 60%, 70%, 80%, 90%, or 95% of the neural cells co-express the markers FOXA2 and LMX1A, preferably at least 80%, more preferably at least 90%.
116. The neural microsphere according to embodiment 115, wherein at least 20% of the neural cells co-expressing the markers FOXA2 and LMX1A further co-express the marker PITX3.
117. The neural microsphere according to embodiment 69 to 116, wherein the at least 10% of the neural cells are neurons.
118. The neural microsphere according to embodiment 69 to 117, wherein the neural cells are neurons, wherein at least 60%, 70%, 80%, 90% or 95% of the neural cells expresses the neuronal marker NEUN, preferably at least 90% and more preferably at least 95%.
119. The neural microsphere according to any one of embodiments 117 and 118, wherein the neurons are hindbrain-spinal cord neurons.
120. The neural microsphere according to any one of embodiments 117 to 119, wherein the neurons are forebrain neurons.
121. The neural microsphere according to any one of embodiments 117 to 119, wherein the neurons are midbrain neurons.
122. The neural microsphere according to any one of embodiments 117 to 121, wherein the neurons express markers selected from the group consisting of DCX, NEUN, INA, Beta Tubulins, Microtubule Associated Proteins, TH, GABA, vGLUTs, and ChAT.
123. The neural microsphere according to any one of embodiments 69 to 122, wherein the neurons do not express one or more of the markers selected from the group comprising SOX2, Ki67, and NES.
124. The neural microsphere according to any one of embodiments 69 to 123, wherein at least 50%, 60%, 70%, 80%, 90%, 95%, 100% of the neural cells are post-mitotic neural lineage cells, preferably at least 70% of the neural cells are post-mitotic neural lineage cells, preferably 80% of the neural cells are post-mitotic, preferably 90% of the neural lineage cells are post-mitotic.
125. The neural microsphere according to any one of embodiments 69 to 124, wherein the neural cells are dopaminergic progenitor cells.
126. The neural microsphere according to any one of embodiments 69 to 125, wherein the neural cells express the markers selected from the group consisting of DCX, NEUN, INA, Beta Tubulins, Microtubule Associated Proteins, TH, GABA, vGLUTs, and ChAT.
127. The neural microsphere according to any one of embodiments 69 to 126, wherein less than 80%, 90%, or 95%, or none of the neural cells have formed neurites extending more than one neural cell diameter radially from the neural microsphere.
128. The neural microsphere according to any one of embodiments 69 to 127, wherein at least 50% of the neurons form neurites extending radially from the neural microsphere within around 120 minutes after seeding in vitro in a two-dimensional culture.
129. A neural microsphere according to any one of embodiments 69 to 128 for use as a medicament.
130. A neural microsphere according to any one of embodiments 69 to 128 for the treatment of Parkinson's disease, stroke, traumatic brain injury, spinal cord injury, Huntington's disease, dementia, Alzheimer's disease, and other neurological conditions wherein neurons are lost or dysfunctional.
131. A method for the treatment of a neurological condition comprising the administration of a neural microsphere according to any one of the embodiments 69 to 128.
132. The method according to embodiment 131, for the treatment of Parkinson's disease, stroke, traumatic brain injury, spinal cord injury, Huntington's disease, dementia, Alzheimer's disease, and other neurological conditions wherein neurons are lost or dysfunctional.
133. The method according to any one of embodiments 131 and 132, wherein the administration is by transplantation of the neural microsphere into the CNS.
134. The method according to embodiment 133, wherein the administration into the CNS is carried out using a delivery device comprising means for injecting the neural microsphere.
135. The method according to embodiment 134, wherein the means for injecting the neural microsphere is a needle, wherein the diameter of the needle is from about 0.1 mm to about 2 mm.
136. A composition comprising neural microspheres according to any one of embodiments 69 to 128.
137. The composition according to embodiment 136, wherein the average diameter of the neural microspheres is less than 300, 250, 200, 150, 130, 100, 80, 70, 65, 60, or 50 μm, preferably less than 250 μm.
138. The composition according to any one of embodiments 136 and 137, wherein the diameter of the neural microspheres is less than 300, 250, 200, 150, 130, 100, 80, 70, 65, 60, or 50 μm, preferably less than 250 μm.
139. The composition according to any one of the embodiments 136 to 138, wherein the average diameter of the neural microspheres is between 50 μm to 250 μm.
140. A composition comprising neural microspheres according to any one of embodiments 69 to 128 for use as a medicament.
141. A composition comprising neural microspheres according to any one of embodiments 69 to 128 for the treatment of Parkinson's disease, stroke, traumatic brain injury, spinal cord injury, Huntington's disease, dementia, Alzheimer's disease, and other neurological conditions wherein neurons are lost or dysfunctional.
142. A container comprising a composition according to any one of embodiments 136 to 141.
143. A cryopreserved composition comprising neural microspheres according to any one of embodiments 136 to 141.
144. A method for obtaining a stem cell-based microsphere, comprising the steps of:

differentiating PSCs to obtain differentiated cells,

aggregating the differentiated cells to form a stem cell-based microsphere, and

allowing the differentiated cells of the stem cell-based microsphere to further mature.
145. The method according to embodiment 144, wherein the PSCs are differentiated for a period of time whereby at least 50%, 60%, 70%, 80%, 90%, 95%, 99%, or 100% of the differentiated cells are no longer pluripotent, prior to the step of aggregation.
146. The method according to any one of the embodiments 144 to 145, wherein the PSCs are differentiated for a period of time whereby at least 50%, 60%, 70%, 80%, 90%, 95%, 99%, or 100% of the differentiated cells are not pluripotent and do not express one or more of the markers OCT-3/4, NANOG, CD9, SSEA3C, SSEA4, TRA160, and TRA180, prior to the step of aggregation.
147. The method according the any one of embodiments 144 to 146, comprising the further step of dissociating the differentiated cells into single cell suspension, prior to the step of aggregation.
148. The method according to any one of embodiments 144 to 147, wherein the PSCs are differentiated for at least 2, 3, 4, 5, 6, 7, 8, 9, or 10 days prior to aggregating the differentiated cells, preferably at least 2 days, more preferably at least 5 days, and even more preferably at least 10 days.
149. The method according to embodiment 148, wherein the cells of the stem cell-based microsphere are ectodermal cells, and wherein the PSCs are differentiated for 10 to 35 days prior to aggregating the differentiated cells.
150. The method according to embodiment 149, wherein the cells of the stem cell-based microsphere are dorsal forebrain pallial neural cells, and wherein the PSCs are differentiated for 20 to 35 days prior to aggregating the differentiated cells.
151. The method according to embodiment 149, wherein the cells of the stem cell-based microsphere are ventral midbrain cells, and wherein the PSCs are differentiated for 10 to 25 days prior to aggregating the differentiated cells.
152. The method according to embodiment 149, wherein the cells of the stem cell-based microsphere are ventral hindbrain and/or spinal cord cells, and wherein the PSCs are differentiated for 5 to 25 days prior to aggregating the differentiated cells.
153. The method according to embodiment 148, wherein the cells of the stem cell-based microsphere are mesodermal cells, and wherein the PSCs are differentiated for 5 to 10 days prior to aggregating the differentiated cells.
154. The method according to embodiment 153, wherein the cells of the stem cell-based microsphere are cardiomyocytes, and wherein the PSCs are differentiated for 5 to 10 days prior to aggregating the differentiated cells.
155. The method according to embodiment 148, wherein the cells of the stem cell-based microsphere are endodermal cells, and wherein the PSCs are differentiated for 10 to 20 days prior to aggregating the differentiated cells.
156. The method according to embodiment 155, wherein the cells of the stem cell-based microsphere are pancreatic islet-like cells, and wherein the PSCs are differentiated for 10 to 20 days prior to aggregating the differentiated cells.
157. The method according to any one of embodiments 144 to 156, wherein from 10 to 1000 differentiated cells are aggregated to form the cell-based microsphere.
158. The method according to any one of embodiments 149 to 152, wherein the cells of the cell-based microsphere are ectodermal, and wherein from 50 to 1000 differentiated cells are aggregated to form the cell-based microsphere, preferably from 100 to 500 differentiated cells, more preferably from 100 to 250 differentiated cells.
159. The method according to any one of embodiments 153 to 154, wherein the cells of the cell-based microsphere are mesodermal, and wherein from 50 to 3000 differentiated cells are aggregated to form the cell-based microsphere, preferably from 500 to 1500 differentiated cells are aggregated.
160. The method according to any one of embodiments 155 to 156, wherein the cells of the cell-based microsphere are endodermal, and wherein from 50 to 3000 differentiated cells are aggregated to form the cell-based microsphere, preferably from 500 to 1500 differentiated cells are aggregated.
161. The method according to any one of embodiments 144 to 160, wherein the cells of the cell-based microsphere are of the ectoderm or endoderm lineage, and the diameter of the cell-based microsphere is from 30 to 350 micrometers.
162. The method according to any one of embodiments 144 to 161, wherein the differentiated cells are aggregated by spin-aggregation.
163. The method according to any one of embodiments 144 to 162, wherein the cells are maintained in a static non-adherent culture prior to the step of aggregation.
164. The method according to any one of embodiments 144 to 163, wherein the cells are maintained in a well comprising a surface with low cell attachment properties prior to the step of aggregation.

### EXAMPLES

The following are non-limiting examples for carrying out the invention.

### Example 1

### Neural Microsphere Methodologies

First described in brief is the formation of neural microspheres comprised of pre-differentiated but not terminally differentiated cells (i.e. neural stem cells) without extracellular matrices or other additives and their maturation to a terminal cell fate (i.e. neurons), and is as follows:
1. Cultures of NSPBCs obtained by differentiation of hPSCs in either a 2D or 3D format are disassociated to a single cell suspension using an enzyme, chelator or similar molecule. Cell suspensions can also be obtained by thawing cryopreserved NSPBCs.
2. NSPBC suspensions are transferred to plasticware that is lined with microwells at a concentration and total number of cells sufficient so that, after sedimentation by passive gravitational forces or centrifugal forces, each microwell comprises a total number of cells in the range of 5-1000 cells, with numbers adjusted to account for cell death incurred.
3. NSPBC suspension and plate are preferably centrifuged at a speed sufficient to ensure centrifugal forces cause cells to collect in the center-bottom of microwells, typically 50-400 g.
4. Culture of microsphere plates in static non-adherent conditions to allow cellular suspension to accrete into a single cluster or microsphere.
5. Cell concentrates within microwells should, per microwell, comprise a small enough number of cells so that the accreted cluster of live cells are of approximate diameter of ≤250 Ξm as this is feasible for loading into central nervous system (CNS) delivery devices and transplantation to the brain.
6. Terminal maturation of microsphere cells by culture in static non-adherent conditions for extended duration (2 to 200 days) until the majority of mitotic NSPBCs have matured to terminally differentiated cell types such as post-mitotic neurons
7. Transfer of terminally-matured microspheres to in vitro 2D cultures for attachment and neurite outgrowth or transplantation in vivo (i.e to the CNS) for engraftment
8. Pre-coating of transplantation device and cell culture plasticware with an anti-adherence solution preventing microspheres from adhering to glass, plastic or other surfaces.

Secondly described is in extended and greater detail the formation of neural microspheres without extracellular matrices or other additives, along with the methodologies for supporting assays and techniques used to generate data contained within this invention. This is as follows:

Maintenance of Human Pluripotent Stem Cells: Human Embryonic stem cell (hESCs) lines RC17 (Roslin) and 3053 (Novo Nordisk line) were cultured in iPS Brew media (Miltenyi) supplemented with penicillin and streptomycin on human laminin-521 matrix (0.7-1.2 μg/cm²; Biolamina) coated culture ware. The RC17 cell line were kept for <32 passages, and the 3053 cell line was used <30 passages from stocks of 12 and 14 passages, respectively. Media was changed daily, and cells passaged with EDTA 0.5 mM (Thermo Fisher) every 4-6 days. Cultures were maintained at 37° C., humidity 95% and a 5% CO₂ level. All cell cultures were confirmed mycoplasma negative by routine testing.

Ventral midbrain dopaminergic neuron differentiation: Cells were differentiated according to an established protocol (Nolbrant et al., 2017; Kirkeby et al., 2016). In brief, hESC were grown to 70-90% confluency, then disassociated with 0.5 mM EDTA to generate small aggregates of approximately 2-10 cells. The cells were seeded at 1×10⁴ cells/cm² in cell culture flasks or plates coated with human laminin-111 (1.2 pg/cm²; BioLamina) and immediately put into contact with differentiation media. Cells were exposed to N2-based media from days in vitro (DIV) 0-8; 50% DMEM/F12+Glutamax(Gibco) 50% Neurobasal (Gibco), 1% N2 supplement CTS (Thermo Fisher), 5% GlutaMAX (Thermo Fisher), 0.2% Penicillin streptomycin (P/S; Thermo Fisher) and supplemented with SMAD inhibitors SB431542 (10 μM; Miltenyi), Noggin (100 ng/mL; Miltenyi) for neural induction, Sonic Hedgehog C24ll (SHH; 500 ng/mL; Miltenyi) for ventral fate, GSK3β inhibitor CHIR99021 (CHIR; 0.5 μM for 3053 and 0.6 μM RC17; Miltenyi) to promote caudalisation. N2-based media was supplemented with fibroblast growth factor 8b (FGF8b; 100 ng/mL; Miltenyi) from DIV9-11. At DIV11 cells were dissociated with accutase (Innovative Cell Technologies) and seeded at 0.8×10⁶ cells/m² in a cell culture flask or plate coated with human laminin-111 (10 1.2 pg/cm²) in DIV11-16 media (Neurobasal, 2% B27 supplement without vitamin A CTS (Thermo Fisher), 5% GlutaMAX, 0.2% P/S and supplemented with FGF8b (100 ng/mL), L-ascorbic acid (AA; 200 μM; Sigma), human Brain Derived Neurotrophic Factor (BDNF; 20 ng/mL; Miltenyi)) supplemented with rock inhibitor Y27632 (Tocris) at 10 μM. At DIV16 the cells were dissociated with accutase (Innovative Cell Technologies) and either used for microsphere formation, cryopreserved, or re-seeded at 0.155×10⁶ cells/cm² in cell culture flasks/plates coated with human laminin-111 (1.2 μg/cm²) in DIV16 media with 10 μM ROCK inhibitor Y27632 (Tocris) for extended adherent culture allowing the NSPBCs to mature into neurons. From DIV16 onwards, the cells were cultured in a B27-based media supplemented with AA (200 pM), BDNF (20 ng/mL), human Glial Derived Neurotrophic Factor (GDNF; 20 ng/mL; R&D Systems), Dibuturyl-cAMP (cAMP; 500 pM; Sigma) and notch inhibitor DAPT (10 pM; R&D Systems).

Dorsal forebrain cortical glutamatergic neuron differentiation: Cells were differentiated according to an established protocol (Shi et al. 2011; Shi et al 2012). hESCs were seeded and cultured on laminin-521 (1.2 μg/cm²) , BioLamina) coated culture ware, and once forming a 95-100% confluent monolayer exposed to differentiation media. From DIV0-10, the cells were cultured in an N2/B27-based media: 50% DMEM/F12+Glutamax (Gibco) 50% Neurobasal (Gibco), 2% B27 supplement with vitamin A CTS (Thermo Fisher), 1% N2 supplement CTS (Thermo Fisher), 5% GlutaMAX (Thermo Fisher), 0.2% Penicillin streptomycin (P/S; Thermo Fisher), 1% NEAA (Gibco), 0.089% 11-Mercaptoethanol (Gibco), supplemented with SMAD inhibitors SB431542 (10 μM; Miltenyi) and LDN-193189 (100 nM, Tocris). On DIV10, the cells were dissociated with 0.5 mM EDTA and passaged at a 1:2 ratio. N2/B27-based media was supplemented with fibroblast growth factor b (20ng/mL; R&D) from DIV11-18. On DIV18, the cells were dissociated with 0.5 mM EDTA and either cryopreserved or passaged at a 1:2 ratio. From DIV19 onwards, the cells were cultured in N2/B27-based media. Microspheres were generated at DIV27 and 34.

Neural microsphere formation and static non-adherent culture and differentiation: AggreWell™ 24-well plates (Stem Cell Technologies), in which each well contains 1200 microwells of 400 μm×400 μm, were used for microsphere formation and static non-adherent culture. First, the AggreWells were pre-treated with an anti-adherence solution (Stem Cell Technologies) according to the manufacturer's instructions in order to prevent cell adhesion to the plastic. Microspheres were generated by transferring 1 mL of a suitable concentration of NSPBC single cell (or small clusters of 2-10 cells) suspension to the AggreWells depending the desired size of the microspheres (e.g. 1.2×10\hat{ }6 cells/well for generating microspheres consisting of 100 cells). The cells were then settled into the microwells by centrifugal forces by centrifugation at 200-400 g for 5 minutes or were allowed to sediment passively with normal gravitational forces. The plates were kept in standard cell culture incubators at 37° C. with a 5% CO₂ and 95% humidity. Within 24-48 hours after seeding, the cells spontaneously aggregated into uniform spherical microspheres. Media changes were performed by manually removing half of the media and adding fresh media every 2-3 days. The microspheres were maintained in static non-adherent culture inside microwells for up to >50 days after formation. Collection/harvest of microspheres was performed by gently pipetting the culture media in the AggreWell up and down bringing the microspheres into suspension and collecting the suspension in a tube pre-coated with anti-adherence solution (Stem Cell Technologies). For assessing microsphere composition, maturation, and fibre outgrowth capacity, microspheres were seeded onto poly-L-ornithine/laminin-521 coated plates and maintained in adherent culture for 48-72 hours.

Immunocytochemistry: Cells were fixed in 4% paraformaldehyde (Alfa Aesar) for 10 minutes. Unspecific antibody binding was blocked by incubating cells with PADT buffer; phosphate-buffered saline (PBS) without Ca²⁺ and Mg²⁺ (Gibco) with 0.02% sodium azide solution (Ampliqon), 0.5% Triton X-100 (Sigma), and 5% Donkey serum (Jackson Labs) for 30 minutes, followed by overnight incubation with primary antibodies at room temperature. The cells were washed 3 times with PBS without Ca²⁺ and Mg²⁺, blocked with PADT buffer for 10 minutes, and incubated with fluorophore-conjugated secondary antibodies for 2 hours at room temperature, protected from light. The cells were then counterstained with DAPI (10 pg/mL) for 5 minutes at room temperature, washed 3 times with PBS without Ca²⁺ and Mg²⁺, and stored at 4° C. in PBS without Ca²⁺ and Mg²⁺ supplemented with 0.02% sodium azide. Images were captured with an Olympus IX-81 or Olympus IX2-UCB microscope using CellSens software (Olympus). Primary antibodies: FOXA2 (1:100, SantaCruz), OTX2 (1:500,R&D), SOX2 (1:300, R&D), Ki-67 (1:250, Invitrogen), tyrosine hydroxylase (1:500, Pel-Freez), NEUN (1:500, Abcam), BRN2 (1:100, Santa Cruz), TBR1 (1:200, Abcam), PAX6 (1:500, Abcam), beta-III tubulin (1:1500, Promega and Abcam). Secondary antibodies: donkey anti-mouse IgG AF488/AF555 (1:800, Thermo Fisher), donkey anti-goat IgG AF488/AF555 (Thermo Fisher), donkey anti-rabbit IgG AF555/AF488/AF647 (1:800, Thermo Fisher).

Quantitative analysis of neural microsphere composition: Quantitative analysis of immunocytochemistry (ICC) was performed on randomly selected image fields from two replicate wells, three different fields of view for each well (n=3-6 experiments). For each field of view, images of the relevant channels (405 nm, 488 nm, 555 nm, 647 nm) were acquired at 10× magnification using a Olympus IX-81 or Olympus IX2-UCB microscope and CellSens software (Olympus). Quantitation of nuclear markers (SOX2, NEUN, Ki-67, FOXA2, OTX2) was done by manual counting in Photoshop (Adobe); cells showing a clear nuclear signal overlapping with DAPI were considered positive for the particular marker.

Microsphere diameter measurements: Images of microspheres inside microwells were acquired using a 4× and a 10× objective 48-72 hours after microsphere formation and at additional timepoints along the static maturation culture. The microsphere diameters were measured in 4X images using the horizontal measurement tool in CellSens software (Olympus). Violin plots were generated and mean and standard deviation (SD) calculated in GraphPad Prism version 8.

Cryopreservation of NSPBCs and neural microspheres: NSPBC adherent cultures were dissociated with accutase or EDTA (0.5 mM) into a single cell suspension, pelleted by centrifugation, resuspended in a cryoprotective solution (STEM-CELLBANKER®, Zenoaq), transferred to cryovials, placed in CoolCell® containers (Corning) in a −80° C. freezer overnight, and the next day transferred to vapour phase liquid nitrogen storage. For cryopreservation of microspheres, the microspheres were collected from the microwells by gently pipetting the culture media up and down bringing the microspheres into suspension and collecting the suspension in a tube pre-coated with an anti-adherence solution (Stem Cell Technologies). The microspheres were then gently forced to the bottom of the tube by centrifugation at 40-100 g for 1 minute and resuspended in either a cryoprotective solution comprised of neurobasal media supplemented with B27 (10%), N2 (2%), BDNF (80 ng/mL), GDNF (80 ng/mL) and DMSO (10%) or a commercially available cryoprotectant to a concentration of approximately 4800 microspheres/mL (100 cells/microsphere) or 1200 microspheres/mL (500 cells/microsphere). The microsphere suspension was then transferred to cryovials (0.5 mL/vial), placed in CoolCell® containers (Corning) in a −80° C. freezer overnight, and the next day transferred to vapour phase liquid nitrogen storage.

Glass capillary pulling for transplantation of NSPCs and neural microspheres: PC-100 puller (Narishige group) was used for pulling of capillaries. For transplantation of microspheres, capillaries of wider inner diameters than typically used for NSPCs were generated by slow pulling of the glass at 60° C. The inner diameter of these capillaries were <250 μm.

Intra-cerebral transplantation of NSPCs and neural microspheres to adult nude rats: All animals were conducted under European Union directive constrictions. The rats received a unilateral injection into the striatum by stereotaxic surgery of either hPSC-derived ventral midbrain NSPCs (DIV16) or neural microspheres generated from the same batches of midbrain NSPCs and matured in static non-adherent culture for an additional 14-16 days (DIV30-32). A total of 4 rats were transplanted with 100,000 ventral midbrain NSPCs and taken down 4 weeks post-transplantation. A total of 13 rats received a unilateral dose of microspheres (100 cells/microsphere) corresponding to 100,000 cells delivered in two deposits of 1 μL/deposit using coordinates described by Kirkeby and colleagues (Kirkeby et al, Cell Stem Cell, 2017). Rats transplanted with microspheres were sacrificed at 4 weeks post-transplantation (n=6) or 8 weeks post-transplantation (n=7). For transplantation, microspheres were collected 0.5-mL tubes pre-treated with anti-adherence solution, pelleted by centrifugation at 200 g for 1 minute, resuspended in Hank's buffered saline solution (HBSS) without Ca²⁺ and Mg²⁺ for a concentration of approximately 500 microspheres/pL (i.e. approximately 50,000 cells/pL) and placed on ice. Prior to any cell preparation or transplantation procedure, glass capillaries, pipette tips and other plasticware were pro-coated with anti-adherence solution.

Immunohistochemistry and histology: The rats were perfusion fixed, and the brains were collected, post-fixed in 4% PFA for 24 hours, and cryopreserved in a 30% sucrose solution. The brains were then sectioned coronally on a freezing sledge microtome at a thickness of 35 μm in series of 1:10 or 1:12. Immunohistochemistry was performed on free-floating sections and all washing steps were done with PBS without Ca²⁺ and Mg²⁺ with 0.02% sodium azide. The sections were washed three times and then incubated in PADT buffer for 30 min at room temperature in order to block unspecific antibody binding. The sections were then incubated with primary antibodies diluted in PADT overnight at room temperature. The sections were washed twice with PBS without Ca²⁺ and Mg²⁺ with 0.02% sodium azide, incubated with PADT blocking solution for 30 minutes, and then incubated with fluorophore-conjugated secondary antibodies for 2 hours at room temperature, protected from light. Lastly, the sections were counter-stained with DAPI (10 pg/mL) for 15 minutes at room temperature, washed 3 times with PBS without Ca²⁺ and Mg²⁺. Stained sections were then mounted on gelatine coated glass slides, cover slipped with PVA-DABCO, sealed with clear nail polish, and stored at 4° C. protected from light. Primary antibodies: FOXA2 (1:100, SantaCruz), OTX2 (1:400, R&D), SOX2 (1:200, R&D), Ki-67 (1:200, Invitrogen), tyrosine hydroxylase (1:500, Pel-Freez), NEUN (1:400, Abcam), NCAM (1:500, Santa Cruz) and human nuclear antigen (HNA) (1:100, Abcam). Secondary antibodies: donkey anti-mouse IgG AF488/AF555 (1:600, Thermo Fisher), donkey anti-goat IgG AF488/AF555 (1:600, Thermo Fisher), donkey anti-rabbit IgG AF555/AF488/AF647(1:600, Thermo Fisher).

Flow Cytometry and statistical analysis: Cryopreserved NSPCs were thawed and resuspended in HBSS-/-with 0.5% human serum albumin (HSA), counted on a NucleoCounter NC-200, stained with a fixable violet viability dye (1pL per 10⁶ cells, Invitrogen) for 15 min at room temperature protected from light, and then fixed and permeabilised using the BD Transcription Factor Buffer Set (BD Biosciences) according to the manufacturer's instructions. The fixed cells were then stained with fluorescently conjugated antibodies, and the samples acquired on a BD LSR Fortessa or BD FACSymphony (BD Biosciences). The fcs files were exported and analysed in FlowJo 10.5.03. Gates were set based on unstained control, fluorescence minus one (FMO) control, or biological negative control samples. Antibodies: FOXA2 (1:320, Miltenyi), OTX2 (1:320, Miltenyi), SOX2 (1:40, BD), SOX1 (1:320, Miltenyi), NKX6.1 (1:640, BD), OCT3/4 (1:10, BD), Nanog (1:1920, Biolegend).

Transcriptomic assays: For single cell RNA sequencing, NSPBC cultures were dissociated into single cell suspensions with accutase, and 3000-10000 cells were processed using the 10× Genomics Chromium Platform, and sequenced on a NextSeq550. For bulk RNA assessments, RNA was extracted from snap-frozen cell pellets ana analysed by NanoString.

Statistical analysis: Statistical analysis was performed using Prism 8.0.2 (GraphPad Software Inc., San Diego, Calif, USA). All graphs except violin plots are presented as mean ±standard deviation (SD).

### REFERENCES

Kirkeby A, Nolbrant S, Tiklova K, Heuer A, Kee N, Cardoso T, Ottosson D R, Lelos M J, Rifes P, Dunnett S B, Grealish S, Perlmann T, Parmar M. Predictive Markers Guide Differentiation to Improve Graft Outcome in Clinical Translation of hESC-Based Therapy for Parkinson's Disease. Cell Stem Cell. 2017 Jan 5;20(1):135-148. doi: 10.1016/j.stem.2016.09.004. Epub 2016 Oct. 27.

### Example 2

### Differentiation of hPSCs to and characterization of forebrain, midbrain and spinal cord NSPBCs for use as input cells for neural microsphere generation

The developing embryo is divided into 3 major germ layer lineages, the ectoderm, mesoderm and endoderm. The central nervous system (CNS) is formed within the ectoderm and in this process cells acquire a neural identity. Neural differentiation, as with all lineages, proceeds in a sequential manner with cells acquiring first an immature phenotype that typically is characterised by proliferation, mitosis, self-renewal and multipotency, and over developmental time cells typically lose these properties becoming post-mitotic, unable to self-renew and not potent and at this point are considered terminally differentiated. This process is well observed in the neural lineage, where at the earliest developmental stage cells are classified as highly multipotent neural stem/precursor cells (NSPCs) that can self-renew and give rise to numerous sub-lineages of cells (i.e. many classes of neurons, different types of astrocytes and oligodendrocytes) as well as themselves. In development and differentiation in vitro NSPCs diminish and are replaced by progeny intermediate cell types typically referred to as neuroblast cells (NBCs) or radial glial cells or intermediate precursor cells that typically are only unipotent and can self-renew only to a limited extent. NBCs finally give rise to terminally differentiated cell types such as neurons or astrocytes, which cannot further differentiate and in the case of neurons can no longer proliferate or self-renew at all.

Differentiation to these stages of neural cells has been achieved in vitro, replicating observed mammalian neurodevelopment, and has been observed or all 3 major regions of the CNS. hPSCs can be differentiated to all three major regions of the CNS, the forebrain, midbrain and hindbrain/spinal cord, and in these protocols the stages of hPSC to NSPCs to NBC to neuron has been extensively documented. For example, high purity protocols have been described to specify dorsal forebrain cells of the cortical glutamatergic neuronal lineage (Shi et al 2011; Shi et al 2012), ventral midbrain cells of the dopaminergic neuronal lineage (Kirkeby et al 2012, Nolbrant et al Parmar 2017) and ventral spinal cord cells of the motoneuron lineages (Amaroso et al 2013; Du et al 2015).

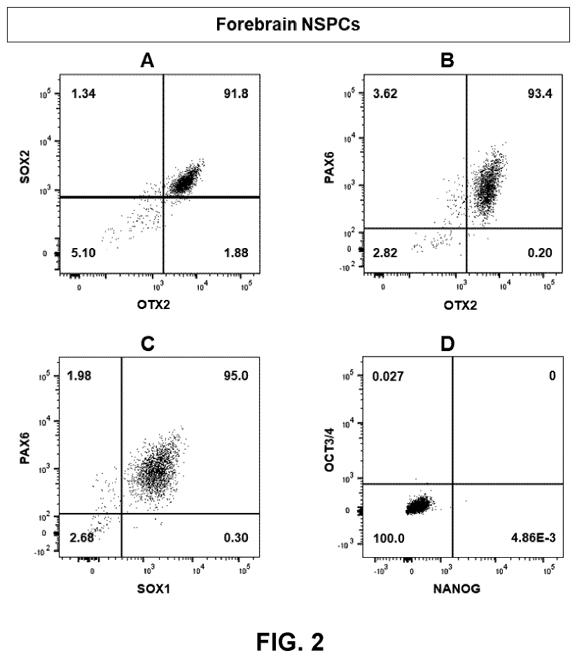

FIGS. 1-4 and 13 shows in vitro characterization hPSC-derived dorsal forebrain cortical NSPBCs used for the formation of forebrain microspheres. Differentiation to cortical NSPBCs is shown by immunocytochemical expression of broad NSPC markers SOX2 (FIG. 1D-E) and cortical specific NSPC markers PAX6 and OTX2 (FIG. 1A-C). Cells were further confirmed to be of a dorsal forebrain cortical lineage by flow cytometry that showed cells expressed cortical NSPC markers SOX2, OTX2, PAX6 and SOX1 (FIG. 2, 3) where cells were >90% SOX2+/OTX2+ (FIG. 2A, 3), >90% PAX6+/OTX2+ (FIG. 2B, 3), >90% PAX6+/SOX1+ (FIG. 2C, 3) and as expected did not express markers of pluripotency such as OCT3/4 and NANOG (FIG. 2D, 3). Cortical NSPCs have a typical rosette morphology where cells arrange in a circular orientation (FIG. 4A) and this, in addition to the aforementioned markers of SOX2/PAX6/OTX2, are indicative of a proliferative state and phenotype of cells that can be disassociated to a single cell suspension for passaging or transplantation to the CNS (Shi et al 2011; Espuny-Camacho et al 2013; Espuny-Camacho et al 2018). Measures of mRNA transcripts are further indicative of the acquisition of a regional CNS identity in the NSPCs produced from this differentiation; specifically, the upregulation of transcripts for PAX6 and OTX2 concomitant with an absence of EN1, FOXA2, LMX1A, NKX6.1 is indicative of a dorsal forebrain CNS regional commitment (FIG. 13). Forebrain NSPCs can be differentiated further to NBCs in this protocol and lineage with the gene TBR2 indicative of a dorsal forebrain cortical intermediate precursor/radial glial cell type, which is expressed at highest levels from DIV25 and beyond (FIG. 1B of reference Shi et al 2011), and further matured into neurons (FIG. 4B).

FIGS. 5-10 and 13 demonstrate differentiation in vitro of hPSCs to ventral midbrain NSPBCs used for the formation of midbrain microspheres. Differentiation to ventral midbrain NSPBCs was shown by expression of broad NSPC marker SOX2at 96.5% (FIG. 6A) and ventral midbrain specific NSPC markers FOXA2 and OTX2 were 96.7% and 93.6%, respectively, with a simultaneous absence of the non-ventral midbrain marker PAX6 (FIG. 6 B, C). Ventral midbrain NSPCs are indicative of a proliferative state based on this constellation of markers FOXA2/OTX2/SOX2 (FIGS. 5-6) and clearly exemplified by single cell RNA sequencing (scRNA-seq) of these DIV16 cells that shows they are heavily comprised of other NSPC markers such as NES, DCX and the proliferation marker MK167 (FIG. 8A). These NSPC markers of a ventral midbrain regional identity and proliferative state define a cell type that can be disassociated to a single cell suspension for passaging or transplantation to the CNS which has been extensively reported (Kriks et al 2011; Kirkeby et al 2012; Niclis et al., 2016; Gantner et al 2020). Midbrain NSPBCs should not express markers of pluripotency, and analysis at the protein level with flow cytometry showed these midbrain NSPCs at DIV16 did not express key markers OCT3/4 or NANOG and co-expression was importantly not detected (FIG. 7B), in contrast with positive control hPSCs (FIG. 7A). This was confirmed at the transcriptional level by scRNA-seq analysis showing that the ventral midbrain NSPCs did not co-express the major pluripotency makers POU5F1 (aka OCT3/4), NANOG, CD9 and PODXL (FIG. 8B). Measures of mRNA transcripts are further indicative of the acquisition of a regional ventral midbrain CNS identity in the DIV16 NSPCs produced from this differentiation; specifically the upregulation of transcripts for FOXA2, OTX2 and LMX1A concomitant with an absence of PAX6 (FIG. 13). At DIV16 of the differentiation, ventral midbrain cultures were comprised of NSPCs and did not contain NBCs as seen by a the near complete expression of the NSPC marker SOX2 and absence of the ventral midbrain NBC marker ASCL1(FIG. 9A; Arenas et al 2015). Importantly, these NSPCs can be further differentiated to a NBC stage such that by DIV26>30% of the cells were seen to express the NBC marker ASCL1(FIG. 9B).

FIGS. 11-13 demonstrate differentiation in vitro of hPSCs to towards ventral hindbrain/spinal cord NSPBCs used for the formation of hindbrain/spinal cord microspheres.

Differentiation to ventral hindbrain/spinal cord NSPBCs is shown by expression of broad NSPC marker SOX2 in >80% of the cells (FIG. 12A) and the ventral spinal cord NSPC marker NKX6.1 in >72.5% of the cells (FIGS. 11A-B, 12B). For NSPBCs to be of a caudal neural tube identity and thus of the hindbrain/spinal cord lineage they must concomitantly have an absence of the forebrain-midbrain NSPC marker OTX2 as was shown by immunocytochemistry and flow cytometry (FIGS. 11C-D, 12A-B). Ventral hindbrain/spinal cord NSPCs are characterized by a proliferative state and can be disassociated to a single cell suspension for passaging or transplantation to the CNS (Amoroso et al., 2013; Du et al., 2015). Measures of mRNA transcripts were further indicative of the acquisition of a regional CNS identity in the NSPCs produced from this differentiation; specifically the upregulation of transcripts for FOXA2 and NKX6.1 concomitant with an absence of PAX6, LMX1A, OTX2 and EN1 is indicative of a ventral hindbrain/spinal cord CNS regional commitment (FIG. 13).

### REFERENCES

2011. Nature Neuroscience. Shi et al., Livesey. Human cerebral cortex development from pluripotent stem cells to functional excitatory synapses
2011. Nature. Kriks et al Studer. Dopamine neurons derived from human ES cells efficiently engraft in animal models of Parkinson's disease
2012. Nature Protocols. Shi et al Livesey. Directed differentiation of human pluripotent stem cells to cerebral cortex neurons and neural networks
2012. Cell Reports. Kirkeby et al Parmar. Generation of Regionally Specified Neural Progenitors and Functional Neurons from Human Embryonic Stem Cells under Defined Conditions
2013. Cell Reports. Espuny-Camacho et al Vanderhaeghen. Pyramidal Neurons Derived from Human Pluripotent Stem Cells Integrate Efficiently into Mouse Brain Circuits In Vivo
2013. Journal of Neuroscience. Mackenzie W. Amoroso et al., Hynek Wichterle. Accelerated High-Yield Generation of Limb-Innervating Motor Neurons from Human Stem Cells
2015. Nature Communications. Du et al., Zhang. Generation and expansion of highly pure motor neuron progenitors from human pluripotent stem cells.
2016. Stem Cell Translational Medicine. Niclis et al Parish. Efficiently Specified Ventral

Midbrain Dopamine Neurons From Human Pluripotent Stem Cells Under Xeno-Free Conditions Restore Motor Deficits in Parkinsonian Rodents
2017. Nature Protocols. Nolbrant et al., Kirkeby. Generation of high-purity human ventral midbrain dopaminergic progenitors for in vitro maturation and intracerebral transplantation.
2015. Development. Arenas, Denham Villaescusa. How to make a dopamine neuron.
2018. Cell Reports. Espuny-Camacho et al Vanderhaeghen. Human Pluripotent Stem-Cell-Derived Cortical Neurons Integrate Functionally into the Lesioned Adult Murine Visual Cortex in an Area-Specific Way
2020. Cell Stem Cell. Gantner et al Parish. Viral Delivery of GDNF Promotes Functional Integration of Human Stem Cell Grafts in Parkinson's Disease

### Example 3

### Differentiation of NSPBCs to Neurons in Standard 2D Procedures Produces Fragile Cultures that cannot be Disassociated or Transported

The typical way to transfer NSPBCs from one condition to another, is to disassociate adherent or suspension NSPBCs to a single cell suspension. To do this, in vitro NSPBCs as described in Example 2 are treated with chelators or enzymatic disassociation agents that disrupt adhesion/binding proteins or disrupt molecular bonds between cells and/or the surfaces they are adhered onto, causing them to disassociate into single cell suspensions or clusters of a small number of cells. Example disassociation agents include Accutase, Trypsin, EDTA, Collagenase, Dispase and others. This process is routinely performed on NSPBCs for continued differentiation to terminal fates or transplantation to the CNS, and has been reported for all CNS lineages including dorsal forebrain cells (Shi et al., 2011; Espuny-Camacho et al., 2018), ventral midbrain (Kriks et al 2011; Kirkeby et al 2012; Niclis et al., 2016; Gantner et al 2020) and ventral spinal cord (Amaroso et al., 2013; Du et al., 2015). This final passaging and differentiation to terminal cell types has been described for neurons from forebrain, midbrain and spinal cord by protocols aforementioned and has been shown by the inventors to show NSPBCs used for generating microspheres have the capacity to generate neurons in standard conditions. Specifically, forebrain NSPBCs can be further differentiated in 2D adherent culture to neurons of a glutamatergic neuron identity, developing extensive neurite fibres typical of all neurons that are fragile and inextricably entangled and which cannot be enzymatically disassociated (FIG. 4B) and expressing makers of cortical glutamatergic neurons such as TBR1 and BRN2 (Shi et al., 2011). Furthermore, ventral midbrain NSPBCs can be further differentiated to a terminal neuronal fate as per standard 2D culture methodologies previously described (Kriks et al., 2011; Nolbrant et al., 2017) where cells develop extensive neurite fibres expressing pan-neuronal markers such as BETA-Ill-TUBULIN that are fragile and inextricably entangled (FIG. 10C,D), some of which are ventral midbrain dopaminergic neurons as shown by staining for tyrosine hydroxylase (TH) (FIG. 10E,F). Furthermore, ventral spinal cord NSPBCs can be further differentiated to neurons of a ventral spinal motoneuron identity, developing extensive neurite fibres typical of all neurons that are fragile and inextricably entangled and which cannot be enzymatically disassociated (FIG. 11E) and expressing markers of ventral motoneurons such as HB9 and ISL1 (Amaroso et al., 2013; Du et al., 2015).

Neurons derived from these protocols and throughout the entire field are conversely not disassociated; this is due to the architectural fragility of neurites that extend out from the cell body of neurons that are absent in NSPBCs; FIG. 4 (forebrain) and FIG. 10 (midbrain) and

FIG. 11 (Spinal cord) demonstrate these stark contrasts at a morphological and immunocytochemical level. There are no literature reports or inventions that describe methods for postmitotic neurons to be packaged into “compact and transportable” formats with no added compounds or specialised equipment that preserves their viability when harvested from the conditions they grow within (whether 2D adherent monolayers or 3D neurospheres or 3D organoids). Attempts by the inventors to disassociate neurons themselves to generate a compact and transportable format observed marked fragility and loss of this cell type. Terminally differentiated ventral midbrain cultures enriched for neuron and other terminal cell types (i.e. astrocytes and meningeal cells) are obtained at approximately DIV35-40 of the published protocol in the described 2D adherent culture conditions (FIG. 10C-F, Nolbrant et al., 2017). These cultures were incubated with the disassociating enzyme accutase at 37° C. in an attempt to generate a single cell suspension or small clusters of few cells. Neuron-rich cultures treated with accutase for a standard duration (25 minutes) yielded a mixture of single cells and small clusters that were shown to be deleterious for neurons (FIG. 46A, A′). This is show by re-plating where surviving cultures were depleted of most neurites and thus neurons (indicative of their death and loss during disassociation) and enriched for non-neuronal cell types without neurites (FIG. 46A, A′). Any observed neurites appeared to emanate from NBCs or young recently born neurons due to their short length that do not radiate far from their soma (FIG. 46A, A′). Incubation for 25 minutes was not sufficient to break up numerous thick and multi-layered areas of these extended adherent 2D cultures, and a longer accutase incubation procedure (90 minutes) was required to process the neuronal cell cultures in their entirety into a single cell suspension or small clusters, as evidenced by the increased number of cells growing following re-plating (FIG. 46B). Incubation for 90 minutes was, however, shown to also be deleterious for neurons. This is show by re-plating where surviving cultures were depleted of most neurites and thus neurons (indicative of their death and loss during disassociation) and enriched for non-neuronal cell types without neurites (FIG. 46B,B′). Any observed neurites appeared to emanate from NBCs or young recently born neurons due to their short length that do not radiate far from their soma (FIG. 46B,B′). These observations are further evident by immunostaining for the post-mitotic neuronal nuclei marker NEUN in DIV35 disassociated cultures, which were almost entirely devoid of NEUN+ nuclei upon re-plating whether subjected to accutase incubation for short (25 minutes, FIG. 47A, B) or long periods (90 minutes, FIG. 47C, D). This in stark contrast to neural microspheres (described in Examples 4-6) which can be differentiated to terminal neuronal cell fate by static non-adherent culturing without matrices (FIG. 46C,C′). Microspheres can be re-plated back to 2D adherent conditions without the need for disassociation and were seen to produce prodigious numbers of neurites that rapidly colonise surfaces and show no signs of proliferative NSPBCs (FIG. 46C,C′) and are enriched for the neuronal marker NEUN (FIG. 47E-F).

### REFERENCES

2011. Nature. Kriks et al Studer. Dopamine neurons derived from human ES cells efficiently engraft in animal models of Parkinson's disease
2011. Nature Neuroscience. Shi et al., Livesey. Human cerebral cortex development from pluripotent stem cells to functional excitatory synapses
2012. Nature Protocols. Shi et al Livesey. Directed differentiation of human pluripotent stem cells to cerebral cortex neurons and neural networks
2012. Cell Reports. Kirkeby et al Parmar. Generation of Regionally Specified Neural Progenitors and Functional Neurons from Human Embryonic Stem Cells under Defined Conditions
2013. Cell Reports. Espuny-Camacho et al Vanderhaeghen. Pyramidal Neurons Derived from Human Pluripotent Stem Cells Integrate Efficiently into Mouse Brain Circuits In Vivo
2013. Journal of Neuroscience. Mackenzie W. Amoroso et al., Hynek Wichterle. Accelerated High-Yield Generation of Limb-Innervating Motor Neurons from Human Stem Cells
2015. Nature Communications. Du et al., Zhang. Generation and expansion of highly pure motor neuron progenitors from human pluripotent stem cells.
2016. Stem Cell Translational Medicine. Niclis et al Parish. Efficiently Specified Ventral Midbrain Dopamine Neurons From Human Pluripotent Stem Cells Under Xeno-Free Conditions Restore Motor Deficits in Parkinsonian Rodents
2017. Nature Protocols. Nolbrant et al., Kirkeby. Generation of high-purity human ventral midbrain dopaminergic progenitors for in vitro maturation and intracerebral transplantation.
2018. Cell Reports. Espuny-Camacho et al Vanderhaeghen. Human Pluripotent Stem-Cell-Derived Cortical Neurons Integrate Functionally into the Lesioned Adult Murine Visual Cortex in an Area-Specific Way

### Example 4

### Formation of Neural Microspheres with Reproducible Morphology and Size

An invention which delivers the ability to physically move neurons in small compact formats and preserves their viability would provide significant advantages for biomedical research. The advantages of such an invention are best seen in the context of transplantation to the CNS for cell replacement therapies where neuron transplantation as opposed to NSPBC transplantation could provide several advantages. For example, neuron transplantation would deliver a post-mitotic cell population to a patient which ipso facto carries a low risk of tumour formation and cancer-like growth properties compared to mitotic and often highly proliferative phenotype of NSPBCs. Transplanting neurons is also theorised to facilitate a faster in vivo recovery where neurocircuit restoration is the goal as transplanted neurons already possess neurites and simply must innervate host targets for functional restoration, whereas NSPBCs must further differentiate to neurons and develop neurites before they can begin to innervate host targets. Furthermore, the transplantation of neurons allows the full differentiation protocol from hPSCs to the terminal cell type (i.e. neurons) to be conducted in vitro which allows for full process control of the differentiation procedure by human operators. Transplantation of

NSPBCs by contrast means that the last stages of the differentiation procedure to terminal cell fates occurs in vivo and is not controlled by human operators; this is believed to underlie the poor and variable in vivo purity of transplant derived neurons compared to in vitro derived neurons (Kriks et al, 2011; de Luzy et al, 2019; Kirkeby et al, 2017). The invention described herewithin details the method to package multipotent or unipotent NSPBCs into a format, termed microspheres, which allows for their differentiation to their terminal cell fate (i.e. neuron) but which also has the property of allowing these neurons to be in a compact and transportable format for movement to new conditions, for example into 2D adherent culture conditions (FIGS. 26-27, 31-33), passed through transplantation devices for neurosurgery (FIGS. 46C, C′, 47E-F) or transplanted to the CNS with such devices (FIGS. 47). The process for generating neural microspheres is as follows and summarised in a schematic (FIG. 14). Firstly, pre-differentiated NSPBCs derived from hPSCs were disassociated to single cells and quantified. Input NSPBCs used for microsphere formation spanned the major regions of the CNS as described in Example 2. Precise amounts of NSPBC were transferred to AggrewellTM400 Microwell 24-well plates (Stem Cell Technologies) containing 1200 microwells per well that were coated with an anti-adherence solution (Stem Cell Technologies) to prevent cell attachment and were prepared according to manufacturer's instructions. Centrifugal forces were applied to drive cells to the central point at the bottom of microwells and in close proximity to each other for the purpose of forming microspheres (FIGS. 15-22). Instead of centrifugal forces, passive environmental gravitational forces could be used instead as a simpler way to direct cells to the bottom of microwells for the purpose of aggregation which generated analogous neural microspheres (FIG. 18D).

Neural microspheres formed from NSPCs were seen to be consistent in morphology soon after formation, across CNS lineages of the forebrain, midbrain and hindbrain/spinal cord. Notably, all microwells were seen to comprise an individual microsphere in every instance that was also located at their centre and of a spherical morphology with minimal cell death (death being cells and material not within the microsphere and forming a cloud of debris) across CNS lineages used (FIGS. 15-22). Furthermore, a clear sharp border indicating tightly compacted and healthy neuroectoderm defining the outer circumference of the microsphere and was seen with forebrain microspheres (FIGS. 15-17), midbrain microspheres (FIG. 18-21) and hindbrain/spinal cord microspheres (FIG. 22). The same properties of individual microsphere formation per well, minimal cell death and sharp borders was observed within groups across a range of differently sized microspheres that were formed by seeding microwells with various numbers of cells including 50 cells (FIGS. 18A), 100 cells (FIGS. 15A, 18B, 22) or 500 cells (FIG. 15B, 17B, 18C).

The consistent and reproducible physical format of NSPC neural microspheres was reflected by consistent sizes as measured by their diameter soon after formation and which was observed across the lineages of the CNS. Specifically, DIV27forebrain NSPCs generated microspheres with little variation and a narrow range of diameter of 52.19±3.95 μm (mean ±SD) (100 cells/microsphere) and 90.23±6.24 μm (500 cells/microsphere) at 48-72 hours post-seeding (FIG. 23). DIV16 midbrain NSPCs similarly generated microspheres with little morphological variation and a narrow range of diameter of 37.61±4.51 μm (50 cells/microsphere) and 48.67±4.11 μm (100 cells/microsphere) and 91.24±5.65 μm (500 cells/microsphere) at 48 hours post-seeding (FIG. 24), and DIV20 NSPCs of hindbrain/spinal cord identity generated microspheres with little variation and a narrow range of diameters of 71.81±2.47 μm (100 cells/microsphere) at 48 hours post-seeding (FIG. 25).

Further differentiated cell types than NSPCs, specifically NBCs, were shown to also be amenable to processing and the generation of neural microspheres. This is shown by using as input cells for microspheres generation a range of CNS lineage NBCs including DIV34 forebrain TBR2 enriched cortical NBCs and DIV26 midbrain ASLC1 enriched NBCs described in Example 2. These NBC microspheres were seen to also generate individual spheroid clusters of small and consistent dimensions with a clearly delineated circumference/border and consistent dimensions soon after formation (FIG. 17, 20) and after a period of static non-adherent culture (FIG. 21). Furthermore, little cell death/debris was observed following NBC neural microsphere formation indicative of their amenability for microsphere preparation and was shown for the forebrain lineage with NBCs at DIV34 and observed at DIV40, at 100 cell (FIG. 17A) and 500 cell size (FIG. 17B). This was also shown with midbrain lineage NBCs formed at DIV26 and observed at DIV30 with 100 cell size (FIG. 20A) and 500 cell size microspheres (FIG. 20B).

### REFERENCES

2011. Nature. Kriks et al Studer. Dopamine neurons derived from human ES cells efficiently engraft in animal models of Parkinson's disease
2019. Journal of Neuroscience. de Luzy et al Parish. Isolation of LMX1a Ventral Midbrain Progenitors Improves the Safety and Predictability of Human Pluripotent Stem Cell-Derived Neural Transplants in Parkinsonian Disease
Kirkeby A, Nolbrant S, Tiklova K, Heuer A, Kee N, Cardoso T, Ottosson D R, Lelos M J, Rifes P, Dunnett S B, Grealish S, Perlmann T, Parmar M. Predictive Markers Guide Differentiation to Improve Graft Outcome in Clinical Translation of hESC-Based Therapy for Parkinson's Disease. Cell Stem Cell. 2017 January 5; 20(1):135-148. doi: 10.1016/j.stem.2016.09.004. Epub 2016 Oct. 27.

### Example 5

### Static Non-Adherent Culture of Neural Microspheres Differentiate NSPBCs to a Terminal Neuronal Fate Facilitates the Physical Transfer of Neurons

Neural microspheres formed from NSPBCs as described in Examples 2 and 4 spanning the major regions of the CNS were not immediately removed but cultured in microwells in static non-adherent conditions for further differentiation to a terminal cell fate (mainly neurons), for a period of days to weeks. The interval of days in vitro (DIV) selected of further differentiation of cortical forebrain and ventral midbrain lineages differed, 53 days in vitro (from DIV27-80) for the forebrain and 24 days in vitro (from DIV16-40) for ventral midbrain lineage; this is to compensate for the different rates of maturation of these lineages described in their in vitro 2D differentiation protocols (Shi et al., 2011; Kirkeby et al., 2016) and confirmed by the inventors (FIGS. 4, 10). These differences reflect the differential developmental time frames of various neural lineages and the successful outcomes of Example 5 and Example 6 will demonstrate the versatility of the microsphere invention across distinctly different regions of the CNS that possess different rates and properties of neurogenesis.

Across the periods of non-adherent static culture to differentiate neural microspheres no appreciable morphological differences were observed; microspheres retained an individual spherical cluster of cells within each microwell of consistent dimensions that remained small with a clearly delineated circumference/border and minimal cell death, observed in forebrain cortical microspheres differentiated from DIV27 to DIV60 at both 100 cell size (FIG. 15A, 16A) and 500 cell size (FIG. 15B, 16B) and ventral midbrain microspheres differentiated from DIV16 to DIV40 at both 50 cell size (FIG. 18A, 19A), 100 cell size (FIG. 18B, 19B) and 500 cell size (FIG. 18C, 19C). Additionally, when NBCs were used for microsphere formation at DIV26 (FIG. 17) and were further differentiated to terminal fate with static non-adherent culture conditions at DIV36 with both 100 cell sizes (FIG. 21A) and 500 cell sizes (FIG. 21B) sizes and morphologies of microspheres remained consistent, thus indicating robustness of the microsphere invention for use with input NBCs as well as NSPCs.

No appreciable change in the morphology and size of the microspheres is indicative of a committed pre-differentiated cell type (i.e. NSPBC) differentiating further to acquire a terminal cell fate (i.e. neuron), a key aspect of this invention as opposed to other reported methodologies of 3D culture and differentiation which typically are initiated with undifferentiated hPSCs. Such reported procedures initiated with hPSCs proliferate dramatically and produce large spheres often termed neurospheres or organoids of many thousands or even tens of thousands of cells. These often require passaging due to their extensive growth and size, often comprise an unhealthy necrotic core due to difficulties in nutrient diffusion through these large structures and are of such a large size they cannot be transplanted intact through the narrow surgical devices used for cell replacement therapy unless they are disassociated unlike the present invention (Denham et al., 2012; Niclis et al., 2013; Ebert et al., 2013; Lancaster et al., 2013; Jo et al., 2016).

The observations of minimal morphological changes over the period of static non-adherent culture was confirmed by measurement of microsphere diameters over time. Specifically, forebrain NSPC microspheres increased minimally if at all from a diameter soon after formation at DIV30 of 52.19±3.95 μm (mean±SD) (100 cells/microsphere) and 90.23±6.24 μm (500 cells/microsphere) to only 53.49±3.26 μm (100 cells/microsphere) and 99.03±5.95 μm (500 cells/microsphere) at DIV60 (FIG. 23). Likewise, midbrain NSPC microspheres increased minimally from a diameter soon after formation at DIV18 of 37.61±4.51 μm (mean ±SD) (50 cells/microsphere) and 48.67±4.11 μm (100 cells/microsphere) and 91.24±5.65 μm (500 cells/microsphere) to only 41.79±3.53 μm (50 cells/microsphere) and 57.55±4.73 μm (100 cells/microsphere) and 111.62±6.18 μm (500 cells/microsphere) at DIV40 (FIG. 24). Negligible differences in the sizes of microspheres across the period of static non-adherent culture and maturation reflects the fact that input cells were pre-differentiated and committed to a specific neural regional identity and close to the point at which they will become terminally differentiated; this is a preferred setup of the invention and a considerable difference compared to prior described spherical differentiation protocols that initiate with highly proliferative and fully undifferentiated/pluripotent hPSCs. Negligible changes in microsphere sizes over periods of static non-adherent culture also removes a need for additional effort and complication of disassociation and passaging of spheres, which is employed in other protocols to offset growth that occurs (Ebert et al., 2013).

To better understand and confirm the inventors' hypothesis that aforementioned observations of consistent morphologies and sizes of microspheres while in static non-adherent culture conditions actually represented committed pre-differentiated lineage cells (i.e. NSPBC) acquiring a terminal cell fate (i.e neuron), microspheres were collected and transferred longitudinally to a 2D in vitro analogue of the in vivo environment neural cells exist within for assessment; specifically, microspheres were seeded onto cultureware coated with poly-L-ornithine and laminin-521. Microspheres were collected from microwells by simple manual pipetting with standard handheld pipettes, static non-adherent culture conditions meant there was no requirement for disassociation agents and microspheres had not attached to or innervated the microwell surface area.

Longitudinal seeding to 2D systems (and following culture in these conditions for 48-72 hours) of forebrain neural microspheres across the period of their static non-adherent culture period (DIV27-80) revealed morphologically that cells progressed from a NSPBC state to a terminally differentiated neuronal state, despite collection and transport highlighting the advantage of this invention, and which is evidenced by features such as a gradual loss of migration and increased neurite outgrowth from seeded microspheres (FIGS. 26, 27). Shortly after 100 cell forebrain NSPC microspheres were formed at DIV27 they were seeded at DIV30 and were seen to comprise mostly of NSPBCs as characterised by the migration of cells out of the microsphere leading to a loss of a microsphere structure (FIG. 26A). Furthermore, cells observed were reminiscent of NBCs with small truncated fibres, a unipolar structure and migration far from the point at which they were seeded (FIG. 26A′). Further differentiation of forebrain microspheres to DIV40 revealed cells had matured and the spherical singular microsphere structure was better retained, with long neurites radiating out from the centre of the microsphere (FIG. 26B) however at closer magnification several migratory NBC cells are observable and the microsphere does not have a clearly delineated circumference and structure (FIG. 26B′). Further differentiation of the forebrain microsphere to DIV60 revealed the cells within the microsphere were mostly terminally differentiated neurons as the spherical singular microsphere structure was better retained and with long neurites radiating out from the centre of the microsphere (FIG. 26C). Closer examination revealed few migratory NBC cells (FIG. 26C′). Further maturation of the forebrain microsphere to DIV80 revealed the cells within the microsphere were seemingly entirely terminally differentiated neurons as the spherical singular microsphere structure was completely retained and with long neurites radiating out from the centre of the microsphere (FIG. 26D). Closer examination revealed no migratory NBC cells emanating from the microsphere (FIG. 26D′). Forebrain NBC microspheres formed at DIV34 and seeded at DIV40 were observed to also retain the capacity for collection and movement without compromise to cellular integrity and a neuronal capacity as seen by the generation of obvious neurites that emanated out from microsphere cores with both the 100 cell sized (FIG. 27A,A′) and 500 cell sized (FIG. 27B,B′) microspheres.

Longitudinal seeding to 2D cultures (and following culture for 48-72 hours in these conditions) of midbrain neural microspheres across the period of their static non-adherent culture period (DIV16-40) revealed morphologically that cells progressed from a NSPBC state to a terminally differentiated neuronal state, despite collection and movement highlighting the advantage of this invention, and which is evidenced by features such as a gradual loss of migration and increase in neurite outgrowth from seeded microspheres of 50 cell size (FIG. 31), 100 cell size (FIGS. 32) and 500 cell size microspheres (FIG. 33). Shortly midbrain NSPC microspheres were formed at DIV16 they were seeded at DIV18 and were seen to comprise mostly of NSPBCs as characterised by the migration of cells out of the microsphere leading to a complete loss of a microsphere structure and entirely flat monolayer (FIG. 26A). Furthermore, cells observed were reminiscent of NSPBCs with large cell bodies and a flat morphology, and if present fibres were small and truncated and from rare unipolar cells, and migration of cells far from the point at which they were seeded (FIG. 31A, 32A, 33A). Further differentiation of midbrain microspheres to DIV25 and DIV30 revealed cells had matured and the spherical singular microsphere structure was somewhat retained, with neurites radiating out from the centre of the microsphere however migratory NBC cells are observable and the microsphere does not have a clearly delineated circumference and structure (FIG. 31B-C,32B-C,33B-C). Further maturation of the midbrain microsphere to DIV35 revealed the cells within the microsphere were comprised mostly of terminally differentiated neurons as the spherical singular microsphere structure was better retained and many long neurites radiating out from the centre of the microsphere were observed and few migratory NBC cells were Seen (FIG. 31D,32D, 33D). Further differentiation of the midbrain microsphere to DIV40 revealed the cells within the microsphere were nearly entirely terminally differentiated neurons as the spherical singular microsphere structure was strongly retained and long neurites radiated out from the centre of the microsphere (FIG. 31E, 32E, 33E). Closer examination revealed few if any migratory NBC cells emanating from the microsphere (FIG. 31E′, 32E′, 33E′).

It is relevant to note that in static non-adherent culture conditions microspheres did not show any evidence of attachment in microwells, for example the absence of neurite growth out from microspheres onto microwell surfaces and/or to each other, despite considerable periods in these culture conditions, seen with forebrain microspheres over time (FIG. 15-16) and midbrain microspheres over time (FIG. 18-19). This was not due to a lack of neurons within microspheres as shown by their ready production of neurites following physical transfer without disassociation agents to adherent 2D conditions aforementioned here in Example 5, and results from immunocytochemical assessment in the following Example 6.

### REFERENCES

2012. Frontiers in Cellular Neuroscience. Denham, M et al., Thompson, L. H. (2012). Neurons derived from human embryonic stem cells extend long-distance axonal projections through growth along host white matter tracts after intra-cerebral transplantation.
2013. Frontiers in Cellular Neuroscience. Niclis et al., Cram. Characterization of forebrain neurons derived from late-onset Huntington's disease human embryonic stem cell lines
2013. Stem Cell Research. Ebert et al., Svendsen. E Z spheres A stable and expandable culture system for the generation of pre-rosette multipotent stem cells from human ESCs and iPSCs.
2013. Nature. Lancaster et al., Knoblich. Cerebral organoids model human brain development and microcephaly
2016. Cell Stem Cell. Jo et al., Ng. Midbrain-like Organoids from Human Pluripotent Stem Cells Contain Functional Dopaminergic and Neuromelanin-Producing Neurons

### Example 6

### Longitudinal Characterization of Neural Microsphere Composition and Maturation

As neural microspheres made from NSPBCs were held in static non-adherent culture in microwell plates for a prolonged period (up to >50 days) in a media suitable for the maintenance and maturation of neuronal cells, the cells comprising the microsphere were found to differentiate into neurons. This process is associated with a downregulation of key pan-CNS NSPC markers such as SOX2 and Ki-67, of which the latter labels specifically proliferating cells. These markers were monitored over time by seeding microspheres onto poly-L-ornithine/laminin-521 coated plates at DIV18, 25, 30, 35, and 40, and performing immunocytochemistry analysis 48 hours later. Shortly after microsphere formation (DIV30), constituent cells in microspheres made from forebrain NSPBCs at DIV27 were seen to highly express the NSPC marker SOX2 (FIG. 28B, 29B), while this expression decreased over time in static non-adherent culture conditions for maturation of forebrain neurons and by DIV60 were barely detected (FIG. 28E, 29E). This tendency was observed independently of whether the microspheres were made from 100 or 500 NSPBCs. Furthermore, the forebrain microspheres were seen to express the markers TBR1 and BRN2 which are selectively expressed in dorsal forebrain glutamatergic neurons (FIG. 30).

Likewise, 48 hours after formation (DIV18), ventral midbrain microspheres were found to be comprised mainly of cells expressing the markers SOX2 (FIG. 34-37) and Ki-67 (FIG. 38, 39), independently of whether the microspheres were made from 50, 100, or 500 cells. As observed for the forebrain neural microspheres, the proportion of cells expressing these markers declined over time. Specifically, at DIV18 ventral midbrain microspheres expressed 69.31±16.43% (mean ±SD) (50 cells/microsphere) and 74.47±10.91% (100 cells/microsphere) SOX2 (FIGS. 37) and 41.32±16.6% (50 cells/microsphere) and 28.94±11.12% (100 cells/microsphere) Ki-67 (FIG. 39). As expected, these levels gradually declined over time such that at DIV40, 12.40±6.57% (50 cells/microsphere) and 12.73±5.03% (100 cells/microsphere) of the cells expressed SOX2 (FIGS. 37) and 4.19±8.00% (50 cells/microsphere) and 0.78±0.95% (100 cells/microsphere) of the cells expressed Ki-67 (FIG. 39). The ventral midbrain lineage specific NSPC marker OTX2 was also seen to decrease from being expressed by 69.02±10.47% (50 cells/microsphere) and 72.70±12.72% (100 cells/microsphere) of the cells at DIV18 to 4.38±3.83% (50 cells/microsphere) and 4.61±4.36% (100 cells/microsphere) at DIV40 (FIGS. 42, 43). This tendency was not observed for the ventral midbrain lineage specific marker FOXA2 which is expected to be consistently expressed by both NSPBCs and dopamine neurons (FIG. 40); FOXA2 was expressed by 73.22±1.00% (50 cells/microsphere) and 82.71±7.37% (100 cells/microsphere) at DIV18 and by 62.21±12.25% (50 cells/microsphere) and 63.89±3.54% (100 cells/microsphere)at DIV40 (FIG. 41). While the proportion of cells in neural microspheres expressing NSPC related markers were reduced over time, the proportion of cells expressing markers known to label all neurons (e.g. beta-III tubulin and NEUN) (FIGS. 34-36, 42, 44) ora specific subtype of neurons such as the ventral midbrain dopaminergic neurons (e.g. tyrosine hydroxylase) (FIG. 40) gradually increased. For example, 48 hours after formation (DIV18) midbrain microspheres were found to contain only 0.54±0.43% (50 cells/microsphere) and 0.21±0.29% (100 cells/microsphere) cells expressing the pan-neuronal marker NEUN, while at DIV40 this number had increased to 64.95±10.62% (50 cells/microsphere) and 54.76±9.09% (100 cells/microsphere), indicating that the cells constituting the microspheres were indeed undergoing maturation from NSPBCs to neurons.

### Example 7

### Cryopreservation of Neural Microspheres

An essential property of hPSC-derived cell therapy products is the ability to cryopreserve the cells for long-term storage and distribution to clinical sites. It is therefore highly desired that neural microspheres after partial or full maturation in static non-adherent culture into terminal neural cell types can be cryopreserved. The typical challenge when attempting to cryopreserve spheres/clusters is poor survival and disintegration upon thaw likely due to insufficient penetration of cryoprotective solution to the core of the sphere/cluster. This issue may be overcome by generating microspheres that are uniform in size and small enough that the cryoprotectant is able to penetrate the entire structure. In order to test this experimentally, neural microspheres of 100 cells/microsphere and 500 cells/microsphere were generated from midbrain NSPBCs at 16 days in vitro (DIV) after initiating the differentiation and matured inside microwells for an additional 19 days as described in Example 5. At DIV35 the microspheres were collected from the microwells by gently pipetting the culture media up and down bringing the microspheres into suspension and collecting the suspension in a tube pre-coated with an anti-adherence solution (Stem Cell Technologies) also used for pre-coating the microwells prior to microsphere formation in order to prevent the microspheres from sticking to the plastic. The microspheres were then gently forced to the bottom of the tube by centrifugation at 40 g for 1 minute and resuspended in either a cryoprotective solution comprised of neurobasal media supplemented with B27 (10%), N2 (2%), BDNF (80 ng/mL), GDNF (80 ng/mL) and DMSO (10%) or a commercially available cryoprotectant (STEM-CELLBANKER®, Zenoaq) to a concentration of approximately 4800 microspheres/mL (100 cells/microsphere) or 1200 microspheres/mL (500 cells/microsphere). The microsphere suspension was then transferred to cryovials (0.5 mL/vial), placed in CoolCell® containers (Corning) in a −80° C. freezer overnight, and the next day transferred to vapour phase liquid nitrogen storage. The cryopreserved microspheres were thawed into neural maturation media, gently forced to the bottom of the tube by centrifugation at 40 g for 1 minute, resuspended in neural maturation media, and seeded onto poly-L-ornithine (Sigma)/laminin-521 (Biolamina) coated 96-well plates and cultured for 48 hours. The cryopreserved microspheres were seen to maintain their size and spherical shape (FIG. 45 B, B′) as compared to microspheres seeded fresh from the same batch (FIG. 45A, A′). Likewise, both fresh and cryopreserved spheres extended neurites within 48 hours after seeding (FIG. 45). The observed effects were independent of the cryoprotectant used. Together, these results indicate that the neuronal cells of which the microspheres were comprised survived the cryopreservation process and retained the capacity to extend neurites, which is essential for their application as a neuronal cell therapy.

### Example 8

### Transplantation of Neural Microspheres

One application of the neural microspheres is cell replacement therapy via intra-cerebral transplantation. Proof of concept showing that hPSC-derived neural microspheres comprised predominantly of post-mitotic neurons can survive transplantation and form a graft within the host brain tissue was obtained in rodents. Firstly, in vitro experiments were performed to test the ability of the neural microspheres to pass through the thin glass capillaries used for transplantation without being harmed by shear forces and without causing the capillaries to clog. First, the glass capillaries and plastic tubes were pre-coated with an anti-adherence solution (Stem Cell Technologies) to prevent the microspheres from sticking to the glass or plastic surfaces. The capillaries and tubes were filled with the anti-adherence solution, emptied, and then washed with HBSS without Ca²⁺ and Mg²⁺ prior to use. Microspheres of 100 cells were generated from hPSC-derived midbrain NSPBCs at 16 days in vitro (DIV) after initiating the differentiation and matured inside microwells for an additional 19 days as described in Example 5. At DIV35 the microspheres were collected from the microwells by gently pipetting the culture media up and down bringing the microspheres into suspension and collecting the suspension in a pre-coated tube. The microspheres were gently forced to the bottom of the tube by centrifugation at 40-200 g for 1 min and resuspended to a concentration of 500 microspheres/pL (i.e. 50,000 cells/pL) in neuronal maturation media. The concentrated microsphere solution was then passed through a capillary mimicking a transplantation setting and afterwards seeded onto poly-L-ornithine (Sigma)/laminin-521 (Biolamina) coated 96-well plates as described in Example 5. In order to prove the true value and applicability of neural microspheres, 2D neuronal cultures of the same age generated by adherent maturation culture from the same batch of midbrain NSPBCs were put through the same procedure. The 2D neurons were first detached from the culture plate and dissociated into a single cell suspension by incubation with accutase, which is the typical procedure when transplanting NSPBCs grown in adherent culture. The 2D neurons were exposed to accutase for either 25 minutes, the maximum time usually needed for dissociating NSPBCs, or 90 minutes, the time needed to properly dissociate these fibre-dense neuronal cultures into a single suspensions, spun down at 400 g for 10 minutes and resuspended in neuronal maturation media at a concentration of 50,000 cells/pL. The neuronal single cell suspension was then passed through a capillary and seeded in 96-well plates alongside the microspheres. At 48 hours after seeding, the microspheres displayed substantial neurite outgrowth (FIG. 46 C, C′) and were found by immunocytochemistry analysis to express the neuron marker NEUN (FIG. 47 E, F), indicating great survival of neurons within microspheres and a little harm induced by shear forces. In comparison, the cells obtained from 2D neuronal culture showed markedly less fibre outgrowth (FIG. A, A′, B, B′) and only sparse NEUN expression (FIG. 47A-D), suggesting that only a small fraction of the neurons had survived the procedure, while most of the neurons had been lost either during the dissociating process or due to shear stress. As a result, the majority of the seeded cells, which displayed a noticeably different morphology from typical 2D neuronal cultures (FIGS. 4B, 10, 11E), were likely residual NSPBCs or potential glial progenitor cells. Following the in vitro experiments, neural microspheres of midbrain identity were transplanted unilaterally into the striatum of adult nude rats in order to generate initial proof of concept in vivo. Microspheres made from 100 midbrain NSPBCs cells were collected at DIV30 as described above, concentrated to a solution of approximately 500 microspheres/pL (i.e. approximately 50,000 cells/pL) in HBSS without Ca²⁺ and Mg²+, and placed on ice. The microspheres were delivered intra-cerebrally in two deposits of 2 pL/deposit using a Hamilton syringe fitted with a pulled glass capillaries identical to those used for in vitro testing. Prior to the surgery, the glass capillaries were pre-coated with an anti-adherence solution (Stem Cell Technologies) as described above to prevent the microspheres from adhering to the glass surface and thus maximize the yield. The transplanted rats were sacrificed and their brains analysed by immunocytochemistry at 4 and 8 weeks post-transplantation (FIG. 48); at both time points viable grafts were observed in the striatum. Moreover, microsphere grafts showed strong, specific signals when stained with a human-specific antibody against NCAM, indicating that they were comprised of hPSC-derived neurons (FIG. 48 B, B′, D, D′).

Example 9

### Microspheres Comprised of hPSC-derived Pancreatic Islet-Like Cells

Cell replacement using hPSC-derived pancreatic islet-like cell clusters containing insulin-producing beta-cells and other endocrine cell types represents a promising therapy for patients with diabetes. Among the current challenges in the process of generating such cells are reproducibility, scale, and the ability to cryopreserve the cells. Pancreatic islet-like cells are typically generated from hPSCs in a 3D culture system allowing large scale production of islet-like clusters; however, cryopreservation of the clusters, which can be relatively large and heterogenous in size not allowing cryoprotective solutions to properly penetrate the clusters and protect the cells at the core, is a challenge. This is currently solved by dissociating the clusters into single cells, which may then be cryopreserved and re-aggregated by spontaneous cluster formation upon thaw. This procedure, however, comes at the cost of a lower yield, while the alternative, transplantation of single cells instead of clusters typically results in poorer survival of the cells and thus a worse outcome in vivo. The current invention addresses these challenges; by forming the differentiated cells into uniform size-controlled microspheres, which are small enough to allow cryopreservation of intact clusters of cells, the yield and reproducibility are markedly improved. In order to experimentally test this, pancreatic islet-like cells were obtained by differentiation of hPSCs using a proprietary 3D suspension culture protocol (US2014234963, US2012135519, US2015247123, WO20207998, US2019085295, WO20043292, US2020199540, Funa et al, Cell Stem Cell, 2015). Pancreatic islet-like cell identity was confirmed by flow cytometry analysis at 29 days in vitro (DIV) after initiating the differentiationDIV29 showing predominantly putative beta-like cells co-expressing c-peptide and NKX6.1 (56.8%, FIG. 49A) and a smaller proportion of putative alpha-like cells co-expressing c-peptide and glucagon (4.69%, FIG. 49E). The pluripotency markers OCT3/4 and Nanog were no longer detected at this stage (FIG. 50). At this stage of differentiation, the hPSC-derived pancreatic islet-like cell clusters are typically dissociated into a single cell suspension, cryopreserved and then thawed and re-aggregated for downstream applications such as transplantation. Re-aggregation of either fresh or cryopreserved single cells was performed by spontaneous aggregation in suspension, which is the standard approach, or by spin-aggregation of single cells into microspheres consisting of either 500 or 1000 cells. For microsphere formation, precise amounts of hPSC-derived pancreatic islet-like cells were transferred to Aggrewell™400 24-well plates (Stem Cell Technologies) containing 1200 microwells per well that were coated with an anti-adherence solution (Stem Cell Technologies)to prevent cell attachment and were prepared according to manufacturer's instructions. Centrifugal forces were applied to drive cells to the central point at the bottom of microwells and in close proximity to each other for the purpose of forming microspheres. Cells were cultured in the microwells in a media appropriate for the maintenance and maturation of pancreatic islet-like cells, and only for seeding was Y27632 added at 10 μM. Following incubation for 48 hours, the cells were seen to aggregate into individual spheroid clusters of small and consistent dimensions with a clearly delineated circumference/border (FIGS. 51, 52). The microsphere diameters were measured using phase-contrast images of microspheres inside microwells and CellSens software 48 hours after microsphere formation (FIG. 51) and showed that the diameter was 80.10±4.51 μm (mean±SD) and 103.89±3.74 μm for microspheres made from 500 and 1000 cells, respectively (FIG. 55). These measurements were confirmed by Biorep analysis of microspheres collected from the microwells 48 hours after re-aggregation (FIGS. 53, 54, 55); the microspheres were found to be uniform in shape and size, and the majority (84.6%) of the microspheres made from 500 cells had a diameter in the range of 50-100 μm, while the majority (91.3%) of microspheres made from 1000 cells had a diameter in the range of 101-150 μm (FIG. 54). Flow cytometry analysis performed at the same time showed that re-aggregation by microsphere formation rather than the standard spontaneous re-aggregation in suspension culture had no substantial impact on the expression of the key lineage markers c-peptide, NKX6.1, and glucagon (FIG. 49B-D, F-H), indicating that the microspheres were indeed capable of maintaining the relative proportions of alpha- and beta-like cells. In addition, the microspheres proved capable of cryopreservation. Microspheres of 1000 hPSC-derived pancreatic islet-like cells were collected from microwells at DIV31 and were frozen down in a cryoprotective solution (STEM-CELLBANKER®, Zenoaq). Upon thaw, the microspheres were kept in suspension culture for 24 hours and remained intact as consistently-sized spheres (FIG. 56). One of the challenges of the re-aggregation process of hPSC-derived pancreatic islet-like cells is that it is typically associated with a substantial cell loss and low yield; however, by re-aggregating the cells into spin-aggregated microspheres, the yield was increased from 29.6% after spontaneous cluster formation in suspension culture to 63.9% and 72.2% after formation of microspheres consisting of 500 and 1000 cells, respectively (FIG. 57).

### REFERENCES

Funa N S, Schachter K A, Lerdrup M, Ekberg J, Hess K, Dietrich N, Honore C, Hansen K, Semb H. β-Catenin Regulates Primitive Streak Induction through Collaborative Interactions with SMAD2/SMAD3 and OCT4. Cell Stem Cell. 2015 Jun. 4; 16(6):639-52. doi: 10.1016/j.stem.2015.03.008. Epub 2015 Apr. 23.
US2014234963 EFFICIENT INDUCTION OF DEFINITIVE ENDODERM FROM PLURIPOTENT STEM CELLS
US2012135519 INDUCED DERIVATION OF SPECIFIC ENDODERM FROM HPS CELL-DERIVED DEFINITIVE ENDODERM
US2015247123 GENERATION OF PANCREATIC ENDODERM FROM PLURIPOTENT STEM CELLS USING SMALL MOLECULES
WO20207998 GENERATION OF PANCREATIC ENDODERM FROM STEM CELL DERIVED DEFINITIVE ENDODERM
US2019085295 GENERATION OF FUNCTIONAL BETA CELLS FROM HUMAN PLURIPOTENT STEM CELL-DERIVED ENDOCRINE PROGENITORS
WO20043292 GENERATION OF FUNCTIONAL BETA CELLS FROM HUMAN PLURIPOTENT STEM CELL-DERIVED ENDOCRINE PROGENITORS
US2020199540 ENRICHMENT OF NKX6.1 AND C-PEPTIDE CO-EXPRESSING CELLS DERIVED IN VITRO FROM STEM CELLS

### Example 10

### Methodologies for Mesodermal (Cardiomyocyte) Microsphere Generation

Human pluripotent stem cell-derived cardiomyocyte-like cells and other mesodermal derivatives represent a promising cell source for cell therapies, drug and toxicity testing as well as suitable models to study diseases and development. Current cell therapy approaches aiming at regenerating myocardial tissue by replacement therapy using hPSC-derived cardiomyocyte are hampered by poor delivery, retention and engraftment of the cells following transplantation to the heart (Hastings et al, Adv Drug Deliv Rev, 2015; Feyen et al, Adv Drug Deliv Rev, 2016). Application of cardiac microspheres might overcome these challenges due to improved retention potential of microspheres in the myocardium as indicated from research on primary cardiospheres and cardiac progenitor cells (Cho et al, Mol Ther, 2012; Trac et al, Circ Res, 2019). Here we disclose an alternative method for generation and maintenance of size-controlled cardiac microspheres from human pluripotent stem cell derived cardiomyocyte-like cells with minimized risk of sphere fusion, free from use of biomaterials and/or extracellular matrix components and the option of long-term maturation in vitro followed by sphere harvest and use independent of cell detachment and/or dissociation reagents.

To generate cardiomyocytes in vitro, the human embryonic stem cell line XF3053 was maintained under feeder-free conditions in StemMACSTM iPS-Brew XF (Miltenyi) on LN521 (BioLamina) according to respective manufacturer's instruction. Cells were passaged every 3-4 days using accutase (Stem Cell Technologies) and seeded in StemMACSTM iPS-Brew XF supplemented with 10 μM Y-27632 (Sigma) at 1.6-2.4×10⁴ cells/cm² on T flasks (Nunc). Cell lines were tested negative for mycoplasma contaminations and karyotypic abnormalities throughout the study. Cardiomyocytes were generated applying a modified 3D differentiation protocol (Kempf et al, Nat Protoc, 2015; Halloin et al, Stem Cell Reports, 2109). In brief, cells were inoculated in 6-well suspension plates (Greiner) or 125m1 shaker flasks (Corning) for aggregate formation at 0.16×10⁶ cells/mL in StemMACSTM iPS-Brew XF supplemented with 10 μM Y-27632 and maintained on an orbital shaker (Infors Celltron) at 70 rpm. After 48 hours, differentiation was induced (termed day 0, DIVO) using 4-6 μM CHIR99021 (Tocris) for 24 h followed by 2 μM Wnt-059 (Tocris) for 24h in RPM11640 medium (Life Technologies) supplemented with 2% B27 without insulin (Life Technologies) or RPM11640 medium supplemented with 0.2 mg/mL L-ascorbic acid 2-phosphate (Sigma) and Albix (Albumedix). Cells were kept in RPM11640 supplemented with 2% B27 and 0.2 mg/mL L-ascorbic acid 2-phosphate (Sigma) from day 5 (DIV5) onwards. Cells were dissociated on day 8 (DIV8) for 8 minutes using STEMdiff™ Cardiomyocytes Support medium (Stem Cell Technologies) or accutase for further characterization and reaggregation experiments. In some experiments dissociated cardiomyocytes were cryopreserved using a cryoprotective solution (STEM-CELLBANKER®, Zenoaq) and stored in liquid nitrogen for subsequent reaggregations.

For cardiomyocyte reaggregation, AggrewellTM400 Microwell 24-plates (Stem Cell Technologies) containing 1200 cavities per well were prepared according to manufacturer's instruction and seeded with cardiomyocytes obtained after 8 days of differentiation (DIV8) with the indicated number of cells per cavity in 2m1 RPM11640 medium (Gibco, cat. no. 21875-034) supplemented with 2% B27 (Life Technology, cat. no. 17504), 0.2 mg/mlAscorbic-2-phosphate (Sigma, cat.no. A8960) and 10 μM Y-27632 (Sigma, cat.no. Y27632-Y0503). Aggregates were maintained in the microwells until final harvest with medium being exchanged every 3-4 days.

Samples on day 0 (DIV0) and 8 (DIV8) were subjected to flowcytometric analysis for AF647-conjugated OCT3/4 (BD, cat.no. 560329; dilution 1:100) and PE-conjugated cardiac Troponin T (BD, cat.no. 564767; 1:200). In brief, single cells obtained by dissociation using accutase (Stem Cell Technologies) were fixed in 4% formaldehyde (VWR) for 30-45 min, permeabilized and stained for 30 min at room temperature using PBS supplemented with 0.2% Triton X-100 (Sigma) and 5% donkey serum (NovusBio). Cells were washed between each step in PBS supplemented with 1% BSA (Miltenyi) and centrifugated at 800 g for 3 minutes. Samples were analyzed on a LSRFortessa™ Flow Cytometer (BD) and processed using FlowJo Software (Version 10.7).

Cluster size analysis was conducted using 200 μlsample on an automatic islet cell counter (biorep) with each sample being measured at least twice as technical repeat. plEQ represents a measure of cell mass based on digital image analysis methods (Buchwald et al, Cell Transplant, 2016). IPN indicates the absolute sphere count of the indicated size in a 200 μl sample. The average aggregate diameter was calculated based on the diameter calculated from the surface area of the wells divided by the number of particles assuming circular aggregate shape.

### REFERENCES

Buchwald P, Bernal A, Echeverri F, Tamayo-Garcia A, Linetsky E, Ricordi C. Fully Automated Islet Cell Counter (ICC) for the Assessment of Islet Mass, Purity, and Size Distribution by Digital Image Analysis. Cell Transplant. 2016 October; 25(10):1747-1761.
Cho H J, Lee H J, Youn S W, Koh S J, Won J Y, Chung Y J, Cho H J, Yoon C H, Lee S W, Lee E J, Kwon Y W, Lee HY, Lee SH, Ho WK, Park YB, Kim HS. Secondary sphere formation enhances the functionality of cardiac progenitor cells. Mol Ther. 2012 September;20(9):1750-66.
Feyen DAM, Gaetani R, Doevendans P A, Sluijter J P G. Stem cell-based therapy: Improving myocardial cell delivery. Adv Drug Deliv Rev. 2016 Nov. 15; 106(Pt A):104-115.
Halloin C, Schwanke K, Lobel W, Franke A, Szepes M, Biswanath S, Wunderlich S, Merkert S, Weber N, Osten F, de la Roche J, Polten F, Christoph Wollert K, Kraft T, Fischer M, Martin U, Gruh I, Kempf H, Zweigerdt R. Continuous WNT Control Enables Advanced hPSC Cardiac Processing and Prognostic Surface Marker Identification in Chemically Defined Suspension Culture. Stem Cell Reports. 2019 Oct. 8; 13(4):775.
Hastings C L, Roche E T, Ruiz-Hernandez E, Schenke-Layland K, Walsh C J, Duffy G P. Drug and cell delivery for cardiac regeneration. Adv Drug Deliv Rev. 2015 April; 84:85-106.
Kempf H, Kropp C, Olmer R, Martin U, Zweigerdt R. Cardiac differentiation of human pluripotent stem cells in scalable suspension culture. Nat Protoc. 2015 September; 10(9):1345-61. Trac D, Maxwell J T, Brown M E, Xu C, Davis M E. Aggregation of Child Cardiac Progenitor Cells Into Spheres Activates Notch Signaling and Improves Treatment of Right Ventricular Heart Failure. Circ Res. 2019 Feb. 15; 124(4):526-538.

### Example 11

### Microspheres Comprised of Stem Cell-Derived Cardiomyocytes

Cardiomyocyte differentiation efficiency was confirmed by flow cytometry for cardiac Troponin T (cTNT) after 8 days of differentiation (DIV8) with purities generally above 90% cTNT+ as shown in FIG. 58. At this stage, cells were essentially free of residual undifferentiated cells as indicated by <0.1% OCT3/4+ analysed by flow cytometry on the same day (FIG. 59). The floating 3D aggregates displayed a relative broad size distribution with 60% of the aggregates having a diameter between 200 to 400 μm and 5.8% above 400 μm (FIG. 60).

Aggregates were dissociated as described in Example 10 and subjected to microsphere formation seeding 50, 150, 500, 1000 or 1500 cells per cavity (FIG. 61-1 and FIG.

61-2), or 25, 50, 100 or 500 cells per cavity (FIG. 62). Automated cluster analysis of the spheres showed a clear increase in cell mass per mL with increasing cell numbers seeded per cavity (FIG. 63A) as well as the expected increase in sphere diameter ranging from <80 μm for 100 cells and >160 μm for 1500 cells (FIG.63B). Notably, size distribution of individual conditions confirmed uniformly sized clusters with 100 cells resulting in 90.7% of clusters having a diameter below 100 μm, 250 cells in 97% of spheres ranging from 51 to 200 μm and 1000 cells in 75% of spheres ranging from 151 to 200 μm (FIG. 64 and FIG. 65). The reduction and uniformity in size compared to control aggregates (default DIV8 aggregates from 3D differentiation) was confirmed from the images obtained from the biorep analysis shown in FIG. 66.

In order to confirm the cardiomyocyte identity and purity of the formed microspheres, representative condition of spheres (100 and 500 cells/microsphere) were plated on Laminin 521 for 24h in RPMI medium supplemented with 2% B27 and subsequently subjected to immunofluorescent staining of the cardiomyocyte-specific markers NKX2.5 and Sarcomeric Actinin (FIG. 67).

Together, the data show size-controlled formation of cardiac microspheres at a wide range of cell numbers ranging from as little as 25 cells to 1500 cells seeded per microcavity and forming individual spheres that can be maintained long-term without microsphere fusion. Notably, each condition resulted in a very narrow distribution in sphere size and thereby allows defined and controlled resizing of spheres obtained from an aggregate-based 3D differentiation process. The microspheres can be maintained for an extended period of time without fusion for further maturation purposes and subsequent transplantation studies.

### Example 12

### Cryopreservation and Delivery of Cardiac Microspheres

A further limitation in cell-based therapies is the long-term storage and lack of appropriate holding steps in the manufacturing process. Cryopreservation of partially or fully matured cardiomyocyte microspheres would provide a suitable holding step of a final microsphere drug product. Here we disclose a method that allows cryopreservation of human stem cell-derived cardiac microspheres. For this, microspheres of 1000 hESC-derived cardiomyocyte-like cells were generated by seeding Aggrewell™ microwells from cryopreserved single cells of DIV8 cardiomyocytes in reaggregation medium consisting of RPMI1640 media supplemented with B27 (2%), L-ascorbic acid 2-phosphate (0.21 mg/mL) and 10 μM Y-27632 (10 μM). After 3 days of reaggregation the microspheres were resuspended by gently pipetting the microspheres out into the surrounding suspension in the Aggrewell. The microsphere suspension was transferred to a tube, spheres settled down quickly and medium removed. Microspheres were resuspended in a cryoprotective solution (STEM-CELLBANKER®, Zenoaq) to a concentration of approximately 1200 microspheres/mL and transferred to a cryovial. The vial was transferred to a CoolCell® container and placed at −80° C. for 24 hours and transferred in liquid nitrogen for long term storage.

Cryopreserved microspheres were thawed in reaggregation media supplemented with DNAse I (50 pg/mL). Microspheres were spun down at 250 g for 3 minutes and resuspended in reaggregation media supplemented with DNAse I (50 pg/mL). The microspheres were seeded in a 6-well suspension plate and maintained on an orbital shaker (Infors Celltron) at 70 rpm.

Thawed microspheres showed a similar cellular morphology and sphere shape 4 days after cryopreservation (FIG. 68). Notably, regular beatings were observed within 24 h after thawing.

The present invention enables maintenance of individual cardiac microspheres over an extended period of weeks and month with minimal risk of sphere fusion. This makes the microspheres particularly suitable for controlled cell injection applying narrow syringe needles (e.g. G27 or G30 needles with a typical inner diameter of 210 μm and 159 μm, respectively) without affecting the microsphere integrity compared to previous approaches where microspheres are maintained in free-floating suspension cultures (Correia et al, Biotechnol bioeng, 2018) and prone to fusion. Furthermore, the herein described method of continuous microsphere culture allows for continuous maturation of cardiomyocytes in a 3D environment similar to engineered heart tissues (Tiburcy et al, Circulation, 2017), with the advantage of being injectable. Thereby controlled cell delivery of size-controlled mature miniature cardiac tissues becomes feasible, without the requirement of disrupting cell-cell interactions prior to injection. Consequently, we confirmed the feasibility of cell extrusion via a G30 syringe needle on microspheres. Notably, well-controlled extrusions without any signs of clotting or change in resistance was conducted for cardiac microspheres formed from 50 and 100 pluripotent stem cell-derived cardiomyocyte-like cells (FIG. 69), confirming optimal properties for microsphere delivery for in vivo application.

### REFERENCES

Correia C, Koshkin A, Duarte P, Hu D, Carido M, Sebastiäo M J, Gomes-Alves P, Elliott D A, Domian I J, Teixeira A P, Alves P M, Serra M. 3D aggregate culture improves metabolic maturation of human pluripotent stem cell derived cardiomyocytes. Biotechnol Bioeng. 2018 Mar; 115(3):630-644.
Tiburcy M, Hudson J E, Balfanz P, Schlick S, Meyer T, Chang Liao M L, Levent E, Raad F, Zeidler S, Wingender E, Riegler J, Wang M, Gold J D, Kehat I, Wettwer E, Ravens U, Dierickx P, van Laake L W, Goumans M J, Khadjeh S, Toischer K, Hasenfuss G, Couture L A, Unger A, Linke W A, Araki T, Neel B, Keller G, Gepstein L, Wu J C, Zimmermann W H. Defined Engineered Human Myocardium With Advanced Maturation for Applications in Heart Failure Modeling and Repair. Circulation. 2017 May 9; 135(19):1832-1847.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for obtaining a stem cell-based microsphere, comprising the steps of:
differentiating pluripotent stem cells (PSCs) to obtain differentiated cells,
aggregating the differentiated cells to form a stem cell-based microsphere, and
allowing the differentiated cells of the stem cell-based microsphere to further mature.

2. The method according to claim 1, wherein the PSCs are differentiated for at least 2 days prior to aggregating the differentiated cells.

3. The method according to claim 1, wherein the PSCs are differentiated for a period of time whereby at least 50% of the differentiated cells are no longer pluripotent, prior to the step of aggregation.

4. The method according to claim 1, wherein the differentiated cells are aggregated by spin-aggregation.

5. The method according to claim 1, wherein the stem cell-based microsphere is matured in an environment having low cell attachment properties.

6. A method for obtaining a neural microsphere, comprising the steps of:
differentiating PSCs into neural stem precursor blast cells,
aggregating the neural stem precursor blast cells to form a neural microsphere, and
allowing the neural stem precursor blast cells of the neural microsphere to further mature.

7. The method according to claim 6, comprising the additional step of seeding the neural stem precursor blast cells in a well suitable for maintaining a neural microsphere in a static non-adherent culture, prior to the step of aggregating the neural stem precursor blast cells.

8. The method according to claim 7, wherein the well is a microwell with low cell attachment properties.

9. The method according to claim 6, wherein from about 5 to about 1000 neural stem precursor blast cells are aggregated.

10. The method according to claim 6, wherein the PSCs are differentiated for a period of time whereby at least 50% of the neural stem precursor blast cells are no longer pluripotent, prior to the step of aggregating the neural stem precursor blast cells.

11. A neural microsphere comprising stem cell-derived neural cells, wherein the diameter of the neural microsphere is less than about 250 μm, and wherein at least 90% of the volume of the neural microsphere comprises neural cells.

12. The neural microsphere according to claim 11, comprising from about 5 to about 1000 neural cells.

13. The neural microsphere according to claim 11, wherein the surface of the neural microsphere consists of neural cells.

14. The neural microsphere according to claim 11, wherein the neural microsphere is free of exogenous extracellular matrix and/or free of exogenous hydrogel.

15. (canceled)

16. The neural microsphere according to claim 12, wherein the surface of the neural microsphere consists of neural cells.

17. The neural microsphere according to claim 16, wherein the neural microsphere is free of exogenous extracellular matrix and/or free of exogenous hydrogel.

18. A method of treating a neurological condition, comprising administering a neural microsphere according to claim 17.

19. The method according to claim 2, wherein the PSCs are differentiated for a period of time whereby at least 50% of the differentiated cells are no longer pluripotent, prior to the step of aggregation.

20. The method according to claim 19, wherein the differentiated cells are aggregated by spin-aggregation.

21. The method according to claim 20, wherein the stem cell-based microsphere is matured in an environment having low cell attachment properties.

22. The method according to claim 7, wherein from about 5 to about 1000 neural stem precursor blast cells are aggregated.

23. The method according to claim 22, wherein the PSCs are differentiated for a period of time whereby at least 50% of the neural stem precursor blast cells are no longer pluripotent, prior to the step of aggregating the neural stem precursor blast cells.
